(19) United States
(12) Patent Application Publication
(10) Pub. No.: US 2025/0250354 A1
(43) Pub. Date: Aug. 7, 2025

(54) NEW CLASS OF MOLECULES FOR SELECTIVE CLEARANCE OF ANTIBODY

(71) Applicant: OSE IMMUNOTHERAPEUTICS, NANTES (FR)

(72) Inventors: NICOLAS POIRIER, TREILLIERES (FR); CAROLINE MARY, SAINTE-PAZANNE (FR); AMANDINE GEORGES, NANTES (FR); LYSSIA BELARIF, REZE (FR)

(21) Appl. No.: 18/856,160

(22) PCT Filed: Apr. 13, 2023
(86) PCT No.: PCT/EP2023/059613

§ 371 Date: Oct. 11, 2024

(30) Foreign Application Priority Data
Apr. 13, 2022 (EP) 22305542.7

(51) Int. Cl. C07K 16/28; A61K 38/00; A61P 37/06; C07K 14/735

(52) U.S. Cl. CPC C07K 16/2896; A61P 37/06; C07K 14/70535; A61K 38/00; C07K 2319/30

(57) ABSTRACT

The present invention relates to a new class of molecules for selective clearance of antibodies and the uses thereof for treating antibody-mediated diseases and disorders.

### FIELD OF THE INVENTION

The present invention relates to the field of the medicine, especially of the treatment of diseases and disorders associated with antibodies. More particularly, it relates to molecules for selective clearance of antibodies.

### BACKGROUND OF THE INVENTION

Immunoglobulin gamma (IgG) antibodies play a key role in the pathology of many disorders, such as autoimmune diseases, inflammatory diseases, and disorders in which the pathology is characterized by over-expression of IgG antibodies (e.g., hypergammaglobulinemia).

The half-life of IgG in the serum is prolonged relative to the serum half-life of other plasma proteins in part because of the binding of the Fc region of IgG to a Fc receptor called FcRn (neonatal Fc receptor). FcRn functions to protect IgG from degradation. FcRn binds to pinocytosed IgG and protects the IgG from transport to degradative lysosomes by recycling it back to the extracellular compartment. This recycling is facilitated by the pH dependent binding of IgG to FcRn, where the IgG/FcRn interaction is stronger at acidic endosomal pH than at extracellular physiological pH. When the serum concentration of IgG reaches a level that exceeds available FcRn molecules, unbound IgGs are not protected from degradative mechanisms and will consequently have a reduced serum half-life. Thus, inhibition of IgG binding to FcRn reduces the serum half-life of IgG by preventing IgG endosomal recycling of IgG.

Based on this knowledge, agents that antagonize the binding of IgG to FcRn have been identified as useful for regulating, treating or preventing antibody-mediated disorders, such as autoimmune diseases and inflammatory diseases.

A first example of strategy for antagonizing IgG Fc binding to FcRn implies blocking antibodies directed against to FcRn (see e.g WO2002/43658, WO2018/083122, WO2020/079086). More specifically, several molecules based on this strategy are under clinical development. For instance, Rozanolixizumab (UCB7665) is a monoclonal antibody directed against FcRn developed by UCB for the treatment of chronic inflammatory demyelinating polyradiculoneuropathy, myasthenia gravis, and primary immune thrombocytopenia. Nipocalimab is another example pf anti-FcRn monoclonal antibody developed by Janssen. Other antibodies can also be cited such as IMVT-1401 and Orilanolimab.

A second example of strategy implies Fc fragment with an increased affinity for FcRn competing with IgG to occupy FcRn and thereby reducing the overall IgG recycling. Efgartigimod is an illustration of this second strategy and is developed by Argenx for myasthenia gravis, primary immune thrombocytopenia, pemphigus vulgaris and foliaceus, chronic inflammatory demyelinating polyradiculoneuropathy, bullous pemphigoid, and idiopathic inflammatory myopathy. Multimeric Fc molecules have also be described such as CSL730 developed by CSL/Momentas. For instance, the Fc region can be modified for increasing the affinity for FcRn and/or reducing pH dependence in comparison to a native Fc region (e.g., WO2015/100299).

ABY-039 is a peptide that specifically binds to FcRn fused to an albumin binding domain.

In addition, full-length IgG antibodies comprising variant Fc receptors with enhanced FcRn binding and decreased pH dependence have also been identified that antagonize FcRn binding to IgG (see e.g. WO2013/096221).

However, these strategies are not specific a particular antibody. It reduces the IgG level of all specificities, including protective antibodies. Therefore, they can lead to immunodeficiency.

Finally, a class of engineered antibody-based reagents called Seldegs have been developed for inducing a selective degradation of antigen-specific antibodies (Devanaboyina et al, 2017, Nat Commun., 8, 15314; WO2018/102668; Sun et al, 2021, Mol Ther, 29, 1312-1323). Seldeg molecules comprise a Fc region fused to an antigen. They have to be prepared with modified Fc region for modulating the capacity of the Fc region to bind FcRn (affinity and pH dependence). Indeed, in absence of the FcRn-enhancing mutation, the Seldeg molecule has no effect on the antibodies clearance. Then, it seems that the Seldeg technology requires a deep expertise for tuning the affinity and pH dependence of the interaction between FcRn and the Fc region of the Seldeg molecules. In addition, Seldeg mechanism involves an interaction with the endogenous FcRn and could have an impact on the overall mechanism of IgG recycling.

Then, there is still a need in the art for molecules that selectively deplete antigen-specific antibodies.

### SUMMARY OF THE INVENTION

The present invention provides with a new class of molecules suitable for selective clearance of specific antibodies directed against a particular antigen.

This new class of molecules is clearly distinct from known molecules previously described for antibodies clearance. The molecules comprise two covalently linked moieties: a moiety including the antigen for which a targeted antibody has a specificity; and another moiety being able to bind the targeted antibody, more specifically the Fc region of the targeted antibody. In particular, the molecules do not include any Fc region and do not bind FcRn. In a particular aspect, the moiety being able to bind the Fc region of the targeted antibody comprises the extracellular part of the FcRn and the beta-2 microglobulin. This is a clear difference compared to the molecules of the prior art.

The mechanism of this new class of molecules is the following and is illustrated in FIG. 19. In the extracellular compartment, the molecules specifically binds the targeted antibody by the interaction between the antigen moiety of the molecules and the antigen binding domain of the targeted antibody.

No binding at a blood physiological pH (e.g. a pH of about 7) is required between the molecules and the Fc region of the targeted antibody. After internalization in the lysosome, the pH is decreased and the molecule binds the Fc region of the targeted antibody. Thus, the Fc region of the targeted antibody is unavailable for an interaction of the endogenous FcRn and the targeted antibody is degraded and not recycled in the extracellular compartment. This mechanism allows the specific clearance of the targeted antibodies with no effect on the other antibodies.

Compared to other strategies, the present molecules present several advantages.

Compared to antibodies directed against FcRn or the molecules based on Fc region, the molecules of the present invention are specific for the clearance of antibodies directed against one particular antigen. In addition, they do not interfere with recycling of IgG because they do not bind endogenous FcRn.

Compared to Seldegs molecules, they do not interfere with recycling of IgG because they do not bind FcRn. In addition, the Seldeg strategy involves the interaction of three distinct partners, namely the targeted antibody, the Seldeg molecule and the endogenous FcRn, whereas the molecules of the present invention are based on a simpler and direct interaction between the targeted antibody and the molecules of the present invention, without any intervention of the endogenous FcRn.

Accordingly, the present invention relates to a molecule for selective clearance of an antibody directed against an antigen, wherein the molecule comprises
an extracellular part of a human neonatal Fc receptor (FcRn) including regions alpha1, alpha2 and alpha3 and devoid of transmembrane domain and
a beta-2 microglobulin;
said extracellular part of FcRn and/or said beta-2 microglobulin being covalently linked to the antigen of the antibody to be depleted or a fragment of said antigen which can be bound by the antibody to be depleted.

In a first aspect, the molecule comprises a single polypeptide chain comprising the extracellular part of FcRn, the beta-2 microglobulin and the antigen or the fragment thereof.

Preferably, the molecule comprises, from the N terminus to the C terminus, the beta-2 microglobulin, the region alpha1, the region alpha2, the region alpha3 and the antigen or the fragment thereof.

In a second aspect, the molecule comprises two polypeptide chains, a first polypeptide chain comprising the extracellular part of FcRn and a second polypeptide chain comprising the beta-2 microglobulin, and the antigen or the fragment thereof is covalently linked to the first polypeptide chain, the second polypeptide chain or both.

More specifically, the first polypeptide chain may comprise, from the N terminus to the C terminus, the antigen or the fragment thereof, the region alpha1, the region alpha2 and the region alpha3.

Alternatively, the first polypeptide chain may comprise, from the N terminus to the C terminus, the region alpha1, the region alpha2, the region alpha3 and the antigen or the fragment thereof.

Alternatively or in addition, the second polypeptide chain may comprise, from the N terminus to the C terminus, the antigen or the fragment thereof and the beta-2 microglobulin; or the beta-2 microglobulin and the antigen or the fragment thereof.

Those different aspects can be combined for in a molecule according to the present invention.

Optionally, the molecule may include several antigens or fragments thereof. The several antigens or fragments thereof can be identical or different. In a particular aspect, the antigens are different so as to deplete different antigen specific antibodies. For instance, the molecule can comprise a first antigen and a second antigen. Accordingly, the molecule comprises a first antigen or fragment thereof that can be bound by a first antibody to be depleted, and a second antigen or fragment thereof that can be bound by a second antibody to be depleted.

Preferably, the molecule binds human fragment crystallizable region (Fc region) of the antibody at endosomal pH, more specifically early endosomal pH, for instance pH from 5.5 to 6.5, but not at blood physiological pH, for instance pH from 7 to 7.5.

Optionally, the antibody binds the antigen or the fragment thereof of the molecule at blood physiological pH, for instance at pH from 7 to 7.5, and optionally at endosomal pH, more specifically early endosomal pH, for instance pH from 5.5 to pH 6.5.

Optionally, the antigen is an antigen inducing auto-antibody. Optionally, the antigen is an antigen inducing antibodies mediating a disease, especially an autoimmune disease, or a transplant rejection. Optionally, the antigen is recognized by an antibody used in diagnostic imaging.

For instance, the antigen can be selected from the group consisting of 60 kDa SS-A/Ro ribonucleoprotein, antigen La, a double-stranded DNA, histone, snRNP core protein, glycoprotein IIb, glycoprotein Illa, glycoprotein Ib, glycoprotein IX, neurofascin 155, contactin 1, Topoisomerase I, centromere, histidine-tRNA ligase, sp100 nuclear antigen, nucleoporin 210 kDa, actin, cyclic citrullinated peptide, myeloperoxidase, proteinase 3, cardiolipin, carbamylated protein, phospholipid, collagen, especially, collagen type IV alpha-3, thrombin, nicotinic acetylcholine receptor, muscle-specific kinase, voltage-gated calcium channel (P/Q-type), vinculin, thyroid peroxidase, thyroglobulin, thyrotropin receptor, neuronal nuclear protein, glutamate receptor, amphiphysin, glutamate decarboxylase, voltage-gated potassium channel, collapsin response mediator protein 5, N-methyl-D-aspartate receptor, aquaporin-4, desmoglein 3, desmoglein 1, phospholipase A2 receptor, myelin oligodendrocyte glycoprotein (MOG), myelin basic protein, proteolipid protein, myelin-associated glycoprotein, myelin-associated oligodendrocyte basic protein, transaldolase, low density lipoprotein receptor related protein 4, insulin, islet antigen 2, glutamic acid decarboxylase 65, zinc transporter 8, cartilage gp39, gp130-RAPS, 65 kDa heat shock protein, fibrillarin, small nuclear protein (snoRNP), thyroid stimulating factor receptor, nuclear antigens, glycoprotein gp70, ribosomes, pyruvate dehydrogenase dehydrolioamide acetyltransferase, hair follicle antigens, human tropomyosin isoform 5, a-amino-3-hydroxy-5-methyl-4-isoxazolepropionic acid (AMP A) receptor, GABAA and GABAB receptors, glycine receptor, and dipeptidyl-peptidase-like protein 6 (DPPX), more specifically selected from the group consisting of 60 kDa SS-A/Ro ribonucleoprotein, antigen La, a double-stranded DNA, histone, snRNP core protein, glycoprotein IIb, glycoprotein Illa, glycoprotein Ib, glycoprotein IX, neurofascin 155, contactin 1, Topoisomerase I, centromere, histidine-tRNA ligase, sp100 nuclear antigen, nucleoporin 210 kDa, actin, cyclic citrullinated peptide, myeloperoxidase, proteinase 3, cardiolipin, carbamylated protein, phospholipid, collagen type IV alpha-3, thrombin, nicotinic acetylcholine receptor, muscle-specific kinase, voltage-gated calcium channel (P/Q-type), vinculin, thyroid peroxidase, thyroglobulin, thyrotropin receptor, neuronal nuclear protein, glutamate receptor, amphiphysin, glutamate decarboxylase, voltage-gated potassium channel, collapsin response mediator protein 5, N-methyl-D-aspartate receptor, aquaporin-4, desmoglein 3, desmoglein 1, and phospholipase A2 receptor.

More specifically, the antigen can be selected from the group consisting of nicotinic acetylcholine receptor, muscle-specific kinase, desmoglein 3, desmoglein 1, glycoprotein IIb, glycoprotein Illa, glycoprotein Ib, glycoprotein IX, thyrotropin receptor, thyroid peroxidase, snRNP core protein, histone, antigen La and 60 kDa SS-A/Ro ribonucleoprotein.

Optionally, the extracellular part of FcRn can be modified for preventing or reducing the binding to albumin and/or fibrinogen. Optionally, said variant may comprise at least one mutation, preferably for preventing or reducing the binding to albumin. The mutation can be selected from the group consisting of a substitution of one amino acid W51, W53, W59, W61, or H166 by any other amino acid, preferably a substitution selected from the group consisting of W51A, W53A, W59A, W61A, H166A and any combination thereof, wherein the position of the amino acids correspond to the sequence as shown in SEQ ID NO: 2.

The present invention further relates to a pharmaceutical composition comprising a molecule as described herein or a nucleic acid or set of nucleic acids encoding said molecule. The present invention also relates to said molecule or pharmaceutical composition for its use as a drug, in particular for the treatment of an autoimmune disease, an inflammatory disease or disorder, or a transplant rejection, preferably an autoimmune disease. It relates to the use of said molecule or pharmaceutical composition for the manufacture of a drug, in particular for the treatment of an autoimmune disease, an inflammatory disease or disorder, or a transplant rejection, preferably an autoimmune disease. It relates to a method for treating a disease in a subject, comprising administering a therapeutic effective amount of said molecule or pharmaceutical composition to the subject. In particular, the disease is an autoimmune disease, an inflammatory disease or disorder, or a transplant rejection, preferably an autoimmune disease. More generally, the disease or disorder is mediated by an antibody or an excessive amount of antibody, the antibody being preferably specific of an auto-antigen.

In a very specific aspect, the disease to be treated is selected in the group consisting of Myasthenia Gravis, Pemphigus vulgaris, systemic lupus erythematosus, Sjögren's syndrome, immune thrombocytopenia (especially persistent or chronic immune thrombocytopenia), chronic inflammatory demyelinating polyneuropathy, scleroderma, CREST syndrome, inflammatory myopathy, primary biliary cirrhosis, coeliac disease, rheumatoid arthritis, granulomatosis, antiphospholipid syndrome, Goodpasture syndrome, chronic autoimmune hepatitis, polymyositis, small intestinal bacterial overgrowth, Hashimoto's thyroiditis, Graves' disease, paraneoplastic cerebellar degeneration, limbic encephalitis, encephalomyelitis, subacute sensory neuronopathy, choreoathetosis, opsoclonus myoclonus syndrome, Stiff-Person syndrome, diabetes mellitus type 1, Isaac's syndrome, optic neuropathy, anti-N-Methyl-D-Aspartate Receptor Encephalitis, neuromyelitis optica, Bullous pemphigoid, membranous nephropathy, allogenic islet graft rejection, alopecia areata, ankylosing spondylitis, autoimmune Addison's disease, Alzheimer's disease, antineutrophil cytoplasmic autoantibodies (ANCA), autoimmune diseases of the adrenal gland, autoimmune hemolytic anemia, autoimmune myocarditis, autoimmune neutropenia, autoimmune oophoritis and orchitis, autoimmune urticaria, Behcet's disease, cardiomyopathy, Castleman's syndrome, celiac spruce-dermatitis, chronic fatigue immune disfunction syndrome, Churg-Strauss syndrome, cicatrical pemphigoid, CREST syndrome, cold agglutinin disease, Crohn's disease, dermatomyositis, discoid lupus, epidermolysis bullosa acquisita, essential mixed cryoglobulinemia, fibromyalgia-fibromyositis, glomerulonephritis, Guillain-Barre syndrome, graft-versus-host disease (GVHD), hemophilia A, idiopathic membranous neuropathy, idiopathic pulmonary fibrosis, idiopathic thrombocytopenia purpura (ITP), IgA neuropathy, IgM polyneuropathies, juvenile arthritis, Kawasaki's disease, lichen plantus, lichen sclerosus, Meniere's disease, mixed connective tissue disease, mucous membrane pemphigoid, multiple sclerosis, type 1 diabetes mellitus, Multifocal motor neuropathy (MMN), pemphigoid gestationis, pemphigus foliaceus, pernicious anemia, polyarteritis nodosa, polychrondritis, polyglandular syndromes, polymyalgia rheumatica, primary agammaglobulinemia, psoriasis, psoriatic arthritis, relapsing polychondritis, Reynauld's phenomenon, Reiter's syndrome, sarcoidosis, solid organ transplant rejection, Takayasu arteritis, toxic epidermal necrolysis (TEN), Stevens Johnson syndrome (SJS), temporal arteristis/giant cell arteritis, thrombotic thrombocytopenia purpura, ulcerative colitis, uveitis, dermatitis herpetiformis vasculitis, anti-neutrophil cytoplasmic antibody-associated vasculitides, vitiligo, asthma, autoimmune pancreatitis, IgA nephropathy and Wegner's granulomatosis; optionally selected in the group consisting of Myasthenia Gravis, Pemphigus vulgaris, systemic lupus erythematosus, Sjögren's syndrome, immune thrombocytopenia (especially persistent or chronic immune thrombocytopenia), chronic inflammatory demyelinating polyneuropathy, scleroderma, CREST syndrome, inflammatory myopathy, primary biliary cirrhosis, coeliac disease, rheumatoid arthritis, granulomatosis, antiphospholipid syndrome, Goodpasture syndrome, chronic autoimmune hepatitis, polymyositis, small intestinal bacterial overgrowth, Hashimoto's thyroiditis, Graves' disease, paraneoplastic cerebellar degeneration, limbic encephalitis, encephalomyelitis, subacute sensory neuronopathy, choreoathetosis, opsoclonus myoclonus syndrome, Stiff-Person syndrome, diabetes mellitus type 1, Isaac's syndrome, optic neuropathy, anti-N-Methyl-D-Aspartate Receptor Encephalitis, neuromyelitis optica, Bullous pemphigoid, and membranous nephropathy, preferably selected in the group consisting of Myasthenia Gravis, Pemphigus vulgaris, systemic lupus erythematosus, Sjögren's syndrome, antiphospholipid syndrome, Hashimoto's thyroiditis and Graves' disease.

### BRIEF DESCRIPTION OF THE FIGURES

FIG. 1: Schematic representation of the FcRn molecules

FIG. 2: Pharmacokinetics of FcRn molecules in mice: 6 weeks old Balb/c mice were intraperitoneally injected with one dose with FcRn molecules. Concentration of the molecule in the sera was assessed by ELISA at multiple time points following injection. Injection with β2m-hFcRn-SIRPα-004 (100 μg) (▴), and SIRPα-FcSeldeg (100 μg) ( [symbol] ).

FIG. 3: Pharmacokinetics of anti-SIRPα antibody in mice in presence of FcRn molecules: 6 weeks old Balb/c mice were intraperitoneally injected with one dose of anti SIRPα antibody at day −1 (25 ug) and several doses of FcRn molecules at day 0, day 0+4h, day 0+8h, day 1, day 1+4h, day 1+8h, day 2, day 2+4h, day 2+8h (100 ug). Concentration of the molecule in the sera was assessed by ELISA at multiple time points following injection. Injection with anti SIRPα antibody alone ( [symbol] ), anti SIRPα antibody+β2m-hFcRn-SIRPα-004 (▴), anti SIRPα antibody+SIRPα-FcSeldeg (100 μg) ( [symbol] ).

FIG. 4: Pharmacokinetics of anti SIRPα antibody (A) and anti IL7Rα antibody (B) in mice: 6 weeks old Balb/c mice were intraperitoneally co-injected with one dose of anti SIRPα antibody and anti IL7Rα antibody at day 0 (25 ug) and several doses of FcRn molecules at day 1, day 1+4h, day 1+8h, day 2, day 2+4h, day 2+8h (100 ug). Concentration of the molecule in the sera was assessed by ELISA at multiple time points following injection. Injection with anti Sirpa antibody and anti IL7Rα antibody ( [symbol] ), anti Sirpa antibody and anti IL7Rα antibody+β2m-hFcRn-SIRPα-004 (100 μg) (▴), anti Sirpa antibody and anti IL7Rα antibody+SIRPα-FcSeldeg (100 μg) ( [symbol] ).

FIG. 5: Pharmacokinetics of anti SIRPα antibody in mice in presence of ascending doses of β2m-hFcRn-SIRPα-004: 6 weeks old Balb/c mice were intraperitoneally injected with one doses of anti SIRPα antibody at day 0 (25 μg) and one dose of β2m-hFcRn-SIRPα-004 at day 1 or two doses at day 1 and day 1+4h or three doses at day 1 and day 1+4 h and day 1+8h. Concentration of the molecule in the sera was assessed by ELISA at multiple time points following injection. Injection with anti SIRPα antibody alone ( [symbol] ), anti SIRPα antibody+one injection of β2m-hFcRn-SIRPα-004 (▴), anti SIRPα antibody+two injections of β2m-hFcRn-SIRPα-004 (▴), anti SIRPα antibody+three injections of β2m-hFcRn-SIRPα-004 ( [symbol] ). Intraperitoneal injections were realized at 30 μg, 100 μg and 300 μg.

FIG. 6: Pharmacokinetics of anti SIRPα antibody in mice in presence of FcRn molecules: 6 weeks old Balb/c mice were intraperitoneally injected with one dose of anti SIRPα antibody at day 0 (25 μg) and two doses of FcRn molecules at day 1 and day 1+4h (300 μg). Concentration of the molecule in the sera was assessed by ELISA at multiple time points following rejection. Injection with anti SIRPα antibody ( [symbol] ), anti SIRPα antibody+Sirpa-hFcRn/β₂m-001 ( [symbol] ), anti SIRPα antibody+hFcRn-Sirpa/β2m-002 ( [symbol] ), anti SIRPα antibody+β2m-hFcRn-Sirpa-004 (▴).

FIG. 7: Pharmacokinetics of anti SIRPα antibody and anti IL-7Rα antibody in mice in presence of FcRn molecules: 7 weeks old Balb/c mice were intraperitoneally co-injected with one dose of anti-SIRPα antibody and anti IL-7Rα antibody at day 0 (25 μg) and two doses of FcRn molecules at day 1 and day 2 (100 μg). Concentration of the molecule in the sera was assessed by ELISA at multiple time points following injection. Injection with anti SIRPα antibody and anti IL-7Rα antibody ( [symbol] ), anti SIRPα antibody and anti IL-7Rα antibody+β2m-hFcRn-Sirpa-004 (▴), anti SIRPα antibody and anti IL-7Rα antibody+β2m-hFcRn-IL7Rα-004 (●), anti SIRPα antibody and anti IL-7Rα antibody+β2m-hFcRn-Sirpa-004+β2m-hFcRn-IL7Rα-004 (♦).

FIG. 8: Pharmacokinetics of anti SIRPα antibody and anti IL-7Rα antibody in mice in presence of FcRn mutated molecules: 7 weeks old Balb/c mice were intraperitoneally co-injected with one dose of anti SIRPα antibody and anti IL-7Rα antibody at day 0 (25 μg) and one dose of FcRn mutated molecules at day 1 (100 μg or 300 μg). Concentration of the molecule in the sera was assessed by ELISA at multiple time points following injection. Injection with anti SIRPα antibody and anti IL-7Rα antibody alone ( [symbol] ), anti SIRPα antibody and anti IL-7Rα antibody+β2m-hFcRn-Sirpa-004 (300 μg) (▴), anti SIRPα antibody and anti IL-7Rα antibody+β2m-hFcRn (300 μg) (●), anti SIRPα antibody and anti IL-7Rα antibody+β2m-hFcRn-H166A-Sirpa-010 (100 μg) ( [symbol] ), anti SIRPα antibody and anti IL-7Rα antibody+β2m-hFcRn-W51A-Sirpa-011 (300 μg) (Δ), anti SIRPα antibody and anti IL-7Rα antibody+β2m-hFcRn-W53A-Sirpa-012 (300 μg) (▾), anti SIRPα antibody and anti IL-7Rα antibody+β2m-hFcRn-W59A-Sirpa-013 (300 μg) (∇), anti SIRPα antibody and anti IL-7Rα antibody+β2m-hFcRn-W61A-Sirpa-014 (300 μg) ( [symbol] ), anti SIRPα antibody and anti IL-7Rα antibody+β2m-hFcRn-IL7Rα-004 (300 μg) (▪) and anti SIRPα antibody and anti IL-7Rα antibody+ARGX113 (300 μg) (▪).

FIG. 9: kinetics of albumin concentration in mice: 7 weeks old Balb/c mice were intraperitoneally co-injected with one dose of anti SIRPα antibody and anti IL-7Rα antibody at day 0 (25 μg) and one dose of FcRn mutated molecules at day 1 (100 μg or 300 μg). Concentration of albumin in the sera was assessed by ELISA at multiple time points following injection. Injection with anti SIRPα antibody and anti IL-7Rα antibody alone ( [symbol] ), anti SIRPα antibody and anti IL-7Rα antibody+β2m-hFcRn-Sirpa-004 (300 μg) (▴), anti SIRPα antibody and anti IL-7Rα antibody+β2m-hFcRn (300 μg) (●), anti SIRPα antibody and anti IL-7Rα antibody+β2m-hFcRn-H166A-Sirpa-010 (100 μg) ( [symbol] ), anti SIRPα antibody and anti IL-7Rα antibody+β2m-hFcRn-W51A-Sirpa-011 (300 μg) (Δ), anti SIRPα antibody and anti IL-7Rα antibody+β2m-hFcRn-W53A-Sirpa-012 (300 μg) (▾), anti SIRPα antibody and anti IL-7Rα antibody+β2m-hFcRn-W59A-Sirpa-013 (300 μg) (∇), anti SIRPα antibody and anti IL-7Rα antibody+β2m-hFcRn-W61A-Sirpa-014 (300 μg) ( [symbol] ), anti SIRPα antibody and anti IL-7Rα antibody+β2m-hFcRn-IL7Rα-004 (300 μg) (▪) and anti SIRPα antibody and anti IL-7Rα antibody+ARGX113 (300 μg) (▪).

FIG. 10: Pharmacokinetics of FcRn mutated molecules in mice: 7 week old Balb/c mice were intraperitoneally co-injected with one dose of anti SIRPα antibody and anti IL-7Rα antibody at day −1 (25 μg) and one dose of FcRn mutated molecules at day 0 (300 μg). Concentration of the molecule in the sera was assessed by ELISA at multiple time points following injection. Injection with anti SIRPα antibody and anti IL-7Rα antibody+β2m-hFcRn-Sirpa-004 (▴), anti SIRPα antibody and anti IL-7Rα antibody+β2m-hFcRn (▪), anti SIRPα antibody and anti IL-7Rα antibody+β2m-hFcRn-H166A-Sirpa-010 (100 μg) ( [symbol] ), anti SIRPα antibody and anti IL-7Rα antibody+β2m-hFcRn-W51A-Sirpa-011 (Δ), anti SIRPα antibody and anti IL-7Rα antibody+β2m-hFcRn-W53A-Sirpa-012 (▾), anti SIRPα antibody and anti IL-7Rα antibody+β2m-hFcRn-W59A-Sirpa-013 (∇), anti SIRPα antibody and anti IL-7Rα antibody+β2m-hFcRn-W61A-Sirpa-014 ( [symbol] ).

FIG. 11: Pharmacokinetics of anti-SIRPα antibody and anti-IL7Rα antibody in NHP in presence of β2m-hFcRn-SIRPα-004: Two non-human primates were intravenously co-injected with one dose of anti-SIRPα antibody (▴) and anti-IL7Rα antibody ( [symbol] ) at day 0 at 1 mg/kg and one dose of β2m-hFcRn-Sirpa-004 at day 2 at 10 mg/kg. Pharmacokinetics of anti-SIRPα antibody and anti-IL7Rα antibody were evaluated by Elisa and graph represents normalized data to D2.

FIG. 12: Physiological parameters of NHP after intravenously injection of anti-SIRPα antibody and anti-IL7Rα antibody in presence of β2m-hFcRn-Sirpa-004: Two non-human primates were intravenously co-injected with one dose of anti-SIRPα antibody and anti-IL7Rα antibody at day 0 at 1 mg/kg and β2m-hFcRn-Sirpa-004 at day 2 at 10 mg/kg. Graph represents Temperature, Saturation of 02, Cardiac frequency and PAM of NHP (NHP-1: ▪ and NHP-2: ●).

FIG. 13: Concentration of proteins in sera of NHP after intravenously injection of anti-SIRPα antibody and anti-IL7Rα antibody in presence of β2m-hFcRn-Sirpa-004: Two non-human primates (NHP-1 (▾) and NHP-2 (●) were intravenously co-injected with anti-SIRPα antibody and anti-IL7Rα antibody at day 0 at 1 mg/kg and β2m-hFcRn-Sirpa-004 at day 2 at 10 mg/kg (NHP-1: ▪ and NHP-2: ●).

FIG. 14: Measurement of anti-RBD IgG titers on immunized mice balb/c model with peptide from viral RBD protein to induce humoral B cell response and treated with β2m-mFcRn-vRBD-004 molecules: (A) 6-weeks-old female balb/c mouse mice were subcutaneously immunized with two peptides designed to induce humoral B cell response in footpath (from RBD viral protein) at day 0 and 7 with 50 μg per injection. At day 37, after validation of anti-vRBD antibodies production by mice, they were injected with Mycophenolate mofetil at 50 mg/kg (▪), Mycophenolate mofetil at 50 mg/kg+β2m-mFcRn-vRBD-004 (4 mg/kg) (□), ARGX113 (4 mg/kg) (▴) or PBS ( [symbol] ) at D37, D39 and D41. (B) Mice were pretreated with Mycophenolate mofetil at 50 mg/kg (Δ) during several weeks and injected at D55 with PBS or β2m-mFcRn-vRBD-004 (12 mg/kg) (▴). (C) Mice were treated with Mycophenolate mofetil at 50 mg/kg and PBS (◯) or Mycophenolate mofetil at 50 mg/kg+β2m-mFcRn-vRBD-004 (12 mg/kg) (⊙) the same day. Anti-RBD IgG titers was measured by ELISA on RBD protein coated and graf represents the evolution of titers in function of timing.

FIG. 15: Pharmacokinetics of anti-SIRPα antibody and anti-IL7Rα antibody in NHP in presence of β2m-hFcRn-Sirpa-004: Two non-human primates were intravenously co-injected with anti-SIRPα antibody and anti-IL7Rα antibody at day −2 at 1 mg/kg and three injections of β2m-hFcRn-Sirpa-004 at day 0, 1 and 2 at 10 mg/kg (NHP-1 ( [symbol] ) and NHP-2 (▪). Pharmacokinetics of anti-SIRPα antibody and anti-IL7Rα antibody were evaluated by Elisa and graph represents concentration (ng/ml).

FIG. 16: Concentration of proteins in sera of NHP injected intravenously with one dose of anti-SIRPα antibody and anti-IL7Rα antibody and treated with three doses of β2m-hFcRn-Sirpa-004: Two non-human primates (NHP-1 ( [symbol] ) and NHP-2 (▪) were intravenously co-injected with one dose of anti-SIRPα antibody and anti-IL7Rα antibody at day −2 at 1 mg/kg and three doses of β2m-hFcRn-Sirpa-004 at day 0, 1 and 2 at 10 mg/kg.

FIG. 17: Temperature, saturation of 02, cardiac frequency and PAM of NHP injected intravenously with one dose of anti-SIRPα antibody and anti-IL7Rα antibody and treated with three doses of β2m-hFcRn-Sirpa-004 were represented. Two non-human primates were intravenously co-injected with one dose of anti-SIRPα antibody and anti-IL7Rα antibody at day −2 at 1 mg/kg and three doses of β2m-hFcRn-Sirpa-004 at day 0, 1 and 2 at 10 mg/kg (NHP-1 ( [symbol] ) and NHP-2 (▪).

FIG. 18: Pharmacokinetics of anti SIRPα antibody and anti IL7Rα antibody in mice: 7 weeks old Balb/c mice were intraperitoneally co-injected with one dose of anti-SIRPα antibody and anti-IL7Rα antibody at day 0 (25 μg) and one dose of bispecific FcRn molecule at day 1 (300 μg). Concentration of the molecule in the sera was assessed by ELISA at multiple time points following injection. Injection with anti Sirpa antibody and anti IL7Rα antibody ( [symbol] ), anti Sirpa antibody and anti IL7Rα antibody+IL-7Rα-hFcRn-SIRPα/β2m-023 (300 μg) (▾). Concentration (ng/ml) (A) and normalized data to day 1 were represented in graphs.

FIG. 19: Schema illustrating the antigen-specific antibody elimination.

FIG. 20: Pharmacokinetics of humanized anti-SIRPα antibody (A) and humanized anti-IL-7Rα antibody (B) in mice in presence of FcRn molecules. 7 weeks old Balb/c mice were intraperitoneally co-injected with one dose of humanized anti-SIRPα antibody and humanized anti-IL-7Rα antibody at day 0 (25 μg) and one dose of FcRn molecules at day 1 (300 μg). Concentration of the molecule in the sera was assessed by ELISA at multiple time points following injection. Injection with humanized anti-SIRPα antibody and humanized anti-IL-7Rα antibody ( [symbol] ), humanized anti-SIRPα antibody and humanized anti-IL-7Rα antibody+IL-7Rα-Sirpa-hFcRn/B2m-19 ( [symbol] ), humanized anti-SIRPα antibody and humanized anti-IL-7Rα antibody+hFcRn-IL-7Rα-Sirpa/B2m-21 ( [symbol] ), humanized anti-SIRPα antibody and humanized anti-IL-7Rα antibody+IL-7Rα-hFcRn-Sirpa/B2m-23 ( [symbol] ), humanized anti-SIRPα antibody and humanized anti-IL-7Rα antibody+hFcRn/B2m-IL-7Rα-Sirpa-25 ( [symbol] ), humanized anti-SIRPα antibody and humanized anti-IL-7Rα antibody+IL-7Rα-hFcRn/B2m-Sirpa-30 ( [symbol] ), humanized anti-SIRPα antibody and humanized anti-IL-7Rα antibody+B2m-hFcRn-IL-7Rα-Sirpa-31 ( [symbol] ), humanized anti-SIRPα antibody and humanized anti-IL-7Rα antibody+B2m-IL-7Rα-hFcRn-Sirpa-33 ( [symbol] ).

FIG. 21: Pharmacokinetics of humanized anti-SIRPα antibody (A) and humanized anti-IL-7Rα antibody (B) in mice in presence of FcRn molecules. 7 weeks old Balb/c mice were intraperitoneally co-injected with one dose of humanized anti-SIRPα antibody and humanized anti-IL-7Rα antibody at day 0 (25 μg) and one dose of FcRn molecules at day 1 (300 μg). Concentration of the molecule in the sera was assessed by ELISA at multiple time points following injection. Injection with humanized anti-SIRPα antibody and humanized anti-IL-7Rα antibody ( [symbol] ), humanized anti-SIRPα antibody and humanized anti-IL-7Rα antibody+hFcRn-SIRPα-IL7Rα/B2m-022 ( [symbol] ), humanized anti-SIRPα antibody and humanized anti-IL-7Rα antibody+B2m-hFcRn-SIRPα-IL7Rα-032 ( [symbol] ).

FIG. 22: Pharmacokinetics of mouse anti-vRBD antibody isolated from vRBD immunized mice in presence of β2m-msFcRn-vRBD molecules. 7 weeks old Balb/c mice were intraperitoneally injected with sera containing mouse anti-vRBD antibody at day 0 (149 μg (A), 29.8 μg (B) or 5.96 μg (C)) and two doses of FcRn molecules at day 1 and day 2 (500 μg). Concentration of the molecule in the sera was assessed by ELISA at multiple time points following injection. Injection with sera containing mouse anti-vRBD antibody ( [symbol] ), sera containing mouse anti-vRBD antibody and β2m-msFcRn-vRBD molecule ( [symbol] ) were represented.

FIG. 23: Pharmacokinetics of mouse anti-hDSG3 antibody isolated from hDSG3 immunized mice in presence of β2m-msFcRn-hDSG3 molecules. 7 weeks old Balb/c mice were intraperitoneally injected with sera mouse containing anti-hDSG3 antibody at day 0 (200l) and one doses of FcRn molecules at day 1 (1000 μg). Concentration of the molecule in the sera was assessed by ELISA at multiple time points following injection (Day 1 ( [symbol] ), Day 2 ( [symbol] ), Day3 ( [symbol] ). Injection with sera containing mouse anti-hDSG3 antibody (A) or sera containing mouse anti-hDSG3 antibody and β2m-msFcRn-hDSG3 molecule (B) were represented.

FIG. 24: Pharmacokinetics of anti-SIRPα antibody (A) and anti-IL7Rα antibody (B) in mice in presence of FcRn molecules. 7 weeks old Balb/c mice were intraperitoneally co-injected with one dose of anti-SIRPα antibody and anti-IL-7Rα antibody at day 0 (25 μg) and two doses of FcRn molecules at day 1 and 2 (500 μg). Concentration of the molecule in the sera was assessed by ELISA at multiple time points following injection. Injection with humanized anti-SIRPα IgG4 mutated (S228P) antibody+human anti-IL-7Rα antibody ( [symbol] ), humanized anti-SIRPα IgG4 mutated (S228P) antibody+human anti-IL-7Rα antibody+β2m-hFcRn-SIRPα (▪), humanized anti-SIRPα IgG1 mutated (E333A)+human anti-IL-7Rα antibody (+), humanized anti-SIRPα IgG1 mutated (E333A)+human anti-IL-7Rα antibody+β2m-hFcRn-SIRPα ( [symbol] ), human anti-SIRPα/γ IgG4mutated (S228P)+human anti-IL-7Rα antibody ( [symbol] ), human anti-SIRPα/γ IgG4mutated (S228P)+human anti-IL-7Rα antibody+R2m-hFcRn-SIRPα ( [symbol] ).

### DETAILED DESCRIPTION OF THE INVENTION

### Definition

In order that the present invention may be more readily understood, certain terms are defined hereafter. Additional definitions are set forth throughout the detailed description.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art.

As used herein, the “sequence identity” between two sequences is described by the parameter “sequence identity”, “sequence similarity” or “sequence homology”. For purposes of the present invention, the “percentage identity” between two sequences (A) and (B) is determined by comparing the two sequences aligned in an optimal manner, through a window of comparison. Said alignment of sequences can be carried out by well-known methods in the art, for example, using the algorithm for global alignment of Needleman-Wunsch. Protein analysis software matches similar sequences using measures of similarity assigned to various substitutions, deletions and other modifications, including conservative amino acid substitutions. Once the total alignment is obtained, the percentage of identity can be obtained by dividing the full number of identical amino acid residues aligned by the full number of residues contained in the longest sequence between the sequence (A) and (B). Sequence identity is typically determined using sequence analysis software. For comparing two amino acid sequences, one can use, for example, the tool “Emboss needle” for pairwise sequence alignment of proteins providing by EMBL-EBI and available on:

www.ebi.ac.uk/Tools/services/web/toolform.ebi?tool=emboss_needle&context=protein, for example using default settings: (I) Matrix: BLOSUM62, (ii) Gap open: 10, (iii) gap extend: 0.5, (iv) output format pair, (v) end gap penalty: false, (vi) end gap open: 10, (vii) end gap extend: 0.5.

Alternatively, Sequence identity can also be typically determined using sequence analysis software Clustal Omega using the HHalign algorithm and its default settings as its core alignment engine. The algorithm is described in Söding, J. (2005) ‘Protein homology detection by HMM-HMM comparison’. Bioinformatics 21, 951-960, with the default settings.

By “amino acid change” or “amino acid modification” is meant herein a change in the amino acid sequence of a polypeptide. “Amino acid modifications” include substitution, insertion and/or deletion in a polypeptide sequence. By “amino acid substitution” or “substitution” herein is meant the replacement of an amino acid at a particular position in a parent polypeptide sequence with another amino acid. By “amino acid insertion” or “insertion” is meant the addition of an amino acid at a particular position in a parent polypeptide sequence. By “amino acid deletion” or “deletion” is meant the removal of an amino acid at a particular position in a parent polypeptide sequence. The amino acid substitutions may be conservative. A conservative substitution is the replacement of a given amino acid residue by another residue having a side chain (“R-group”) with similar chemical properties (e.g., charge, bulk and/or hydrophobicity). As used herein, “amino acid position” or “amino acid position number” are used interchangeably and refer to the position of a particular amino acid in an amino acids sequence, generally specified with the one letter codes for the amino acids. The first amino acid in the amino acids sequence (i.e. starting from the N terminus) should be considered as having position 1.

As used herein, the term “antibody” describes a IgG type of immunoglobulin molecule and is used in its broadest sense. In particular, antibodies include IgG1, IgG2, IgG3, and IgG4 class. Preferably, the term antibody refers to a human antibody.

As used herein, an “antigen-binding domain” of an antibody means a part of an antibody, i.e. a molecule corresponding to a portion of the structure of the antibody of the invention, that exhibits antigen-binding capacity for a particular antigen, possibly in its native form. The antigen-binding capacity can be determined by measuring the affinity between the antibody and the target fragment (i.e., antigen or fragment thereof). Antigen-binding domain of antibodies comprises the hypervariable domains of the antibody or the 6 CDRs (Complementary Determining Regions) thereof.

As used herein, the terms “fragment crystallizable region” or “Fc region” or “Fc domain” are interchangeable and refers to the tail region of an antibody that interacts with cell surface receptors called Fc receptors. The Fc region or domain is typically composed of two identical domains, derived from the second and third constant domains of the antibody's two heavy chains (i.e. CH2 and CH3 domains). Optionally, the Fc domain is that from IgG1, IgG2, IgG3 or IgG4, optionally with IgG1 hinge-CH2-CH3 and IgG4 hinge-CH2-CH3. Optionally, the Fc domain is a human Fc domain.

The term “antigen-specific antibody” as used herein refers to an antibody or antibody that binds to a particular antigen or antigen fragment.

The term “antigen fragment” as used herein refers to a part of the antigen that can be recognized by the antigen-specific antibody.

By “endogenous” FcRn, it is referred to the FcRn naturally present at the cell surface.

As used herein, a “pharmaceutical composition” refers to a preparation of one or more of the active agents, such as comprising a molecule according to the invention, with optional other chemical components such as physiologically suitable carriers and excipients. The purpose of a pharmaceutical composition is to facilitate administration of the active agent to an organism. Compositions of the present invention can be in a form suitable for any conventional route of administration or use. In one embodiment, a “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. An “acceptable vehicle” or “acceptable carrier” as referred to herein, is any known compound or combination of compounds that are known to those skilled in the art to be useful in formulating pharmaceutical compositions.

“An effective amount” or a “therapeutic effective amount” as used herein refers to the amount of active agent required to confer therapeutic effect on the subject, either alone or in combination with one or more other active agents, e.g. the amount of active agent that is needed to treat the targeted disease or disorder, or to produce the desired effect. The “effective amount” will vary depending on the agent(s), the disease and its severity, the characteristics of the subject to be treated including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

As used herein, the term “medicament” refers to any substance or composition with curative or preventive properties against disorders or diseases.

The term “treatment” refers to any act intended to ameliorate the health status of patients such as therapy, prevention, prophylaxis and retardation of the disease or of the symptoms of the disease. It designates both a curative treatment and/or a prophylactic treatment of a disease. A curative treatment is defined as a treatment resulting in cure or a treatment alleviating, improving and/or eliminating, reducing and/or stabilizing a disease or the symptoms of a disease or the suffering that it causes directly or indirectly. A prophylactic treatment comprises both a treatment resulting in the prevention of a disease and a treatment reducing and/or delaying the progression and/or the incidence of a disease or the risk of its occurrence. In certain embodiments, such a term refers to the improvement or eradication of a disease, a disorder, an infection or symptoms associated with it. Treatments according to the present invention do not necessarily imply 100% or complete treatment. Rather, there are varying degrees of treatment of which one of ordinary skill in the art recognizes as having a potential benefit or therapeutic effect. Preferably, the term “treatment” refers to the application or administration of a composition including one or more active agents to a subject who has a disorder/disease.

As used herein, the terms “disorder” or “disease” refer to the incorrectly functioning organ, part, structure, or system of the body resulting from the effect of genetic or developmental errors, infection, poisons, nutritional deficiency or imbalance, toxicity, or unfavorable environmental factors. Preferably, these terms refer to a health disorder or disease e.g. an illness that disrupts normal physical or mental functions.

As used herein, the term “isolated” indicates that the recited material (e.g., antibody, polypeptide, nucleic acid, etc.) is substantially separated from, or enriched relative to, other materials with which it occurs in nature. Particularly, an “isolated” molecule is one which has been identified and separated and/or recovered from a component of its natural environment.

The term “and/or” as used herein is to be taken as specific disclosure of each of the two specified features or components with or without the other. For example, “A and/or B” is to be taken as specific disclosure of each of (i) A, (ii) B and (iii) A and B, just as if each is set out individually.

The term “a” or “an” can refer to one of or a plurality of the elements it modifies (e.g., “a reagent” can mean one or more reagents) unless it is contextually clear either one of the elements or more than one of the elements is described.

The term “about” as used herein in connection with any and all values (including lower and upper ends of numerical ranges) means any value having an acceptable range of deviation of up to +/−10% (e.g., +/−0.5%, +/−1%, +/−1.5%, +/−2%, +/−2.5%, +/−3%, +/−3.5%, +/−4%, +/−4.5%, +/−5%, +/−5.5%, +/−6%, +/−6.5%, +/−7%, +/−7.5%, +/−8%, +/−8.5%, +/−9%, +/−9.5%). The use of the term “about” at the beginning of a string of values modifies each of the values (i.e. “about 1, 2 and 3” refers to about 1, about 2 and about 3). Further, when a listing of values is described herein (e.g. about 50%, 60%, 70%, 80%, 85% or 86%) the listing includes all intermediate and fractional values thereof (e.g., 54%, 85.4%).

### Molecules

The present invention relates to a new class of molecules for selective clearance of a targeted antibody directed against an antigen.

The molecules comprises two covalently linked moieties: a moiety including the antigen for which a targeted antibody has a specificity; and another moiety being able to bind the targeted antibody, more specifically the Fc region of the targeted antibody. In particular, the molecules do not include any Fc region and do not bind FcRn. In a particular aspect, the moiety being able to bind the Fc region of the targeted antibody comprises the extracellular part of the FcRn and the beta-2 microglobulin.

The mechanism of this new class of molecules is highly innovative and clearly distinct from the strategies for antibodies clearance known in the art as illustrated by FIG. 19. In the extracellular compartment, the molecules specifically binds the targeted antibody by the interaction between the antigen moiety of the molecules and the antigen binding domain of the targeted antibody. No binding at blood physiological pH, for instance at pH from 7 to 7.5, is required between the molecules and the Fc region of the targeted antibody. After internalization in the lysosome, the pH is decreased and the molecule binds the Fc region of the targeted antibody. Thus, the Fc region of the targeted antibody is unavailable for an interaction of the endogenous FcRn and the targeted antibody is degraded and not recycled at the extracellular compartment. This mechanism allows the specific clearance of the targeted antibodies with no effect on the other antibodies or impact on the IgG recycling process.

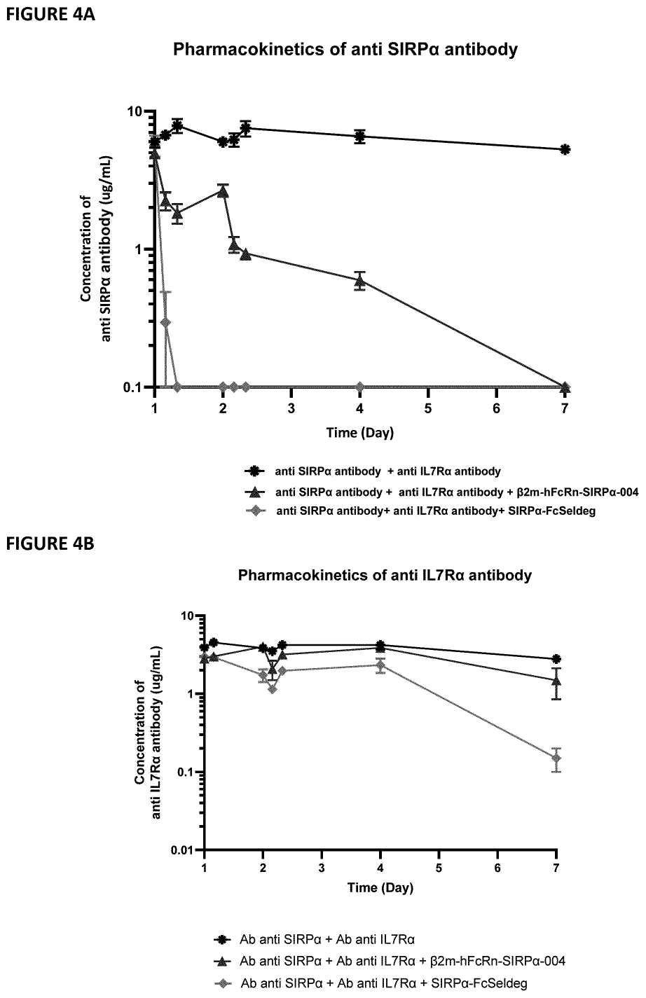

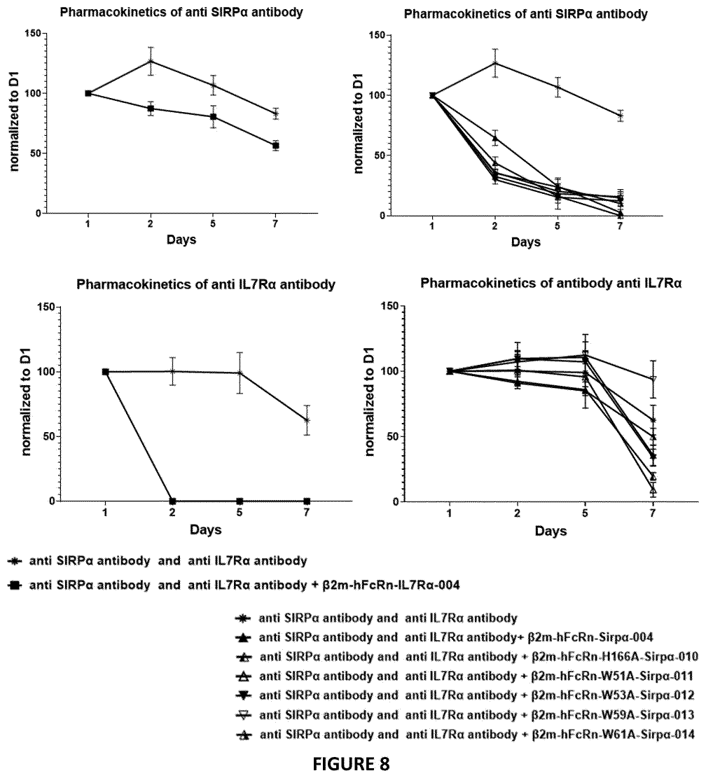

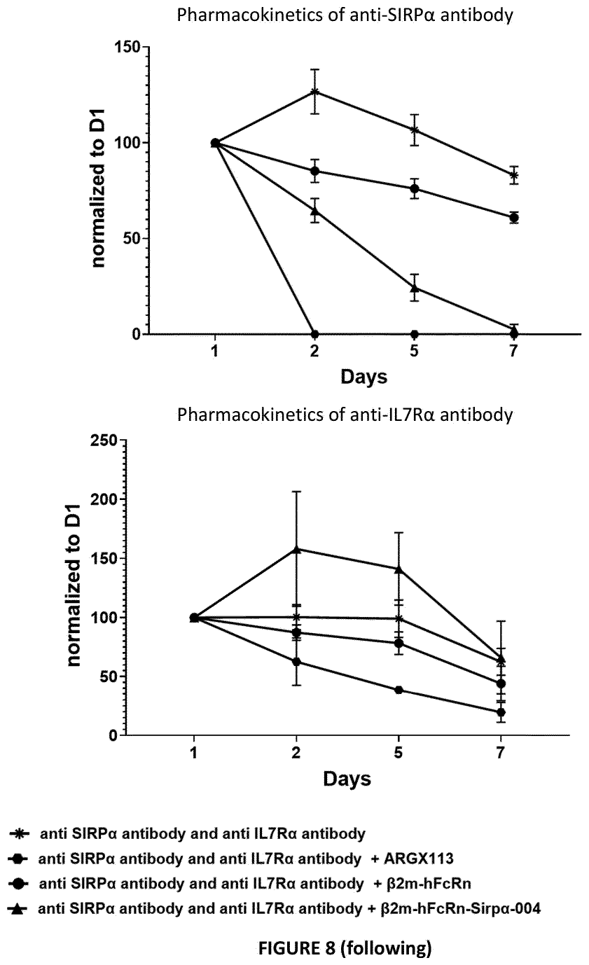

As demonstrated in the examples, this molecule is capable of selectively depleting the antibody specific for the antigen included in the molecules, without any impact on the other immunoglobulins, including the IgGs, IgAs and IgM. The clearance effect of the molecule is then highly specific of the targeted antibody. In comparison to Seldeg molecules, the molecules of the present invention present a better depletion specificity (see, FIG. 4B). The same advantageous specificity has been observed in comparison to the reference molecule ARGX113 (see, FIG. 8).

The mechanism of action of the new molecules is different from antibodies directed against FcRn, molecules having Fc region with high affinity for FcRn or Seldeg molecules. Indeed, the effect is not based on any competition with the Fc/FcRn interaction. The new molecules do not comprise any Fc region and do not interact with FcRn, especially the endogenous FcRn.

The targeted antibodies are IgG antibodies and present a Fc region and two antigen binding domains. In a specific aspect, the IgG antibodies are human IgG. Alternatively, if the subject to be treated is an animal, the IgG antibodies can be an animal IgG.

### Moiety Binding to the Antibody Fc Region

The molecule comprises a moiety being able to bind the targeted antibody, more specifically the Fc region of the targeted antibody. In a particular aspect, this moiety comprises the extracellular part of the FcRn and the beta-2 microglobulin.

As used herein, the terms “FcRn” refers to the neonatal Fc receptor, IgG receptor FcRn large subunit p51 or IgG Fc fragment receptor transporter alpha chain. The protein is encoded in humans by the FCGRT gene. In a preferred aspect, the FcRn is a human FcRn. For example, the human FcRn amino acid sequence has a Genbank accession number of NP_001129491.1 or NP_004098.1. Human FcRn is for example described in UniProtKB—P55899. The human FcRn amino acid sequence is about 365 amino acids. The extracellular domain of FcRn is from position 24 to position 297, the transmembrane domain is from position 298 to position 321 and the cytoplasmic domain is from position 322 to 365. The alpha1 region of FcRn is from position 24 to position 110. The alpha2 region of FcRn is from position 111 to position 200. The alpha3 region of FcRn is from position 201 to position 290.

```
FcRn amino acid sequence
SEQ ID NO: 1
                10 20 30 40 50
MGVPRPQPWA LGLLLFLLPG SLGAESHLSL LYHLTAVSSP APGTPAFWVS
                60 70 80 90 100
GWLGPQQYLS YNSLRGEAEP CGAWVWENQV SWYWEKETTD LRIKEKLFLE
              110 120 130 140 150
AFKALGGKGP YTLQGLLGCE LGPDNTSVPT AKFALNGEEF MNFDLKQGTW
              160 170 180 190 200
GGDWPEALAI SQRWQQQDKA ANKELTELLF SCPHRLREHL ERGRGNLEWK
              210 220 230 240 250
EPPSMRLKAR PSSPGFSVLT CSAFSFYPPE LQLRFLRNGL AAGTGQGDFG
              260 270 280 290 300
PNSDGSFHAS SSLTVKSGDE HHYCCIVQHA GLAQPLRVEL ESPAKSSVLV
              310 320 330 340 350
VGIVIGVLLL TAAAVGGALL WRRMRSGLPA PWISLRGDDT GVLLPTPGEA
              360
QDADLKDVNV IPATA
```

Then, the molecule comprises the extracellular part of FcRn, especially a human FcRn, including regions alpha1, alpha2 and alpha3. For instance, the molecule comprises the sequence from the position 24 to the position 290 of SEQ ID NO: 1 or a sequence having at least 80, 85, 90, 95, 96, 97, 98 or 99% of identity with the sequence from the position 24 to the position 290 of SEQ ID NO: 1. Optionally, the extracellular part of FcRn of the molecule includes the sequence from the position 24 to the position 290 of SEQ ID NO: 1 or a sequence having at least 80, 85, 90, 95, 96, 97, 98 or 99% of identity with the sequence from the position 24 to the position 290 of SEQ ID NO: 1. Optionally, it may include the extracellular domain of FcRn from position 24 to position 297 of SEQ ID NO: 1 or a sequence having at least 80, 85, 90, 95, 96, 97, 98 or 99% of identity therewith. The FcRn sequence included in the molecule is preferably without the signal peptide (from position 1 to position 23).

The molecule is soluble and do not bound to the membrane. Therefore, the molecule does not comprise any transmembrane domain, especially the FcRn transmembrane domain.

Optionally, the extracellular part of FcRn can be modified for preventing or reducing the binding to albumin and/or fibrinogen.

In a particular aspect, the modified extracellular part of FcRn variant is modified for preventing or reducing the binding to albumin. It may comprise one or several mutations. The mutation can be selected from the group consisting of a substitution of one amino acid W51, W53, W59, W61, or H166 by any other amino acid, preferably a substitution selected from the group consisting of W51A, W53A, W59A, W61A, H166A and any combination thereof, wherein the position of the amino acids correspond to the sequence as shown in SEQ ID NO: 2.

(hFcRn without signal peptide and including
regions alpha1, alpha2 and alpha3))
SEQ ID NO: 2
AESHLSLLYHLTAVSSPAPGTPAFWVSGWLGPQQYLSYNSLRGEAEPCG
AWVWENQVSWYWEKETTDLRIKEKLFLEAFKALGGKGPYTLQGLLGCEL
GPDNTSVPTAKFALNGEEFMNFDLKQGTWGGDWPEALAISQRWQQQDKA
ANKELTFLLFSCPHRLREHLERGRGNLEWKEPPSMRLKARPSSPGFSVL
TCSAFSFYPPELQLRFLRNGLAAGTGQGDFGPNSDGSFHASSSLTVKSG
DEHHYCCIVQHAGLAQPLRVELESPAKSS

The moiety being able to bind the targeted antibody comprises, in addition to the extracellular part of FcRn, a beta-2 microglobulin. Preferably, the beta-2 microglobulin is the human beta-2 microglobulin. The protein is encoded in humans by the B2M gene. For example, the human beta-2 microglobulin amino acid sequence has a Genbank accession number of NP_004039. Human beta-2 microglobulin is for example described in UniProtKB—P61769. The human beta-2 microglobulin amino acid sequence is about 119 with a signal peptide from position 1 to position 20. The beta-2 microglobulin sequence included in the molecule is preferably without the signal peptide.

```
human beta-2 microglobulin
SEQ ID NO: 3
                10 20 30 40 50
MSRSVALAVL ALLSLSGLEA IQRTPKIQVY SRHPAENGKS NFLNCYVSGF
                60 70 80 90 100
HPSDIEVDLL KNGERIEKVE HSDLSFSKDW SFYLLYYTEF TPTEKDEYAC
              110
RVNHVTLSQP KIVKWDRDM
```

Then, the molecule comprises the beta-2 microglobulin. For instance, the molecule comprises the sequence from the position 21 to the position 119 of SEQ ID NO: 3 (SEQ ID NO: 4) or a sequence having at least 80, 85, 90, 95, 96, 97, 98 or 99% of identity with the sequence from the position 21 to the position 119 of SEQ ID NO: 3.

In a preferred aspect, the molecule comprises an extracellular part of a human neonatal Fc receptor (FcRn) including regions alpha1, alpha2 and alpha3 and devoid of transmembrane domain, and a beta-2 microglobulin.

The molecule can be a polymeric protein, more specifically dimeric protein, with a first polypeptide chain comprising the extracellular part of FcRn as defined herein and with a second polypeptide chain comprising the beta-2 microglobulin as defined herein.

Alternatively, the molecule can be a single polypeptide chain in which the extracellular part of FcRn as defined herein is fused to the beta-2 microglobulin as defined herein. The protein fusion is carried out so as to allow the appropriate interaction of the alpha3 region of FcRn with the beta-2 microglobulin. Optionally, the extracellular part of FcRn and the beta-2 microglobulin are fused together through a peptide linker.

In a particular aspect, the molecule comprises, from the N terminus to the C terminus, the beta-2 microglobulin, the region alpha1, the region alpha2, and the region alpha3 of the FcRn. More particularly, it comprises from the N terminus to the C terminus, the beta-2 microglobulin, a peptide linker, the region alpha1, the region alpha2, and the region alpha3 of the FcRn.

As used herein, the term “linker” refers to a sequence that is useful to prevent steric hindrances. The linker is usually 3-44 amino acid residues in length. Preferably, the linker has 3-30 amino acid residues. In some embodiments, the linker has 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues.

The linker sequence may be a naturally occurring sequence or a non-naturally occurring sequence. If used for therapeutic purposes, the linker is preferably non-immunogenic in the subject to which the molecule is administered. One useful group of linker sequences are linkers derived from the hinge region of heavy chain antibodies as described in WO 96/34103 and WO 94/04678. Other examples are poly-alanine linker sequences. Further preferred examples of linker sequences are Gly/Ser linkers of different length including (Gly4Ser)₄, (Gly4Ser)₃, (Gly4Ser)₂, Gly4Ser, Gly3Ser, Gly3, Gly2ser and (Gly3Ser2)₃, in particular (Gly4Ser)₃. Preferably, the linker is selected from the group consisting of (Gly4Ser)₄, (Gly4Ser)₃, and (Gly3Ser2)₃. Even more preferably, the linker is (GGGGS)₃.

In one embodiment, the linker comprised in the molecule is selected in the group consisting of (Gly4Ser)₄, (Gly4Ser)₃, (Gly4Ser)₂, Gly4Ser, Gly3Ser, Gly3, Gly2ser and (Gly3Ser2)₃, preferably is (Gly4Ser)₃. Preferably, the linker is selected from the group consisting of (Gly4Ser)₄, (Gly4Ser)₃, and (Gly3Ser2)₃.

The moiety binding to the antibody Fc region of the targeted antibody preferably binds human fragment crystallizable region (Fc region) of the antibody at endosomal pH, more specifically early endosomal pH, but not at blood physiological pH. More specifically, the moiety binding to the antibody Fc region of the targeted antibody preferably binds human Fc region of the antibody at pH 5.5-6.5, more specifically at pH 5.8-6.2, e.g. pH 6, but not at pH 6.8-7.5 or 7.0-7.5, e.g. pH 7. Accordingly, the molecule does not bind the Fc fragment of the targeted antibody in the extracellular compartment but binds the Fc fragment in the lysosome. This feature can be tested by any method known in the art, and more particularly as detailed in the Example section. It is an important aspect because it avoids any unspecific binding to antibodies other than the targeted antibody. In the extracellular compartment, the contact between the molecule and the targeted antibody is only driven by the interaction between the antigen moiety of the molecule and the antigen-binding domain of the targeted antibody.

In another particular aspect, the moiety binding to the antibody Fc region comprises the extracellular part of the FcRn and is devoid of beta-2 microglobulin. In an additional particular aspect, the moiety binding to the antibody Fc region comprises the extracellular part of the FcRn and a fragment of beta-2 microglobulin, said fragment comprising 10-90, 20-80, 30-70 or 40-60 consecutive amino acid of SEQ ID NO: 4 or a sequence having at least 80, 85, 90, 95, 96, 97, 98 or 99% of identity with said fragment.

### Antigen Moiety

In the molecule, the moiety binding to the antibody Fc region is covalently linked to the antigen of the antibody to be depleted or a fragment of said antigen which can be bound by the antibody to be depleted.

The antigen can be a peptide, a protein, a glycoprotein or a nucleic acid.

The antigen or a fragment thereof can be covalently bound either to the beta-2 microglobulin or to the extracellular part of FcRn, or both. When the antigen is a peptide or a protein, it can be linked to the moiety binding to the antibody Fc region as a protein fusion. Alternatively, if the antigen is not a peptide or a protein, other covalent link well known in the art can be used for covalently linked the antigen to this moiety.

The antigen moiety of the molecule can be bound by the targeted antibody, especially the antigen binding domain of the targeted antibody, in the extracellular compartment, especially at blood physiological pH for instance at pH 6.8-7.5 or 7.0-7.5, e.g., pH 7. This feature can be tested by any method known in the art, and more particularly as detailed in the Example section. Optionally, the interaction between the antigen moiety of the molecule and the targeted antibody can be maintained or not in the lysosome, for instance at pH 5.5-6.5, more specifically pH 5.8-6.2, for instance pH 6.

In a first aspect, the moiety binding to the antibody Fc region is a single chain. Then, the molecule comprises a single polypeptide chain comprising the extracellular part of FcRn, the beta-2 microglobulin and the antigen or the fragment thereof.

More specifically, the molecule comprises, from the N terminus to the C terminus, the beta-2 microglobulin, the region alpha1, the region alpha2, the region alpha3 and the antigen or the fragment thereof. Optionally, a peptide linker can be used for connecting the region alpha3 of the FcRn to the antigen or a fragment thereof and/or a peptide linker can be used for connecting the beta-2 microglobulin and the region alpha1 of FcRn. Optionally, the antigen or the fragment thereof can be linked to a second antigen or the fragment thereof, optionally though a linker.

Alternatively, the molecule comprises, from the N terminus to the C terminus, the beta-2 microglobulin, the antigen or the fragment thereof and the region alpha1, the region alpha2, the region alpha3. Optionally, a peptide linker can be used for connecting the antigen or a fragment thereof to the region alpha1 of the FcRn and/or a peptide linker can be used for connecting the beta-2 microglobulin and the antigen or a fragment thereof. Optionally, the region alpha3 can be linked to a second antigen or the fragment thereof, optionally though a linker.

In a second aspect, the molecule comprises two polypeptide chains, a first polypeptide chain comprising the extracellular part of FcRn and a second polypeptide chain comprising the beta-2 microglobulin, and the antigen or the fragment thereof is covalently linked to the first polypeptide chain, the second polypeptide chain or both.

More specifically, the first polypeptide chain may comprise, from the N terminus to the C terminus, the antigen or the fragment thereof, the region alpha1, the region alpha2 and the region alpha3. Optionally, a peptide linker can be used for connecting the antigen or a fragment thereof to the region alpha1 of the FcRn. Optionally, a second antigen or the fragment thereof can be linked at the N terminal end of the antigen or the fragment thereof, optionally though a linker. Optionally, a second antigen or the fragment thereof can be linked at the C terminal end of the region alpha3, optionally though a linker.

Alternatively, the first polypeptide chain may comprise, from the N terminus to the C terminus, the region alpha1, the region alpha2, the region alpha3 and the antigen or the fragment thereof. Optionally, a peptide linker can be used for connecting the region alpha3 of the FcRn to the antigen or a fragment thereof. Optionally, a second antigen or the fragment thereof can be linked at the C terminal end of the antigen or the fragment thereof, optionally though a linker.

Alternatively or in addition, the second polypeptide chain may comprise, from the N terminus to the C terminus, the antigen or the fragment thereof and the beta-2 microglobulin; or the beta-2 microglobulin and the antigen or the fragment thereof. Optionally, a peptide linker can be used for connecting the beta-2 microglobulin and the antigen or the fragment thereof. Optionally, a second antigen or the fragment thereof can be linked at the C terminal end of the antigen or the fragment thereof, optionally though a linker.

Those different aspects can be combined for in a molecule according to the present invention.

Optionally, the molecule may include several antigens or fragments thereof. The several antigens or fragments thereof can be identical or different. In a particular aspect, the antigens are different so as to deplete different antigen specific antibodies. For instance, the molecule can comprise a first antigen and a second antigen. Accordingly, the molecule comprises a first antigen or fragment thereof that can be bound by a first antibody to be depleted, and a second antigen or fragment thereof that can be bound by a second antibody to be depleted.

In this aspect, the molecule comprises a single polypeptide chain comprising the extracellular part of FcRn, the beta-2 microglobulin, a first antigen or a fragment thereof and a second antigen or a fragment thereof. More specifically, the molecule may comprise, from the N terminus to the C terminus,
the beta-2 microglobulin, the region alpha1, the region alpha2, the region alpha3, the first antigen or the fragment thereof, and the second antigen or the fragment thereof; or
the beta-2 microglobulin, the first antigen or the fragment thereof, the region alpha1, the region alpha2, the region alpha3, and the second antigen or the fragment thereof; or
the first antigen or the fragment thereof, the second antigen or the fragment thereof, the beta-2 microglobulin, the region alpha1, the region alpha2, and the region alpha3; or
the first antigen or the fragment thereof, the beta-2 microglobulin, the region alpha1, the region alpha2, the region alpha3, and the second antigen or the fragment thereof.

Preferably, the molecule may comprise, from the N terminus to the C terminus,
the beta-2 microglobulin, the region alpha1, the region alpha2, the region alpha3, the first antigen or the fragment thereof, and the second antigen or the fragment thereof; or
the beta-2 microglobulin, the first antigen or the fragment thereof, the region alpha1, the region alpha2, the region alpha3, and the second antigen or the fragment thereof.

Alternatively, the molecule comprises two polypeptide chains, a first polypeptide chain comprising the extracellular part of FcRn and a second polypeptide chain comprising the beta-2 microglobulin, a first antigen or the fragment thereof and a second antigen or the fragment thereof being covalently linked to the first polypeptide chain or to the second polypeptide chain. For instance, the molecule may comprise, non-exhaustively,
a first polypeptide chain comprising, from the N terminus to the C terminus, the first antigen or the fragment thereof, the region alpha1, the region alpha2 and the region alpha3; and a second polypeptide chain comprising, from the N terminus to the C terminus, the second antigen or the fragment thereof and the beta-2 microglobulin; or,
a first polypeptide chain comprising, from the N terminus to the C terminus, the first antigen or the fragment thereof, the region alpha1, the region alpha2 and the region alpha3; and a second polypeptide chain comprising, from the N terminus to the C terminus, the beta-2 microglobulin and the second antigen or the fragment thereof; or,
a first polypeptide chain comprising, from the N terminus to the C terminus, the region alpha1, the region alpha2, the region alpha3 and the first antigen or the fragment thereof; and a second polypeptide chain comprising, from the N terminus to the C terminus, the second antigen or the fragment thereof and the beta-2 microglobulin; or,
a first polypeptide chain comprising, from the N terminus to the C terminus, the region alpha1, the region alpha2, the region alpha3 and the first antigen or the fragment thereof; and a second polypeptide chain comprising, from the N terminus to the C terminus, the beta-2 microglobulin and the second antigen or the fragment thereof; or,
a first polypeptide chain comprising, from the N terminus to the C terminus, the first antigen or the fragment thereof, the region alpha1, the region alpha2, the region alpha3 and the second antigen or the fragment thereof; and a second polypeptide chain comprising the beta-2 microglobulin; or,
a first polypeptide chain comprising, from the N terminus to the C terminus, the first antigen or the fragment thereof, the second antigen or the fragment thereof, the region alpha1, the region alpha2, and the region alpha3; and a second polypeptide chain comprising the beta-2 microglobulin; or,
a first polypeptide chain comprising, from the N terminus to the C terminus, the region alpha1, the region alpha2, the region alpha3, the first antigen or the fragment thereof, and the second antigen or the fragment thereof; and a second polypeptide chain comprising the beta-2 microglobulin.

Preferably, the molecule may comprise
a first polypeptide chain comprising, from the N terminus to the C terminus, the first antigen or the fragment thereof, the region alpha1, the region alpha2 and the region alpha3; and a second polypeptide chain comprising, from the N terminus to the C terminus, the beta-2 microglobulin and the second antigen or the fragment thereof; or,
a first polypeptide chain comprising, from the N terminus to the C terminus, the region alpha1, the region alpha2, the region alpha3 and the first antigen or the fragment thereof; and a second polypeptide chain comprising, from the N terminus to the C terminus, the beta-2 microglobulin and the second antigen or the fragment thereof; or,
a first polypeptide chain comprising, from the N terminus to the C terminus, the first antigen or the fragment thereof, the region alpha1, the region alpha2, the region alpha3 and the second antigen or the fragment thereof; and a second polypeptide chain comprising the beta-2 microglobulin; or,
a first polypeptide chain comprising, from the N terminus to the C terminus, the first antigen or the fragment thereof, the second antigen or the fragment thereof, the region alpha1, the region alpha2, and the region alpha3; and a second polypeptide chain comprising the beta-2 microglobulin; or,
a first polypeptide chain comprising, from the N terminus to the C terminus, the region alpha1, the region alpha2, the region alpha3, the first antigen or the fragment thereof, and the second antigen or the fragment thereof; and a second polypeptide chain comprising the beta-2 microglobulin.

The antigen or the fragment thereof can be connected to the moiety binding to the antibody Fc region through a peptide linker.

Optionally, the antigen is the antigen recognized by the antibody to be depleted. Optionally, the antigen is an antigen inducing auto-antibody. Optionally, the antigen is an antigen inducing antibodies mediating a disease, especially an autoimmune disease, an inflammatory disease or a transplant rejection. The antigen can be an auto-antigen inducing an excess of immunologic response.

Optionally, the antigen is recognized by an antibody used in diagnostic imaging.

Optionally, the antigen can be selected in the following non exhaustive Table.

| | | | Uniprot Ref for |
|---|---|---|---|
| Targeted antibody | Antigen | Associated disease/disorder | Antigen |
| Anti-Ro (SS-a) | 60 kDa SS-A/Ro | SLE (Systemic lupus | P10155 |
| | ribonucleoprotein | erythematosus), Sjögren's | |
| | | syndrome | |
| Anti-La (SS-B) | Lupus La protein | SLE (Systemic lupus | P05455 |
| | | erythematosus), Sjögren's | |
| | | syndrome | |
| Anti-Sm | small nuclear | SLE (Systemic lupus | |
| | ribonucleoproteins | erythematosus) | |
| Anti-dsDNA | Double-stranded DNA | SLE (Systemic lupus | |
| | | erythematosus) | |
| Anti-Histone | Histones | SLE (Systemic lupus | |
| | | erythematosus) | |
| Anti-Scl-70 | Topoisomérase I | Systemic sclerosis | P11387 |
| | | (Scleroderma) | |
| Anti-centromere | Centromere | Systemic sclerosis/CREST | |
| | | syndrome | |
| Anti-Jo1 | histidine-tRNA ligase | Inflammatory myopathy | |
| Anti-Smith | snRNP core proteins | SLE (Systemic lupus | |
| | | erythematosus) | |
| Anti-Smith | snRNP core proteins | primary biliary cirrhosis | |
| Anti-Sp100 | Sp100 nuclear antigen | primary biliary cirrhosis | P23497 |
| Anti-glycoprotein | nucleoporin 210 kDa | primary biliary cirrhosis | Q8TEM1 |
| 210 | | | |
| Anti-actin | actin | coeliac disease | |
| Anti-CCP | cyclic citrullinated | rheumatoid arthritis | |
| | peptide | | |
| ANCA | Myeloperoxydase (p- | Vasculitis, Wegener's | |
| | ANCA) and | granulomatosis | |
| | proteinase3 (c-ANCA) | | |
| ACA | Cardiolipin | Antiphospholipid syndrome, SLE | |
| Anti-Carp | Carbamylated protein | | |
| Rheumatoid factor | Fc portion of IgG | Rheumaoid arthritis | |
| Lupus anticoagulant | phospholipids | Antiphospholipid syndrome | |
| Anti-Collagen, type | Collagen, type IV, | Goodpasture syndrome (GPS) | Q01955 |
| IV, alpha 3 | alpha 3 | | |
| Anti-thrombin | Thrombin | SLE | P00734 |
| Anti-AChR | Nicotinic | Myasthenia gravis | |
| | acetylcholine receptor | | |
| Anti-MUSK | Muscle-specific kinase | Myasthenia gravis | |
| | (MUSK) | | |
| Anti-VGCC | Voltage-gated calcium | Lambert-Eaton myasthenic | |
| | channel(P/Q-type) | syndrome | |
| Anti-Vinculin | Vinculin | Small intestinal bacterial | |
| | | overgrowth | |
| Anti-TPO antibodies | Thyroid peroxidase | Hashimoto's thyroiditis, Graves' | |
| | | disease | |
| Anti-thyroglobulin | Thyroglobulin | Hashimoto's thyroiditis | |
| Anti-thyrotropin | TSH receptor | Graves' disease | |
| receptor | | | |
| Anti-Hu (ANNA-1) | Neuronal nuclear | paraneoplastic cerebellar | |
| | proteins | degeneration, limbic | |
| | | encephalitis, encephalomyelitis, | |
| | | subacute sensory | |
| | | neuronopathy, choreathetosis | |
| Anti-Ri (ANNA-2) | Neuronal nuclear | Opsoclonus myoclonus | |
| | proteins | syndrome | |
| Anti-Tr | Glutamate receptor | Paraneoplastic cerebellar | |
| | | syndrome | |
| Anti-amphiphysin | Amphiphysin | Stiff person syndrome, | |
| | | paraneoplastic | |
| | | cerebellar degeneration | |
| Anti-GAD | Glutamate | Stiff person syndrome, diabetes | |
| | decarboxylase | mellitus type 1 | |
| Anti-VGKC | voltage-gated | Limbic encephalitis, Isaac's | |
| | potassium | Syndrome (autoimmune | |
| | channel (VGKC) | neuromyotonia) | |
| Anti-CRMP-5 | Collapsin response | Optic neuropathy, chorea | |
| | mediator protein 5 | | |
| Anti-NMDAr | N-methyl-D-aspartate | anti-NMDA receptor | |
| | receptor(NMDA) | encephalitis | |
| NMO antibody | aquaporin-4 | neuromyelitis optica (Devic's | |
| | | syndrome) | |
| Anti-desmoglein | Dsg3 (Desmoglein 3) | Pemphigus vulgaris | |
| (anti-desmosome) | and sometimes Dsg1 | | |
| Anti-hemidesmosome | hemidesmosomes | Bullous pemphigoid | |
| Anti-laminin511 | Laminin511 | Autoimmune pancreatitis | |

For instance, the antigen can be selected from the group consisting of 60 kDa SS-A/Ro ribonucleoprotein, antigen La, a double-stranded DNA, histone, snRNP core protein, glycoprotein Ilb, glycoprotein IIIa, glycoprotein Ib, glycoprotein IX, neurofascin 155, contactin 1, Topoisomerase I, centromere, histidine-tRNA ligase, sp100 nuclear antigen, nucleoporin 210 kDa, actin, cyclic citrullinated peptide, myeloperoxidase, proteinase 3, cardiolipin, carbamylated protein, phospholipid, collagen, especially, collagen type IV alpha-3, thrombin, nicotinic acetylcholine receptor, muscle-specific kinase, voltage-gated calcium channel (P/Q-type), vinculin, thyroid peroxidase, thyroglobulin, thyrotropin receptor, neuronal nuclear protein, glutamate receptor, amphiphysin, glutamate decarboxylase, voltage-gated potassium channel, collapsin response mediator protein 5, N-methyl-D-aspartate receptor, aquaporin-4, desmoglein 3, desmoglein 1, phospholipase A2 receptor, myelin oligodendrocyte glycoprotein (MOG), myelin basic protein, proteolipid protein, myelin-associated glycoprotein, myelin-associated oligodendrocyte basic protein, transaldolase, low density lipoprotein receptor related protein 4, insulin, islet antigen 2, glutamic acid decarboxylase 65, zinc transporter 8, cartilage gp39, gp130-RAPS, 65 kDa heat shock protein, fibrillarin, small nuclear protein (snoRNP), thyroid stimulating factor receptor, nuclear antigens, glycoprotein gp70, ribosomes, pyruvate dehydrogenase dehydrolioamide acetyltransferase, hair follicle antigens, human tropomyosin isoform 5, a-amino-3-hydroxy-5-methyl-4-isoxazolepropionic acid (AMP A) receptor, GABAA and GABAB receptors, glycine receptor, and dipeptidyl-peptidase-like protein 6 (DPPX).

More specifically, the antigen can be selected from the group consisting of 60 kDa SS-A/Ro ribonucleoprotein, antigen La, a double-stranded DNA, histone, snRNP core protein, glycoprotein IIb, glycoprotein Illa, glycoprotein Ib, glycoprotein IX, neurofascin 155, contactin 1, Topoisomerase I, centromere, histidine-tRNA ligase, sp100 nuclear antigen, nucleoporin 210 kDa, actin, cyclic citrullinated peptide, myeloperoxidase, proteinase 3, cardiolipin, carbamylated protein, phospholipid, collagen type IV alpha-3, thrombin, nicotinic acetylcholine receptor, muscle-specific kinase, voltage-gated calcium channel (P/Q-type), vinculin, thyroid peroxidase, thyroglobulin, thyrotropin receptor, neuronal nuclear protein, glutamate receptor, amphiphysin, glutamate decarboxylase, voltage-gated potassium channel, collapsin response mediator protein 5, N-methyl-D-aspartate receptor, aquaporin-4, desmoglein 3, desmoglein 1, and phospholipase A2 receptor.

More specifically, the antigen can be selected from the group consisting of nicotinic acetylcholine receptor, muscle-specific kinase, desmoglein 3, desmoglein 1, glycoprotein IIb, glycoprotein Illa, glycoprotein Ib, glycoprotein IX, thyrotropin receptor, thyroid peroxidase, snRNP core protein, histone, antigen La and 60 kDa SS-A/Ro ribonucleoprotein.

In a very specific aspect, the antigen can be selected from desmoglein 3 (DSG3), desmoglein 1 (DSG1) and the combination thereof. These antigens are specific of auto-antibodies mediating pemphigus vulgaris. In another very specific aspect, the antigen can be selected from nicotinic acetylcholine receptor (Achr), muscle-specific kinase (MusK), and the combination thereof. These antigens are specific of auto-antibodies mediating myasthenia gravis.

In an additional very specific aspect, the antigen can be selected from glycoprotein IIb (GpIIb), glycoprotein IIIa (GpIIIa), glycoprotein Ib (GpIb), glycoprotein IX and any combination thereof. These antigens are specific of auto-antibodies mediating idiopathic thromobocytopenic purpura (ITP).

In another additional very specific aspect, the antigen can be the extracellular domain of myelin oligodendrocyte glycoprotein (MOG) and it can be useful for the treatment of multiple sclerosis.

### Production and Nucleic Acid, Vector and Host Cells

To produce the molecule according to the present invention by mammalian cells, nucleic acid sequences or group of nucleic acid sequences coding for the molecule of the present invention are subcloned into one or more expression vectors. Such vectors are generally used to transfect mammalian cells.

Generally, such method comprises the following steps of:
(1) transfecting or transforming appropriate host cells with the polynucleotide(s) encoding the molecule of the invention or the vector containing the polynucleotide(s);
(2) culturing the host cells in an appropriate medium; and
(3) optionally isolating or purifying the molecule from the medium or host cells.

The invention further relates to a nucleic acid or a set of nucleic acids encoding the molecule as disclosed above, a vector, preferably an expression vector, comprising the nucleic acid of the invention, a genetically engineered host cell transformed with the vector of the invention or directly with the nucleic acid or set of nucleic acids encoding the molecule, and a method for producing the protein of the invention by recombinant techniques.

The nucleic acid, the vector and the host cells are more particularly described hereafter.

The invention also relates to a nucleic acid molecule or a set of nucleic acid molecules encoding the molecule as defined above, wherein the molecule comprises
an extracellular part of a human neonatal Fc receptor (FcRn) including regions alpha1, alpha2 and alpha3 and devoid of transmembrane domain and
a beta-2 microglobulin;
said extracellular part of FcRn and/or said beta-2 microglobulin being covalently linked to the antigen of the antibody to be depleted or a fragment of said antigen which can be bound by the antibody to be depleted.

Nucleic acids encoding the molecule disclosed herein can be amplified by any techniques known in the art, such as PCR. Such nucleic acid may be readily isolated and sequenced using conventional procedures.

In a first aspect, the nucleic acid molecule(s) encoding the molecule as defined herein comprises:
a first nucleic acid molecule encoding the extracellular part of the FcRn, and optionally one or several antigens or fragments thereof, and
a second nucleic acid molecule encoding the beta-2 microglobulin, and optionally one or several antigens or fragments thereof.

In a second aspect, the nucleic acid molecule(s) encoding the molecule as defined herein comprises a nucleic acid molecule encoding the extracellular part of the FcRn, the beta-2 microglobulin, and one or several antigens or fragments thereof.

In one embodiment, the nucleic acid molecule is an isolated, particularly non-natural, nucleic acid molecule.

In another aspect, the invention relates to a vector comprising the nucleic acid molecule or the group of nucleic acid molecules as defined above.

As used herein, a “vector” is a nucleic acid molecule used as a vehicle to transfer genetic material into a cell. The term “vector” encompasses plasmids, viruses, cosmids and artificial chromosomes. In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. Modern vectors may encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The nucleic acid molecule encoding the molecule can be cloned into a vector by those skilled in the art, and then transformed into host cells. These methods include in vitro recombinant DNA techniques, DNA synthesis techniques, in vivo recombinant techniques, etc. The methods known to the artisans in the art can be used to construct an expression vector containing the nucleic acid sequence encoding the molecule and appropriate regulatory components for transcription/translation.

Accordingly, the present invention also provides a recombinant vector, which comprises a nucleic acid molecule or a set of nucleic acid molecules encoding the molecule according to the present invention. In one preferred embodiment, the expression vector further comprises a promoter and a nucleic acid sequence encoding a secretion signal peptide, and optionally at least one drug-resistance gene for screening. The expression vector may further comprise a ribosome-binding site for initiating the translation, transcription terminator and the like.

Suitable expression vectors typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence.

An expression vector can be introduced into host cells using a variety of techniques including calcium phosphate transfection, liposome-mediated transfection, electroporation, and the like. Preferably, transfected cells are selected and propagated wherein the expression vector is stably integrated in the host cell genome to produce stable transformants.

In another aspect, the invention relates to a host cell comprising a vector or a nucleic acid molecule or group of nucleic acid molecules as defined above, for example for molecule production purposes.

As used herein, the term “host cell” is intended to include any individual cell or cell culture that can be or has been recipient of vectors, exogenous nucleic acid molecules, and polynucleotides encoding the molecule according to the present invention. The term “host cell” is also intended to include progeny or potential progeny of a single cell. Suitable host cells include prokaryotic or eukaryotic cells, and also include but are not limited to bacteria, yeast cells, fungi cells, plant cells, and animal cells such as insect cells and mammalian cells, e.g., murine, rat, rabbit, macaque or human.

Suitable hosts cells are especially eukaryotic hosts cells which provide suitable post-translational modifications such as glycosylation. Preferably, such suitable eukaryotic host cell may be fungi such as Pichia pastoris, Saccharomyces cerevisiae, Schizosaccharomyces pombe; insect cell such as Mythimna separate; plant cell such as tobacco, and mammalian cells such as BHK cells, 293 cells, CHO cells, NSO cells and COS cells.

Preferably, the host cell of the present invention is selected from the group consisting of CHO cell, COS cell, NSO cell, and HEK cell.

Then host cells stably or transiently express the molecule according to the present invention. Such expression methods are known by the man skilled in the art.

A method of production of the molecule is also provided herein. The method comprises culturing a host cell comprising a nucleic acid encoding the molecule as provided above, under conditions suitable for its expression, and optionally recovering the molecule from the host cell (or host cell culture medium).

Particularly, for recombinant production of a molecule, nucleic acid encoding a molecule, e.g., as described above, is isolated and inserted into one or more vectors for further cloning and/or expression in a host cell. The molecules are then isolated and/or purified by any methods known in the art. These methods include, but are not limited to, conventional renaturation treatment, treatment by protein precipitant (such as salt precipitation), centrifugation, cell lysis by osmosis, sonication, supercentrifugation, molecular sieve chromatography or gel chromatography, adsorption chromatography, ion exchange chromatography, HPLC, any other liquid chromatography, and the combination thereof. As described, for example, by Coligan, molecule isolation techniques may particularly include affinity chromatography, size-exclusion chromatography and ion exchange chromatography.

### Diseases

The molecules according to the present invention can have a broad utility. For instance, they can be used for the clearance of deleterious antibodies for therapy but also diagnosis. Indeed, they could be used for the treatment of antibody-mediated autoimmunity, antibody-mediated inflammatory disease, antibody-mediated transplant rejection and the clearance of background during diagnostic imaging.

Accordingly, the present invention relates to a pharmaceutical composition comprising a molecule as described herein, wherein the molecule comprises
an extracellular part of a human neonatal Fc receptor (FcRn) including regions alpha1, alpha2 and alpha3 and devoid of transmembrane domain and
a beta-2 microglobulin;
said extracellular part of FcRn and/or said beta-2 microglobulin being covalently linked to the antigen of the antibody to be depleted or a fragment of said antigen which can be bound by the antibody to be depleted.

Optionally, the pharmaceutical composition further comprise a pharmaceutically acceptable carrier, excipient, or salt.

The present invention also relates to a pharmaceutical composition comprising a molecule described herein, the nucleic acid molecule, the group of nucleic acid molecules, the vector and/or the host cells as described hereabove, preferably as the active ingredient or compound. The formulations can be sterilized and, if desired, mixed with auxiliary agents such as pharmaceutically acceptable carriers, excipients, salts, anti-oxidant and/or stabilizers which do not deleteriously interact with the molecule of the invention, nucleic acid, vector and/or host cell of the invention and does not impart any undesired toxicological effects. Optionally, the pharmaceutical composition may further comprise an additional therapeutic agent.

Particularly, the pharmaceutical composition according to the invention can be formulated for any conventional route of administration including a topical, enteral, oral, parenteral, intranasal, intravenous, intramuscular, subcutaneous or intraocular administration and the like. To facilitate administration, the molecule as described herein can be made into a pharmaceutical composition for in vivo administration. The means of making such a composition have been described in the art (see, for instance, Remington: The Science and Practice of Pharmacy, Lippincott Williams & Wilkins, 21st edition (2005).

The pharmaceutical composition may be prepared by mixing a molecule having the desired degree of purity with optional pharmaceutically acceptable carriers, excipients, anti-oxidant, and/or stabilizers in the form of lyophilized formulations or aqueous solutions. Such suitable carriers, excipients, anti-oxidant, and/or stabilizers are well known in the art and have been for example described in Remington's Pharmaceutical Sciences 16th edition, Osol, A. Ed. (1980).

To facilitate delivery, any of the molecule or its encoding nucleic acids can be conjugated with a chaperon agent. The chaperon agent can be a naturally occurring substance, such as a protein (e.g., human serum albumin, low-density lipoprotein, or globulin), carbohydrate (e.g., a dextran, pullulan, chitin, chitosan, inulin, cyclodextrin or hyaluronic acid), or lipid. It can also be a recombinant or synthetic molecule, such as a synthetic polymer, e.g., a synthetic polypeptide.

Pharmaceutical compositions according to the invention may be formulated to release the active ingredients (e.g. the molecule of the invention) substantially immediately upon administration or at any predetermined time or time period after administration. The pharmaceutical composition in some aspects can employ time-released, delayed release, and sustained release delivery systems such that the delivery of the composition occurs prior to, and with sufficient time to cause, sensitization of the site to be treated. Means known in the art can be used to prevent or minimize release and absorption of the composition until it reaches the target tissue or organ, or to ensure timed-release of the composition. Such systems can avoid repeated administrations of the composition, thereby increasing convenience to the subject and the physician.

It will be understood by one skilled in the art that the formulations of the invention may be isotonic with human blood that is the formulations of the invention have essentially the same osmotic pressure as human blood. Such isotonic formulations generally have an osmotic pressure from about 250 mOSm to about 350 mOSm. Isotonicity can be measured by, for example, a vapor pressure or ice-freezing type osmometer.

Pharmaceutical composition typically must be sterile and stable under the conditions of manufacture and storage. Prevention of presence of microorganisms may be ensured both by sterilization procedures (for example by microfiltration), and/or by the inclusion of various antibacterial and antifungal agents

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect.

The present invention relates the molecule as described herein or the pharmaceutical composition comprising it for use as a drug, wherein the molecule comprises
an extracellular part of a human neonatal Fc receptor (FcRn) including regions alpha1, alpha2 and alpha3 and devoid of transmembrane domain and
a beta-2 microglobulin;
said extracellular part of FcRn and/or said beta-2 microglobulin being covalently linked to the antigen of the antibody to be depleted or a fragment of said antigen which can be bound by the antibody to be depleted.

Indeed, the molecule can be adapted for the treatment of any disease or disorder mediated by an antibody or an excessive amount of an antibody directed against one particular antigen or group of antigens, since the molecule has the capacity of selective depletion or clearance of the targeted antibody.

Thus, the present invention relates to:
the molecule as described herein or the pharmaceutical composition comprising it for use for the treatment of a disease or disorder mediated by an antibody; or
the use of the molecule as described herein or the pharmaceutical composition comprising it for the manufacture of a medicine for the treatment of a disease or disorder mediated by an antibody; or
a method for treating a disease or disorder mediated by an antibody in a subject, comprising administering a therapeutically effective amount of the molecule as described herein or the pharmaceutical composition comprising it to the subject;
wherein the molecule comprises
   an extracellular part of a human neonatal Fc receptor (FcRn) including regions alpha1, alpha2 and alpha3 and devoid of transmembrane domain and
   a beta-2 microglobulin;
said extracellular part of FcRn and/or said beta-2 microglobulin being covalently linked to the antigen of the antibody to be depleted or a fragment of said antigen which can be bound by the antibody to be depleted.

The disease or disorder mediated by an antibody can be an autoimmune disease or disorder, an inflammatory disease or disorder, or a transplant rejection.

Optionally, the disease is an autoimmune disease and the targeted antibody specifically binds an autoantigen and the molecule comprises an antigen moiety comprising the autoantigen or a fragment thereof which can be bound by the targeted antibody.

Optionally, the disease is a transplant rejection of a transplanted organ, the targeted antibody specifically binds to an antigen on the transplanted organ, and the molecule comprises an antigen moiety comprising the antigen on the transplanted organ or a fragment thereof which can be bound by the targeted antibody.

Optionally, the disease to be treated is selected in the group consisting of Myasthenia Gravis, Pemphigus vulgaris, systemic lupus erythematosus, Sjögren's syndrome, immune thrombocytopenia (especially persistent or chronic immune thrombocytopenia), chronic inflammatory demyelinating polyneuropathy, scleroderma, CREST syndrome, inflammatory myopathy, primary biliary cirrhosis, coeliac disease, rheumatoid arthritis, granulomatosis, antiphospholipid syndrome, Goodpasture syndrome, chronic autoimmune hepatitis, polymyositis, small intestinal bacterial overgrowth, Hashimoto's thyroiditis, Graves' disease, paraneoplastic cerebellar degeneration, limbic encephalitis, encephalomyelitis, subacute sensory neuronopathy, choreoathetosis, opsoclonus myoclonus syndrome, Stiff-Person syndrome, diabetes mellitus type 1, Isaac's syndrome, optic neuropathy, anti-N-Methyl-D-Aspartate Receptor Encephalitis, neuromyelitis optica, Bullous pemphigoid, membranous nephropathy, allogenic islet graft rejection, alopecia areata, ankylosing spondylitis, autoimmune Addison's disease, Alzheimer's disease, antineutrophil cytoplasmic autoantibodies (ANCA), autoimmune diseases of the adrenal gland, autoimmune hemolytic anemia, autoimmune myocarditis, autoimmune neutropenia, autoimmune oophoritis and orchitis, autoimmune urticaria, Behcet's disease, cardiomyopathy, Castleman's syndrome, celiac spruce-dermatitis, chronic fatigue immune disfunction syndrome, Churg-Strauss syndrome, cicatrical pemphigoid, CREST syndrome, cold agglutinin disease, Crohn's disease, dermatomyositis, discoid lupus, epidermolysis bullosa acquisita, essential mixed cryoglobulinemia, fibromyalgia-fibromyositis, glomerulonephritis, Guillain-Barre syndrome, graft-versus-host disease (GVHD), hemophilia A, idiopathic membranous neuropathy, idiopathic pulmonary fibrosis, idiopathic thrombocytopenia purpura (ITP), IgA neuropathy, IgM polyneuropathies, juvenile arthritis, Kawasaki's disease, lichen plantus, lichen sclerosus, Meniere's disease, mixed connective tissue disease, mucous membrane pemphigoid, multiple sclerosis, type 1 diabetes mellitus, Multifocal motor neuropathy (MMN), pemphigoid gestationis, pemphigus foliaceus, pernicious anemia, polyarteritis nodosa, polychrondritis, polyglandular syndromes, polymyalgia rheumatica, primary agammaglobulinemia, psoriasis, psoriatic arthritis, relapsing polychondritis, Reynauld's phenomenon, Reiter's syndrome, sarcoidosis, solid organ transplant rejection, Takayasu arteritis, toxic epidermal necrolysis (TEN), Stevens Johnson syndrome (SJS), temporal arteristis/giant cell arteritis, thrombotic thrombocytopenia purpura, ulcerative colitis, uveitis, dermatitis herpetiformis vasculitis, anti-neutrophil cytoplasmic antibody-associated vasculitides, vitiligo, asthma, autoimmune pancreatitis, IgA nephropathy and Wegner's granulomatosis; optionally selected in the group consisting of Myasthenia Gravis, Pemphigus vulgaris, systemic lupus erythematosus, Sjögren's syndrome, immune thrombocytopenia (especially persistent or chronic immune thrombocytopenia), chronic inflammatory demyelinating polyneuropathy, scleroderma, CREST syndrome, inflammatory myopathy, primary biliary cirrhosis, coeliac disease, rheumatoid arthritis, granulomatosis, antiphospholipid syndrome, Goodpasture syndrome, chronic autoimmune hepatitis, polymyositis, small intestinal bacterial overgrowth, Hashimoto's thyroiditis, Graves' disease, paraneoplastic cerebellar degeneration, limbic encephalitis, encephalomyelitis, subacute sensory neuronopathy, choreoathetosis, opsoclonus myoclonus syndrome, Stiff-Person syndrome, diabetes mellitus type 1, Isaac's syndrome, optic neuropathy, anti-N-Methyl-D-Aspartate Receptor Encephalitis, neuromyelitis optica, Bullous pemphigoid, and membranous nephropathy, preferably selected in the group consisting of Myasthenia Gravis, Pemphigus vulgaris, systemic lupus erythematosus, Sjögren's syndrome, antiphospholipid syndrome, Hashimoto's thyroiditis and Graves' disease.

Thus, the present invention relates to:
the molecule as described herein or the pharmaceutical composition comprising it for use for depleting an antibody specific the antigen, especially for the treatment of a disease or disorder mediated by the antibody specific the antigen; or
the use of the molecule as described herein or the pharmaceutical composition comprising it for the manufacture of a medicine for depleting an antibody specific the antigen, especially for the treatment of a disease or disorder mediated by the antibody specific the antigen; or
a method for depleting an antibody specific of an antigen in a subject, comprising administering a therapeutically effective amount of the molecule as described herein n or the pharmaceutical composition comprising it to the subject,
wherein the molecule comprises
   an extracellular part of a human neonatal Fc receptor (FcRn) including regions alpha1, alpha2 and alpha3 and devoid of transmembrane domain and
   a beta-2 microglobulin;
said extracellular part of FcRn and/or said beta-2 microglobulin being covalently linked to the antigen of the antibody to be depleted or a fragment of said antigen which can be bound by the antibody to be depleted.

The molecule is administered in an amount sufficient to remove at least 10, 20, 30, 40, 50, 60, 70, 80 or 90% of the antibody specific of the antigen from blood circulation or a target tissue of the patient. Optionally, the molecule is administered in an amount sufficient to remove at least 10, 20, 30, 40, 50, 60, 70, 80 or 90% of the antibody specific of the antigen from blood circulation or a target tissue of the patient within 1, 2, 3, 4, or 5 days of the administration.

Optionally, the molecule removes less than 10, 5, 4, 3, 2 or 1% of the non-targeted antibodies in the blood circulation or a target tissue of the patient. Optionally, the molecule removes an amount of non-targeted antibodies in the blood circulation or a target tissue of the patient that does not cause a clinically adverse effect in the patient.

Optionally, the molecule if for use in imaging targeting an antigen, the molecule allowing to increase contrast during imaging by depleting the antibodies specific of the antigen and the molecule comprises an antigen moiety including the antigen or a fragment thereof which can be specifically bound by the antibodies specific of the antigen.

### EXAMPLES

### Results

### Example 1: Pharmacokinetics of FcRn Molecules in Mice

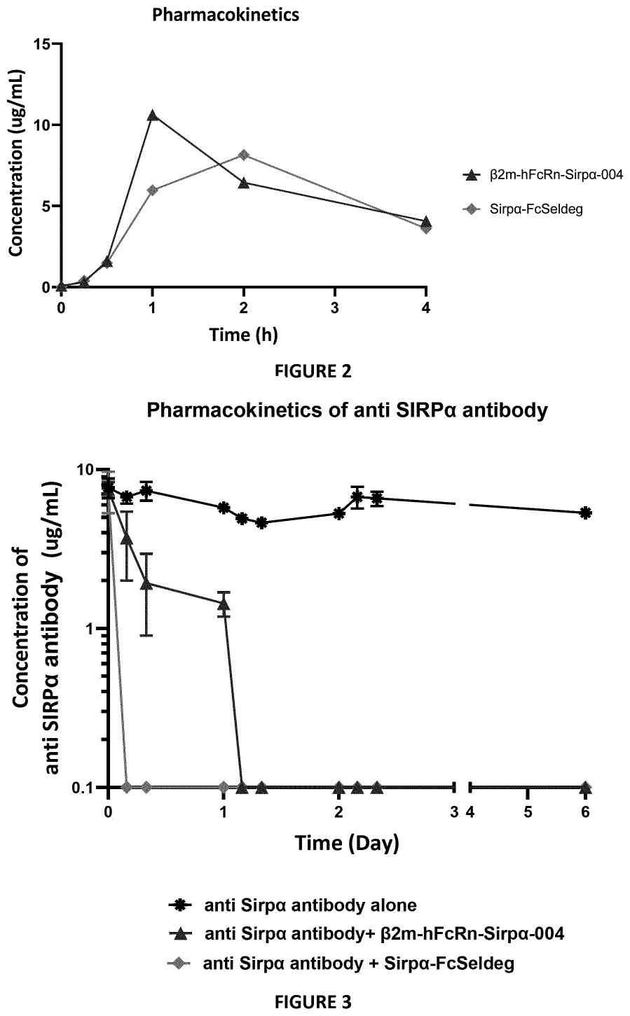

Pharmacokinetics study of the β2m-hFcRn-SIRPα-004 and SIRPα-FcSeldeg such as described in FIG. 2 was assessed.

Immunocompetent 6 weeks old Balb/c mice were intraperitoneally injected with one dose of FcRn molecule (100 ag/injection). Concentration of the FcRn molecules in the sera was assessed by ELISA at multiple time points following injection using an anti-SIRPα antibody immobilized, then serum-containing drugs were added. Detection was performed with biotinylated mouse anti-HIS (MBL #D291-6) and peroxidase-labeled streptavidin (Jackson immunoresearch; USA; reference 016-030-084) were added and revealed by conventional methods.

Results: Pharmacokinetics were compared for all FcRn molecules described in the FIG. 2. Profiles of the β2m-hFcRn-SIRPα-004 and SIRPα-FcSeldeg were shown after a single intraperitoneal injection. A good pharmacokinetics profile was observed.

### Example 2: Pharmacokinetics of Anti-SIRPα Antibody in Mice in Presence of FcRn Molecules

Pharmacokinetics study of the anti SIRPα antibody as shown in FIG. 3 was assessed in presence of FcRn molecules or SIRPα-FcSeldeg.

Immunocompetent 6 weeks old Balb/c mice were intraperitoneally injected with one dose of anti SIRPα antibody at day −1 (25 ag/injection) and several doses of FcRn molecules at day 0, day 0+4h, day 0+8h, day 1, day 1+4h, day 1+8h, day 2, day 2+4h, day 2+8h (100 ag/injection). Concentration of the anti-SIRPα antibody in the sera was assessed by ELISA at multiple time points following injection using mouse anti-human kappa antibody immobilized, then serum-containing antibodies and drugs were added. Detection was performed with donkey anti-human HRP (#709-035-149, Jackson Laboratory) and revealed by conventional methods.

Results: Pharmacokinetics profile of the anti SIRPα antibody in presence of FcRn molecules or SIRPα-FcSeldeg was shown in FIG. 3.

A decrease of anti SIRPα antibody concentration was observed after intraperitoneal injection of β2m-hFcRn-SIRPα-004. This decrease kinetics was observed from the first injection and the total elimination kinetics was observed from the fourth injection at day 2 compared to anti SIRPα antibody alone.

The total elimination kinetics was observed from the first injection compared to anti SIRPα antibody alone after intraperitoneal injection of SIRPα-FcSeldeg.

### Example 3: Pharmacokinetics of Anti SIRPα Antibody (A) and Anti IL7Rα Antibody (B) in Mice

Pharmacokinetics study of the anti SIRPα antibody and anti-IL7Rα antibody as shown in FIG. 4 was assessed in presence of FcRn molecules or SIRPα-FcSeldeg.

Immunocompetent 6 weeks old Balb/c mice were intraperitoneally co-injected with one dose of anti SIRPα antibody and anti IL7Rα antibody at day 0 (25 ag/injection) and several doses of FcRn molecules at day 1, day1+4h, day1+8h, day 2, day2+4h, day2+8h (100 ag/injection). Concentration of the anti SIRPα antibody in the sera was assessed by ELISA, at multiple time points following injection, using anti-idiotype-SIRPα antibody immobilized, then serum-containing antibodies and drugs were added. Detection was performed with donkey anti-human HRP (#709-035-149, Jackson Laboratory) and revealed by conventional methods.

Concentration of the anti-IL7Rα antibody in the sera was assessed by ELISA, at multiple time points following injection, using ELISA using anti-idiotype IL7Rα antibody immobilized, then serum-containing antibodies and drugs were added. Detection was performed with donkey anti-human HRP (#709-035-149, Jackson Laboratory) and revealed by conventional methods.

Results: Pharmacokinetics profile of the anti-SIRPα antibody and anti-IL7Rα antibody in presence of FcRn molecules or SIRPα-FcSeldeg were shown in FIG. 4.

A decrease of the anti SIRPα antibody concentration was observed after intraperitoneal injection of β2m-hFcRn-SIRPα-004. This decrease kinetics was observed from the first injection and the total elimination kinetics was observed at day 7 compared to anti SIRPα antibody alone.

The total elimination kinetics was observed from the first injection compared to anti SIRPα antibody alone after intraperitoneal injection of SIRPα-FcSeldeg. However, a decrease of anti IL7Rα antibody after intraperitoneal injection of SIRPα-FcSeldeg was further observed at day 7 compared to control group injected with anti SIRPα antibody and anti IL7Rα antibody alone. Therefore, the effect of SIRPα-FcSeldeg is not specific of the anti SIRPα antibody and SIRPα-FcSeldeg also shows an effect on the concentration of a non-relevant antibody such as anti IL7Rα antibody.

On the contrary, no significant modification of the anti IL7Rα antibody concentration was observed after intraperitoneal injection of β2m-hFcRn-SIRPα-004. Thus, β2m-hFcRn-SIRPα-004 molecules present a specificity regarding the antibody and have only an effect on the targeted antibody, namely the anti SIRPα antibody.

### Example 4: Pharmacokinetics of Anti SIRPα Antibody in Mice in Presence of Ascending Doses of β2m-hFcRn-SIRPα-004

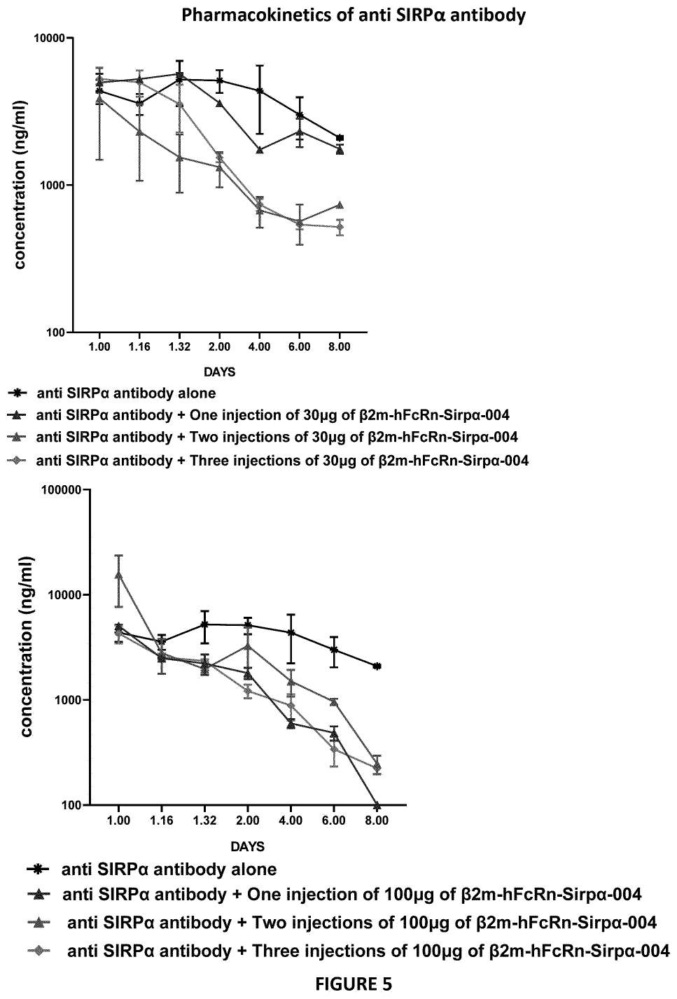

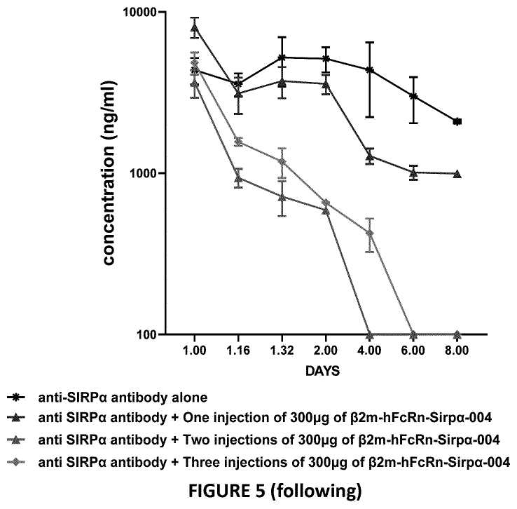

Pharmacokinetics study of the anti SIRPα antibody as shown in FIG. 5 was assessed in presence of β2m-hFcRn-SIRPα-004.

Immunocompetent 6 weeks old Balb/c mice were intraperitoneally injected with one doses of anti SIRPα antibody at day 0 (25 ag/injection) and one dose of β2m-hFcRn-SIRPα-004 at day 1 or two doses at day 1 and day 1+4h or three doses at day 1 and day 1+4 h and day 1+8h. Concentration of the anti SIRPα antibody in the sera was assessed by ELISA at multiple time points following injection using mouse anti-human kappa antibody immobilized, then serum-containing antibodies and drugs were added. Detection was performed with donkey anti-human HRP (#709-035-149, Jackson Laboratory) and revealed by conventional methods.

### Results:

First graph: Pharmacokinetics study of the anti-SIRPα antibody was assessed in presence of one, two or three doses of 30 μg of β2m-hFcRn-SIRPα-004. A decrease of the anti SIRPα antibody concentration was observed after two or three intraperitoneal injections of β2m-hFcRn-SIRPα-004 compared to control group. No significant modification of anti SIRPα antibody was observed with one dose at 30 μg.

Second graph: Pharmacokinetics study of the anti SIRPα antibody was assessed in presence of one, two or three doses of 100 μg of β2m-hFcRn-SIRPα-004. A decrease of the anti SIRPα antibody concentration was observed after one, two or three intraperitoneal injections of β2m-hFcRn-SIRPα-004 compared to control group.

Third graph: Pharmacokinetics study of the anti SIRPα antibody was assessed in presence of one, two or three doses of 300 μg of β2m-hFcRn-SIRPα-004. A decrease of the anti SIRPα antibody concentration was observed after one, two or three intraperitoneal injections of 300 μg of β2m-hFcRn-SIRPα-004 compared to control group. Nevertheless, a partial decrease was observed after one injection at 300 μg with β2m-hFcRn-SIRPα-004 at day 8. In contrast, with two or three injections at 300 μg, a complete decrease was observed at day 4 or at day 6, respectively.

### Example 5: Pharmacokinetics of Anti-SIRPα Antibody in Mice in Presence of FcRn Molecules

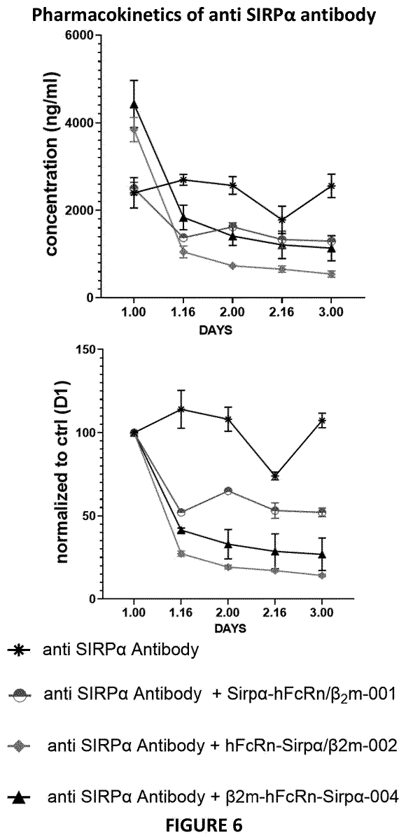

Pharmacokinetics study of the anti SIRPα antibody as shown in FIG. 6 was assessed in presence of FcRn molecules.

Immunocompetent 6 weeks old Balb/c mice were intraperitoneally injected with one dose of anti SIRPα antibody at day 0 (25 μg) and two doses of FcRn molecules at day 1 and day 1+4h (300 μg). Concentration of the anti SIRPα antibody in the sera was assessed by ELISA, at multiple time points following injection, using mouse anti-human kappa antibody immobilized, then serum-containing antibodies and drugs were added. Detection was performed with donkey anti-human HRP (#709-035-149, Jackson Laboratory) and revealed by conventional methods.

Results: Pharmacokinetics profile of the anti-SIRPα antibody in presence of FcRn molecules composed by two chains (dimeric forms) was shown in FIG. 6. A decrease of the anti SIRPα antibody concentration from the first injection was observed with β2m-hFcRn-SIRPα-004 and hFcRn-Sirpa/β2m-002 compared to anti SIRPα antibody alone. The 75% elimination kinetics of anti SIRPα antibody at day 2 was observed with β2m-hFcRn-SIRPα-004 and hFcRn-Sirpa/β2m-002 compared to anti SIRPα antibody alone. A decrease of the anti SIRPα antibody concentration from the first injection around 50% compared to anti SIRPα antibody alone was observed with Sirpa-hFcRn/P₂m-001.

The decrease of the anti SIRPα antibody concentration with hFcRn-Sirpa/β2m-002 was better than with Sirpa-hFcRn/P₂m-001. The decrease of the anti SIRPα antibody concentration with hFcRn-Sirpa/β2m-002 was equal to the decrease observed with β2m-hFcRn-SIRPα-004.

### Example 6: Pharmacokinetics of Anti-SIRPα Antibody and Anti IL-7Rα Antibody in Mice in Presence of FcRn Molecules

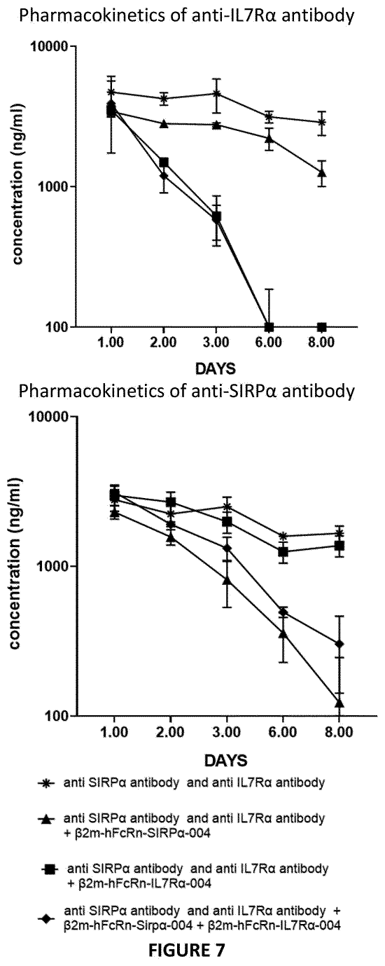

Pharmacokinetics study of the anti SIRPα antibody as shown in FIG. 7 was assessed in presence of FcRn molecules to validate a bispecific model.

Immunocompetent 7 weeks old Balb/c mice were intraperitoneally co-injected with one dose of anti-SIRPα antibody and anti IL-7Rα antibody at day 0 (25 μg) and two doses of FcRn molecules at day 1 and day 2 (100 μg). Concentration of the anti-SIRPα antibody in the sera was assessed by ELISA, at multiple time points following injection, using anti-idiotype SIRPα antibody immobilized, then serum-containing antibodies and drugs were added. Detection was performed with donkey anti-human HRP (#709-035-149, Jackson Laboratory) and revealed by conventional methods.

Concentration of the anti IL-7Rα antibody in the sera was assessed by ELISA, at multiple time points following injection, using anti-idiotype IL7Rα antibody immobilized, then serum-containing antibodies and drugs were added. Detection was performed with donkey anti-human HRP (#709-035-149, Jackson Laboratory) and revealed by conventional methods.

Results: Pharmacokinetics profile of the anti SIRPα antibody and anti IL-7Rα antibody in presence of FcRn molecules were shown.

A total decrease of anti SIRPα antibody concentration at day 8 (B) and no modification of anti IL-7Rα antibody concentration (A) were observed with injection of β2m-hFcRn-Sirpa-004.

A total decrease of anti IL-7Rα antibody concentration at day 6 (A) and no modification of anti SIRPα antibody concentration (B) were observed with injection of β2m-hFcRn-IL7Rα-004.

A total decrease of anti IL-7Rα antibody concentration at day 6 (A) and anti SIRPα antibody concentration at day 8 (B) were observed with co injection of β2m-hFcRn-Sirpa-004 and β2m-hFcRn-IL7Rα-004.

Thus, a bispecific model was assessed and validated to decrease two specific antibodies instead of one using two FcRn molecules instead of one.

### Example 7: Pharmacokinetics of Anti SIRPα Antibody and Anti IL-7Rα Antibody in Mice in Presence of FcRn Mutated Molecules

Pharmacokinetics study of the anti SIRPα antibody as shown in FIG. 8 was assessed in presence of FcRn molecules, FcRn mutated molecules and ARGX113.

Immunocompetent 7 weeks old Balb/c mice were intraperitoneally co-injected with one dose of anti SIRPα antibody and anti IL-7Rα antibody at day 0 (25 μg) and one dose of FcRn molecules at day 1 (100 μg or 300 μg). Concentration of the anti SIRPα antibody in the sera was assessed by ELISA, at multiple time points following injection, using anti-idiotype SIRPα antibody immobilized, then serum-containing antibodies and drugs were added. Detection was performed with donkey anti-human HRP (#709-035-149, Jackson Laboratory) and revealed by conventional methods.

Concentration of the anti IL-7Rα antibody in the sera was assessed by ELISA at multiple time points following injection using CD127-Fc (CD127-fc; 306-IR) immobilized, then serum-containing antibodies and drugs were added. Detection was performed with mouse anti-human kappa antibody (#1.02 mg:ml, 18/04/18). After incubation and washing, donkey Anti-MsPO (JI #715-036-151, lot 104986) was added and revealed by conventional methods

Results: Pharmacokinetics profile of the anti SIRPα antibody and anti IL-7Rα antibody in presence of FcRn molecules, FcRn mutated molecules and ARGX113 were shown.

A total decrease of anti IL-7Rα antibody concentration at day 2 (B) and a total decrease of anti SIRPα antibody concentration at day 7 (A) were observed with injection of ARGX113.

No significant modification of anti IL-7Rα antibody concentration (B) and no significant modification of anti SIRPα antibody concentration (A) were observed with injection of β2m-hFcRn.

A total decrease of anti IL-7Rα antibody concentration at day 2 (B) and no modification of anti SIRPα antibody concentration (A) were observed with injection of β2m-hFcRn-IL7Rα-004.

A total decrease of anti SIRPα antibody concentration at day 7 (A) and no modification of anti IL7Rα antibody concentration (B) compared to control group were observed with injection of β2m-hFcRn-SIRPα-004.

The same decrease of anti SIRPα antibody concentration at day 7 (A) and no modification of anti IL7Rα antibody concentration (B) compared to β2m-hFcRn-SIRPα-004 were observed with all mutated FcRn molecules: β2m-hFcRn-H166A-Sirpa-010 (100 μg), β2m-hFcRn-W51A-Sirpa-011 (300 μg), β2m-hFcRn-W53A-Sirpa-012 (300 μg), β2m-hFcRn-W59A-Sirpa-013 (300 μg), β2m-hFcRn-W61A-Sirpa-014 (300 μg), and β2m-hFcRn-IL7Rα-004 (300 μg).

### Example 8: Kinetics of Albumin Concentration in Mice

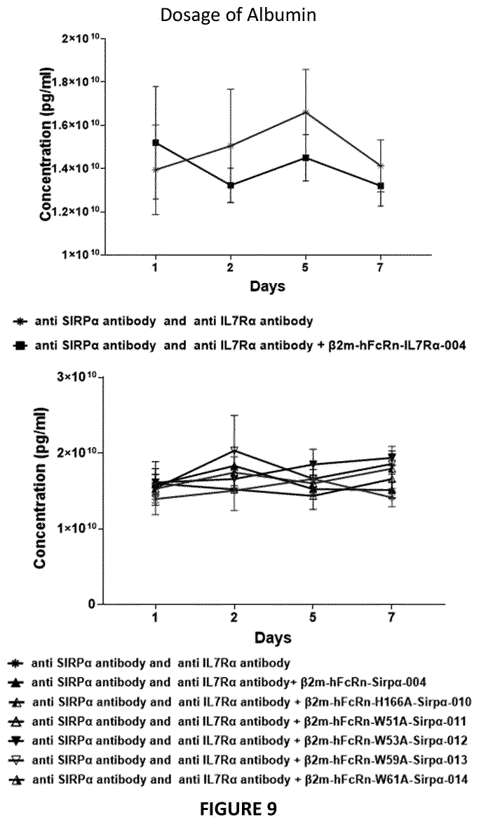

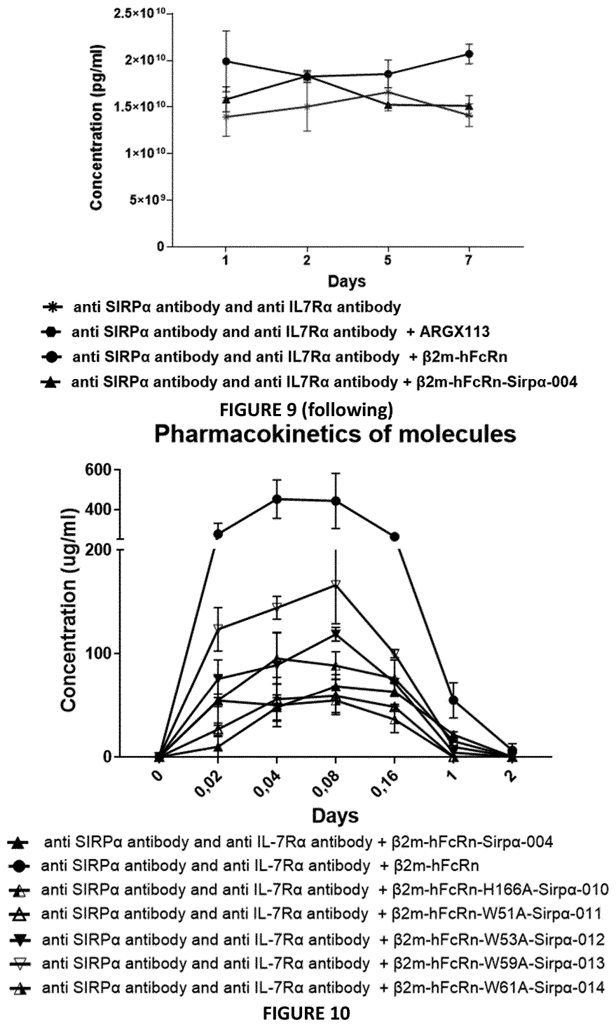

Kinetics study of albumin concentration as shown in FIG. 9 was assessed in presence of FcRn molecules, FcRn mutated molecules and ARGX113.

Immunocompetent 7 weeks old Balb/c mice were intraperitoneally co-injected with one dose of anti SIRPα antibody and anti IL-7Rα antibody at day 0 (25 μg) and one dose of FcRn mutated molecules at day 1 (100 μg or 300 μg). Concentration of albumin in the sera was assessed by ELISA at multiple time points following injection using Mouse Albumin Matched Antibody Pair Kit (ab210890/GR3339694-11/Q6923).

Results: Albumin concentration in presence of FcRn molecules, FcRn mutated molecules and ARGX113 was shown in FIG. 9. No modification of albumin concentration was observed with injection of β2m-hFcRn-SIRPα-004 compared to control group. No modification of albumin was observed with all mutated FcRn molecules: β2m-hFcRn-H166A-Sirpa-010 (100 μg), β2m-hFcRn-W51A-Sirpa-011 (300 μg), β2m-hFcRn-W53A-Sirpa-012 (300 μg), β2m-hFcRn-W59A-Sirpa-013 (300 μg), β2m-hFcRn-W61A-Sirpa-014 (300 μg), β2m-hFcRn-IL7Rα-004 (300 μg) compared to control group and compared to β2m-hFcRn-SIRPα-004. No modification of albumin concentration was observed with injection of ARGX113, β2m-hFcRn and β2m-hFcRn-IL7Rα-004 compared to control group.

### Example 9: Pharmacokinetics of FcRn Mutated Molecules in Mice

Pharmacokinetics of FcRn mutated molecules as shown in FIG. 10 was assessed.

Immunocompetent 7 week old Balb/c mice were intraperitoneally co-injected with one dose of anti SIRPα antibody and anti IL-7Rα antibody at day −1 (25 μg) and one dose of FcRn mutated molecules at day 0 (300 μg). Concentration of FcRn molecules in the sera was assessed by ELISA at multiple time points following injection using anti-@2m (invitrogen #PA5-80367) then serum-containing drugs were added. Detection were performed with biotinylated mouse anti-HIS (MBL #D291-6,006/13052520/OG-HIS) and peroxidase-labeled streptavidin (Jackson immunoresearch; USA; reference 016-030-084) and revealed by conventional methods.

Results: Mutated FcRn molecules concentration was shown in FIG. 10.

At 30 minutes after intraperitoneal injection, β2m-hFcRn-Sirpa-004 was detected in the sera. At 2 hours (0.08 days), the highest concentration was observed. At one day, β2m-hFcRn-Sirpa-004 was not detected in the sera.

The same kinetics was observed with β2m-hFcRn-W51A-Sirpa-011 and β2m-hFcRn-W61A-Sirpa-014 mutated FcRn molecules compared to β2m-hFcRn-Sirpa-004.

A better kinetics was observed with injection of β2m-hFcRn-H166A-Sirpa-010 and β2m-hFcRn-W53A-Sirpa-012 compared to β2m-hFcRn-Sirpa-004. With β2m-hFcRn-H166A-Sirpa-010 and β2m-hFcRn-W53A-Sirpa-012, the concentration was higher at 2 hours than with β2m-hFcRn-Sirpa-004. The same kinetics was observed at one day than β2m-hFcRn-Sirpa-004, namely β2m-hFcRn-H166A-Sirpa-010 and β2m-hFcRn-W53A-Sirpa-012 were not detected.

Even more, a better kinetics was observed with injection of β2m-hFcRn-W59A-Sirpa-013 compared to β2m-hFcRn-Sirpa-004, β2m-hFcRn-H166A-Sirpa-010, β2m-hFcRn-W51A-Sirpa-011, β2m-hFcRn-W53A-Sirpa-012 and β2m-hFcRn-W61A-Sirpa-014. The concentration was higher at 2 hours than all mutated FcRn constructs and β2m-hFcRn-Sirpa-004. The same kinetics at one day was observed compared to β2m-hFcRn-Sirpa-004 and all FcRn mutated constructs.

### Example 10: Pharmacokinetics of Anti-SIRPα Antibody and Anti-IL7Rα Antibody in NHP in Presence of β2m-hFcRn-SIRPα-004

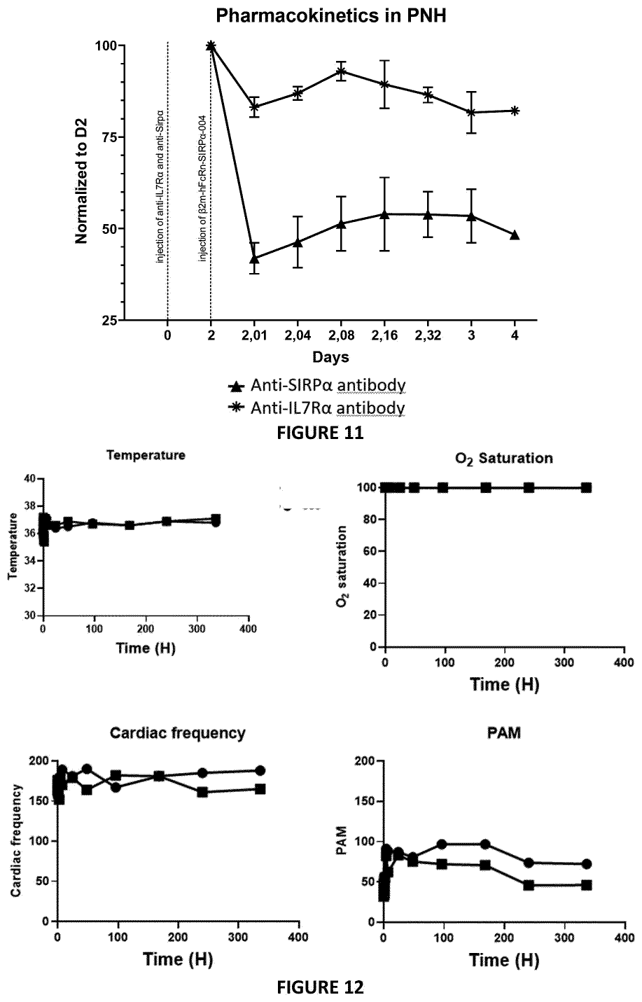

Pharmacokinetics of anti-SIRPα antibody and anti-IL7Rα antibody as shown in FIG. 11 was assessed in presence of β2m-hFcRn-SIRPα-004.

Two non-human primates were intravenously co-injected with one dose of anti-SIRPα antibody and anti-IL7Rα antibody at day 0 at 1 mg/kg and one dose of FcRn molecule: β2m-hFcRn-Sirpa-004 at day 2 at 10 mg/kg. Pharmacokinetics of anti-SIRPα antibody were evaluated by Elisa using anti-idiotype anti SIRPα antibody immobilized then serum-containing drugs and antibodies were added. Detection were performed with mouse anti-human kappa antibody. Donkey anti-mouse PO (JI #715-036-151, lot 104986) was added and revealed by conventional methods.

Pharmacokinetics of anti-IL7Rα antibody were evaluated by Elisa using CD127-Fc (CD127-fc; 306-IR) immobilized then serum-containing drugs and antibodies were added. Detection were performed with mouse anti-human kappa antibody. Donkey anti-mouse PO (JI #715-036-151, lot 104986) was added and revealed by conventional methods.

Results: Pharmacokinetics of anti-IL7Rα antibody and anti-SIRPα antibody in presence of β2m-hFcRn-Sirpa-004 was shown in FIG. 11. Normalized data to D2 was represented.

At day 2, before injection of β2m-hFcRn-SIRPα-004, anti-IL7Rα antibody and anti-SIRPα antibody were detected in both animals. At day 2, 30 minutes after injection of β2m-hFcRn-SIRPα-004, anti-SIRPα antibody kinetics was decreased compared to day 2 before injection. A stabilization at 50% until day 4 was observed.

At day 2, 30 minutes after injection of β2m-hFcRn-SIRPα-004, anti-IL7Rα antibody kinetics was not modified compared to day 2 before injection. A high specificity of β2m-hFcRn-SIRPα-004 action was observed.

### Example 11: Physiological Parameters of NHP after Intravenously Injection of Anti-SIRPα Antibody and Anti-IL7Rα Antibody in Presence of β2m-hFcRn-Sirpa-004

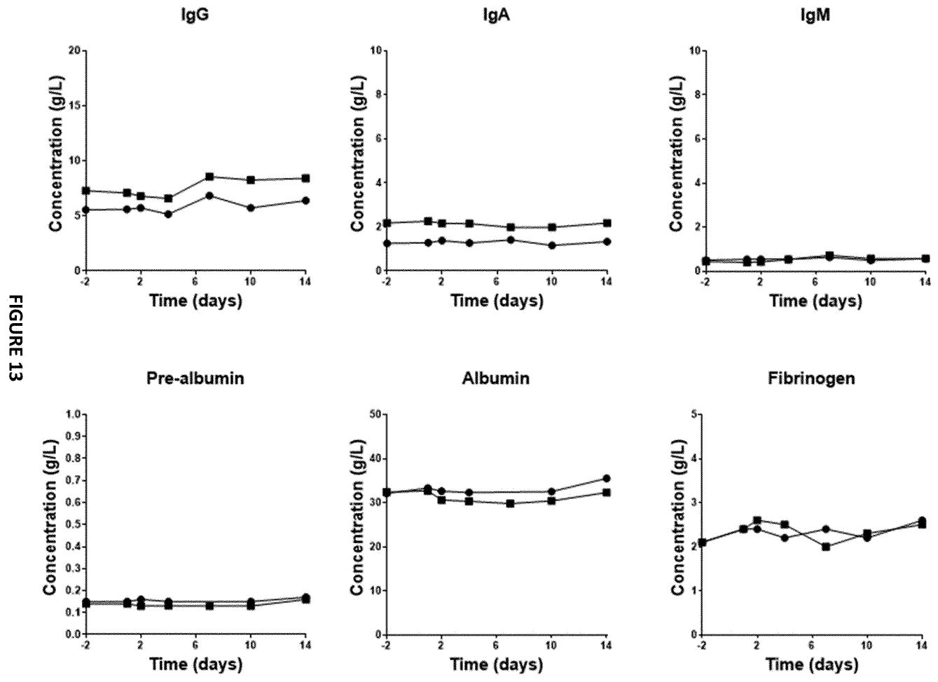

Physiological parameters of NHP as shown in FIG. 12 was assessed in presence of FcRn molecules.

Two non-human primates were intravenously co-injected with one dose of anti-SIRPα antibody and anti-IL7Rα antibody at day 0 at 1 mg/kg and FcRn molecule: β2m-hFcRn-Sirpa-004 at day 2 at 10 mg/kg. Temperature, saturation of O₂, cardiac frequency and PAM (average blood pressure) of NHP were represented in graphs.

Results: The temperature, saturation of O₂, cardiac frequency and PAM (average blood pressure) of NHP in presence of β2m-hFcRn-Sirpa-004 after intravenously injection of anti-SIRPα antibody and anti-IL7Rα antibody were shown in graphs.

Physiological parameters of NHP: temperature, saturation of O₂, cardiac frequency and PAM (average blood pressure) were not modified after intravenously co-injections with one dose of anti-SIRPα antibody and anti-IL7Rα antibody at day 0 at 1 mg/kg and one dose of β2m-hFcRn-Sirpa-004 at day 2 at 10 mg/kg.

### Example 12: Concentration of Proteins in Sera of NHP after Intravenously Injection of Anti-SIRPα Antibody and Anti-IL7Rα Antibody in Presence of β2m-hFcRn-Sirpa-004

Concentration of proteins in sera of NHP as shown in FIG. 13 was assessed in presence of FcRn molecules.

Two non-human primates were intravenously co-injected with anti-SIRPα antibody and anti-IL7Rα antibody at day 0 at 1 mg/kg and one dose of β2m-hFcRn-Sirpa-004 at day 2 at 10 mg/kg. IgG, IgA, IgM, pre-albumin, albumin and fibrinogen concentration (g/L) were measured in NFS vial by conventional method of blood analysis (Analysis laboratory) and values were represented in graphs.

Results: IgG, IgA, IgM, pre-albumin, albumin and fibrinogen concentration (g/L) of NHP in presence of β2m-hFcRn-Sirpa-004 after intravenously injection of anti-SIRPα antibody and anti-IL7Rα antibody were represented in graphs.

IgG, IgA, IgM, pre-albumin, albumin and fibrinogen were not modified after intravenously co-injections with anti-SIRPα antibody and anti-IL7Rα antibody at day 0 at 1 mg/kg and one injection of β2m-hFcRn-Sirpa-004 at day 2 at 10 mg/kg.

### Example 13: Anti-RBD IgG Titers after Immunisation Mice Balb/c Model with Peptide from Viral RBD Protein to Induce Humoral B Cell Response and Treated with β2m-mFcRn-vRBD-004 Molecules

6/7 weeks old Balb/c mice were subcutaneously immunized with emulsion containing two peptides designed to induce humoral B cell response in footpath (from RBD viral protein) and montanide described as enhancer of immune response at day 0 and 7. Each mouse was injected subcutaneously in the left footpad the first week and in the right footpad the second week with 50 ul of the montanide emulsion containing 50 μg of each peptide. Mouse anti-RBD IgG was measured by ELISA using RBD protein (Sinobiological) immobilized then serum of immunized mice were added. Detection were performed with donkey anti-mouse PO (JI #715-036-151, lot 104986) and revealed by conventional methods. Evolution of anti-vRBD IgG titers was represented on graph.

Results: Mouse anti-vRBD antibodies after injection of β2m-mFcRn-vRBD-004 were represented in graphs.

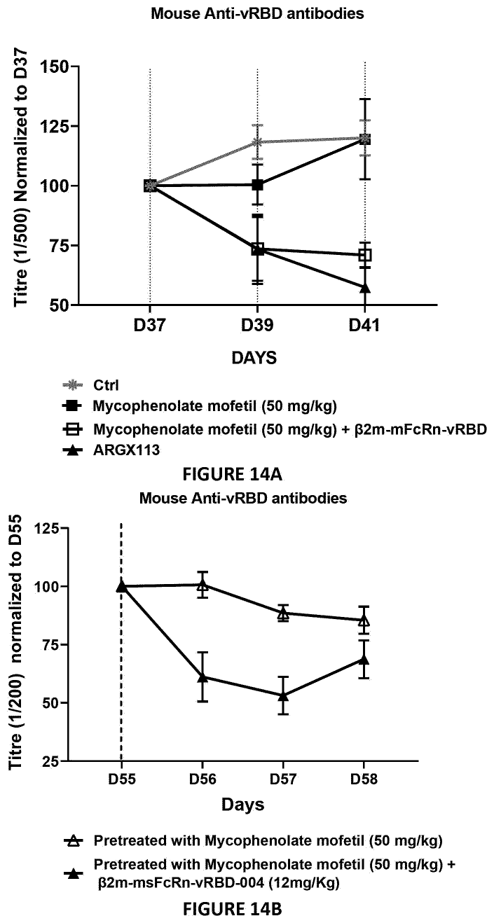

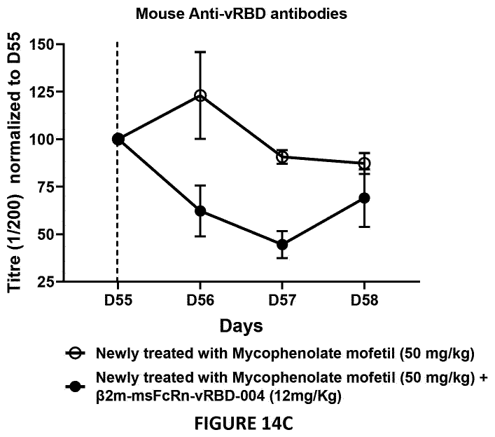

At day 37, after validation of anti-vRBD antibodies production by mice, mice were daily forced-fed with Mycophenolate mofetil at 50 mg/kg and any modification of mouse anti-vRBD antibodies titer was observed compared to control group injected only with emulsion containing montanide and peptides. At day 41, mouse anti-vRBD antibodies titer were decreased in group of mice forced-fed daily with Mycophenolate mofetil at 50 mg/kg and intraperitoneally injected with β2m-mFcRn-vRBD (4 mg/kg) or ARGX113 at day 37, 39 and 41 compared to control group. 50% of decrease was observed compared to control group after two injections of drugs (namely β2m-mFcRn-vRBD or ARGX113) at day 41 (FIG. 14A) (Data were normalized to D37 titer).

Mice were forced-fed daily with Mycophenolate mofetil at 50 mg/kg during several weeks and intraperitoneally injected at D55 with PBS or one dose of β2m-mFcRn-vRBD (12 mg/kg). Mouse anti-vRBD antibodies titer was decreased in group of mice treated intraperitoneally with one dose of β2m-mFcRn-vRBD at 12 mg/kg and forced-fed daily with Mycophenolate mofetil at 50 mg/kg compared to control group. Around 50% of decrease was observed compared to control group after one injection of β2m-mFcRn-vRBD (FIG. 14B).

Mice were newly forced-fed daily with Mycophenolate mofetil at 50 mg/kg or Mycophenolate mofetil at 50 mg/kg and injected intraperitoneally with one dose of β2m-mFcRn-vRBD (12 mg/kg) the same day at D55. Mouse anti-vRBD antibodies titer was decrease in group of mice treated intraperitoneally with one dose of β2m-mFcRn-vRBD at 12 mg/kg and forced-fed with MMF at 50 mg/kg compared to control group. Above 50% of decrease was observed compared to control group after one injection of β2m-mFcRn-vRBD (FIG. 14C).

### Example 14: Pharmacokinetics of Anti-SIRPα Antibody and Anti-IL7Rα Antibody in NHP of in Presence of β2m-hFcRn-Sirpa-004

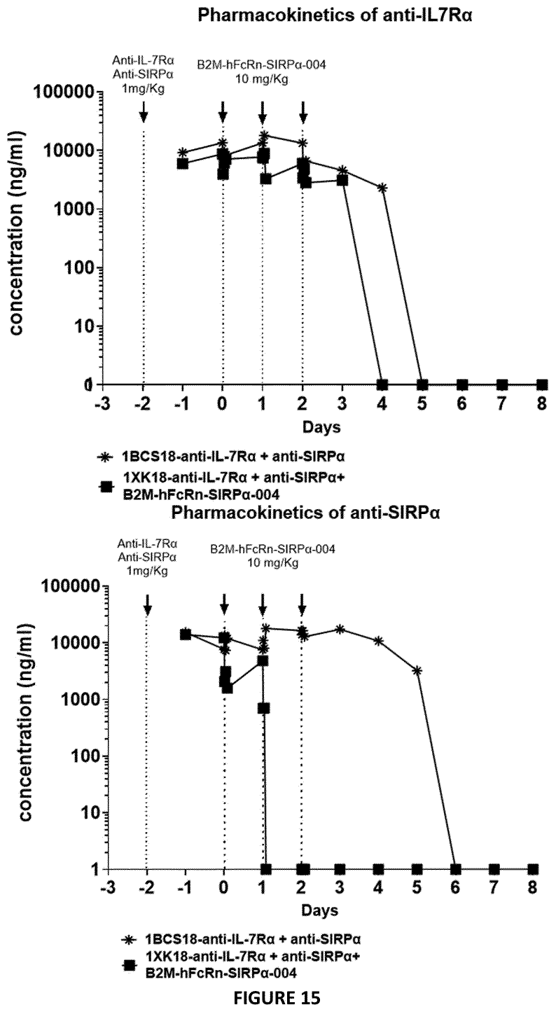

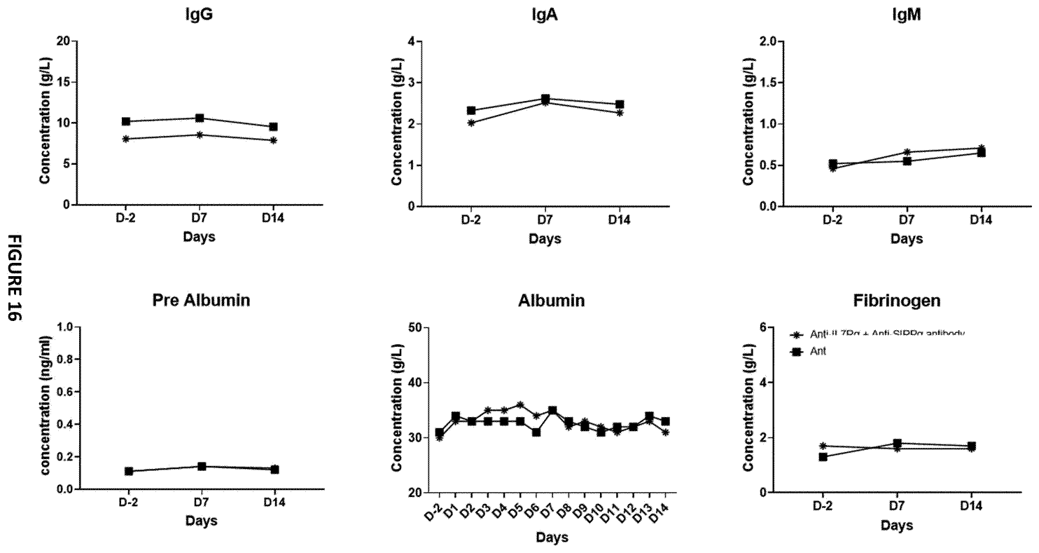

Pharmacokinetics of anti-SIRPα antibody and anti-IL7Rα antibody as shown in FIG. 15 was assessed in presence of FcRn molecules.

Two non-human primates were intravenously co-injected with anti-SIRPα antibody and anti-IL7Rα antibody at day −2 at 1 mg/kg and three intravenously injections of β2m-hFcRn-Sirpa-004 at day 0, 1 and 2 at 10 mg/kg. Pharmacokinetics of anti-SIRPα antibody were evaluated by Elisa using anti-idiotype anti SIRPα antibody immobilized then serum-containing drugs and antibodies were added. Detection were performed with mouse anti-human kappa antibody. Donkey anti-mouse PO (JI #715-036-151, lot 104986) was added and revealed by conventional methods.

Pharmacokinetics of anti-IL7Rα antibody were evaluated by Elisa using CD127-Fc (CD127-fc; 306-IR) immobilized then serum-containing drugs and antibodies were added. Detection were performed with mouse anti-human kappa antibody. Donkey anti-mouse PO (JI #715-036-151, lot 104986) was added and revealed by conventional methods.

Results: Pharmacokinetics of anti-IL7Rα antibody and anti-SIRPα antibody in presence of β2m-hFcRn-Sirpa-004 were shown in FIG. 15.

At day −1, before injection of β2m-hFcRn-SIRPα-004, anti-IL7Rα antibody and anti-SIRPα antibody were detected in both animals.

At day 0, 30 minutes after injection of β2m-hFcRn-SIRPα-004, anti-SIRPα antibody kinetics was decreased compared to control animal without injection of β2m-hFcRn-SIRPα-004. Around 75% of decrease was observed compared to control group 2 hours after the first injections of β2m-hFcRn-SIRPα-004.

At day 1, 30 minutes after second injection of β2m-hFcRn-SIRPα-004, anti-SIRPα antibody kinetics was decreased compared to control animal without injection of β2m-hFcRn-SIRPα-004. Around 100% of decrease was observed compared to control group 1 hours after the second injections of β2m-hFcRn-SIRPα-004.

At day 0, 1, 2, after injection of β2m-hFcRn-SIRPα-004, no modification of the anti-IL7Rα antibody kinetics compared to control animal was observed.

### Example 15: Concentration of Proteins in Sera of NHP Injected Intravenously with One Dose of Anti-SIRPα Antibody and Anti-IL7Rα Antibody and Treated with Three Doses of β2m-hFcRn-Sirpa-004

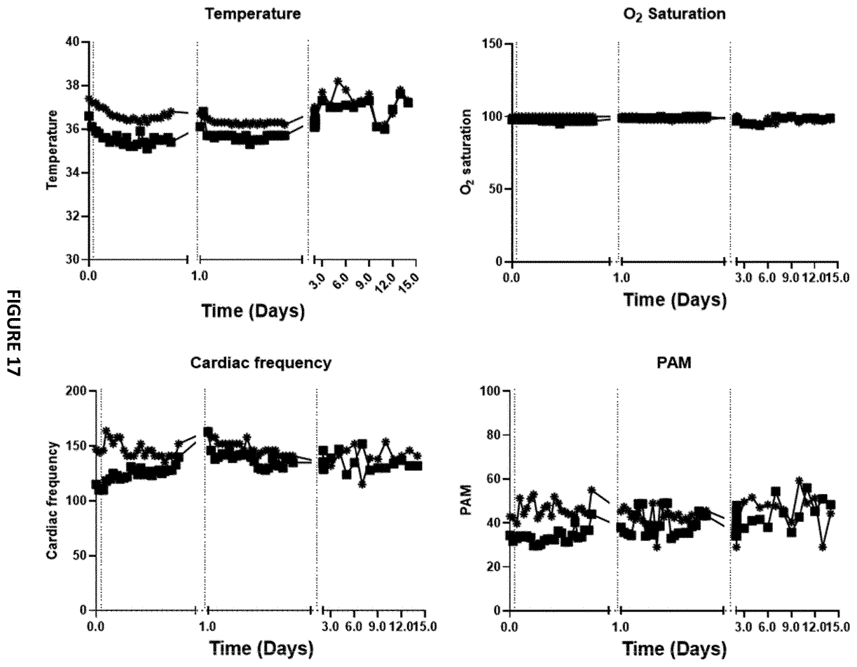

Concentration of proteins in sera of NHP as shown in FIG. 16 was assessed in presence of FcRn molecules.

Two non-human primates were intravenously co-injected with one dose of anti-SIRPα antibody and anti-IL7Rα antibody at day −2 at 1 mg/kg and three doses of β2m-hFcRn-Sirpa-004 at day 0.1 and 2 at 10 mg/kg. IgG, IgA, IgM, pre-albumin, albumin and fibrinogen concentration (g/L) were represented in graphs.

Results: IgG, IgA, IgM, pre-albumin, albumin and fibrinogen concentration (g/L) of NHP in presence of β2m-hFcRn-Sirpa-004 after intravenously injection of anti-SIRPα antibody and anti-IL7Rα antibody were represented in graphs.

IgG, IgA, IgM, pre-albumin, albumin and fibrinogen were not modified after intravenously co-injection with anti-SIRPα antibody and anti-IL7Rα antibody at day 0 at 1 mg/kg and β2m-hFcRn-Sirpa-004 at day 0, 1, 2 at 10 mg/kg.

### Example 16: Temperature, Saturation of O₂, Cardiac Frequency and PAM of NHP Injected Intravenously with One Dose of Anti-SIRPα Antibody and Anti-IL7Rα Antibody and Treated with Three Doses of β2m-hFcRn-Sirpa-004 were Represented

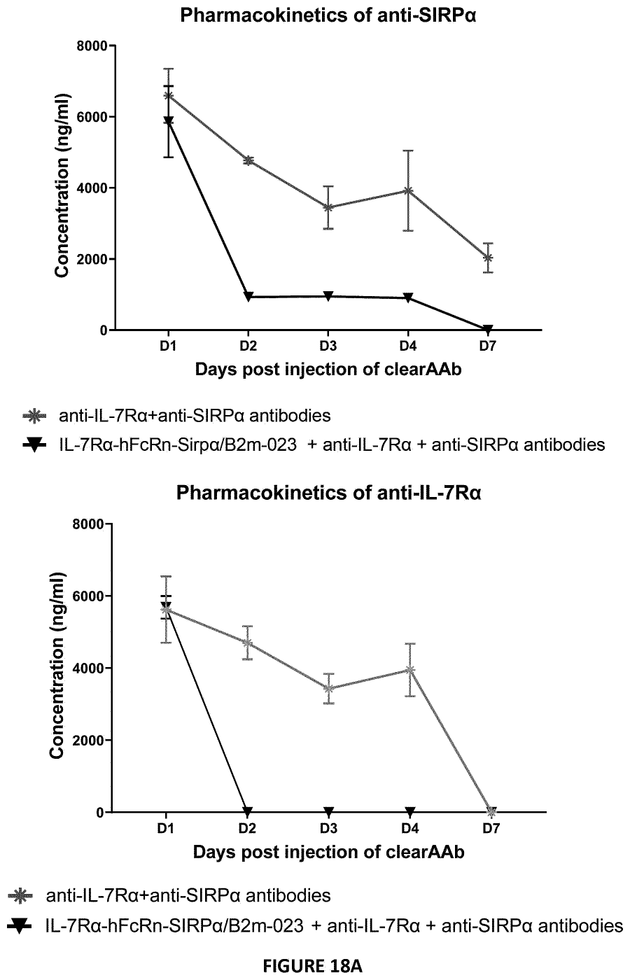

Physiological parameters of NHP as shown in FIG. 17 were assessed in presence of FcRn molecules.

Two non-human primates intravenously co-injected with one dose of anti-SIRPα antibody and anti-IL7Rα antibody at day −2 at 1 mg/kg and three doses of β2m-hFcRn-Sirpa-004 at day 0.1 and 2 at 10 mg/kg. Graph represents temperature, saturation of 02, cardiac frequency and PAM of NHP.

Results: The temperature, saturation of O₂, cardiac frequency and PAM (average blood pressure) of NHP in presence of β2m-hFcRn-Sirpa-004 after intravenously injection of anti-SIRPα antibody and anti-IL7Rα antibody were represented.

Physiological parameters of NHP: temperature, saturation of O₂, cardiac frequency and PAM (average blood pressure) were not modified after an intravenously co-injections with one dose of anti-SIRPα antibody and anti-IL7Rα antibody at day 0 at 1 mg/kg and β2m-hFcRn-Sirpa-004 at day 0.1 and 2 at 10 mg/kg.

### Example 17: Pharmacokinetics of Anti SIRPα Antibody and Anti IL7Rα Antibody in Mice

Pharmacokinetics study of the anti SIRPα antibody and anti-IL7Rα antibody was assessed in presence of bispecific FcRn molecule.

Immunocompetent 7 weeks old Balb/c mice were intraperitoneally co-injected with one dose of anti SIRPα antibody and anti IL7Rα antibody at day 0 (25 ag/injection) and one dose of bispecific FcRn molecule at day 1 (300 ag/injection). Concentration of the anti SIRPα antibody in the sera was assessed by ELISA, at multiple time points following injection, using anti-idiotype-SIRPα antibody immobilized, then serum-containing antibodies and drugs were added. Detection was performed with donkey anti-human HRP (#709-035-149, Jackson Laboratory) and revealed by conventional methods.

Concentration of the anti-IL7Rα antibody in the sera was assessed by ELISA, at multiple time points following injection using CD127-Fc (CD127-fc; 306-IR) immobilized then serum-containing drugs and antibodies were added. Detection was performed with mouse anti-human kappa antibody. Donkey anti-mouse PO (JI #715-036-151, lot 104986) was added and revealed by conventional methods.

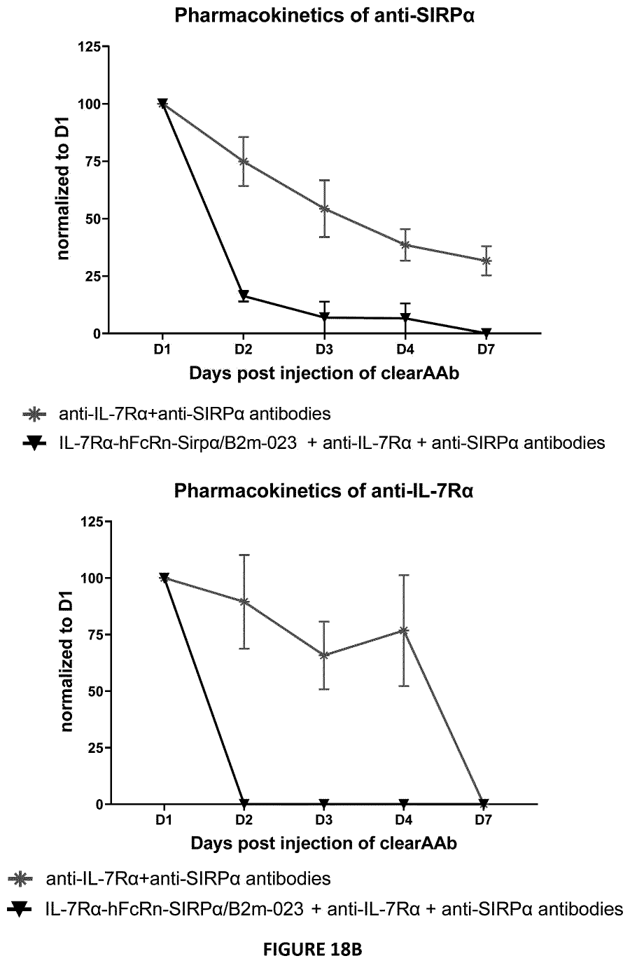

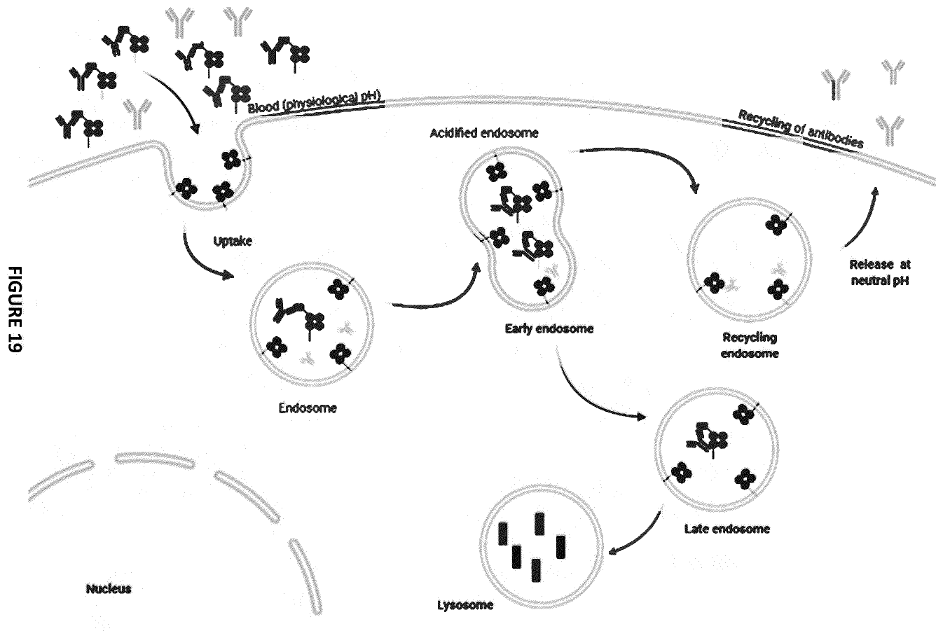

Results: Pharmacokinetics profile of the anti-SIRPα antibody and anti-IL7Rα antibody in presence of bispecific FcRn molecule were shown in FIG. 18.

A decrease of the anti-SIRPα antibody concentration was observed after intraperitoneal injection of IL-7Rα-hFcRn-SIRPα/β2m-023 in comparison to control group injected with anti-SIRPα antibody and anti-IL-7Rα antibody. This decrease kinetics was observed from the first injection and the total elimination kinetics was observed at day 7 compared to anti-SIRPα and anti-IL-7Rα antibodies alone.

A decrease of the anti-IL-7Rα antibody concentration was observed after intraperitoneal injection of IL-7Rα-hFcRn-SIRPα/β2m-023 in comparison to control group injected with anti-SIRPα antibody and anti-IL-7Rα antibody. This decrease kinetics was observed from the first injection and the total elimination kinetics was observed at day 2 compared to anti-SIRPα antibody alone.

Thus, IL-7Rα-hFcRn-SIRPα/β2m-023 bispecific molecule presents a capacity to eliminate both antibodies.

### Example 18: Pharmacokinetics of Humanized Anti-SIRPα Antibody and Humanized Anti-IL-7Rα Antibody in Mice in Presence of Bispecific FcRn Molecules

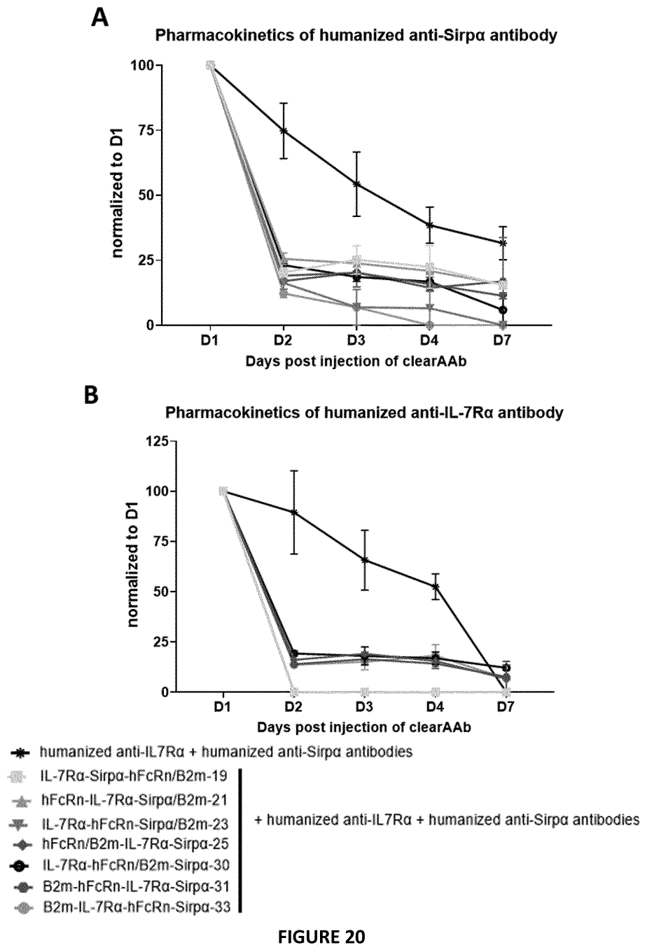

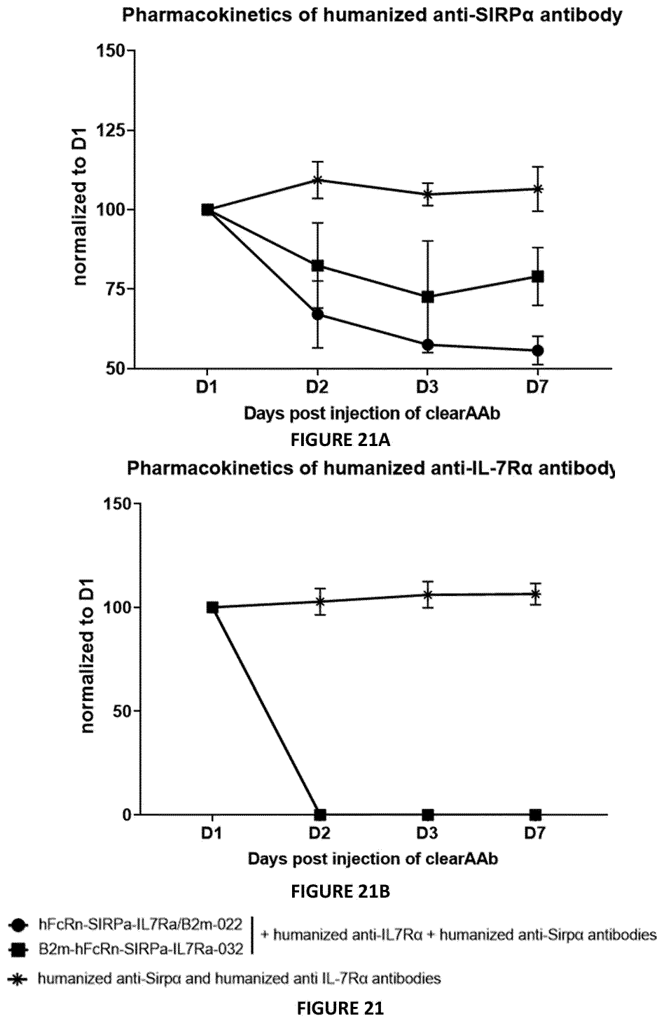

Pharmacokinetics study of the humanized anti-SIRPα antibody or of the humanized anti-IL7-Ra antibody as described in FIGS. 20 and 21 was assessed in presence of FcRn molecules to validate a bispecific model.

Immunocompetent 7 weeks old Balb/c mice were intraperitoneally co-injected with one dose of humanized anti-SIRPα antibody and humanized anti-IL-7Rα antibody at day 0 (25 μg) and one dose of FcRn molecules at day 1 (300 μg). Concentration of the humanized anti-SIRPα antibody in the sera was assessed by ELISA at multiple time points following injection using anti-idiotype SIRPα antibody immobilized, then serum-containing antibodies and drugs were added. Detection was performed with donkey anti-human HRP (#709-035-149, Jackson Laboratory) and revealed by conventional methods.

Concentration of the humanized anti-IL-7Rα antibody in the sera was assessed by ELISA at multiple time points following injection using CD127-Fc (CD127-fc; 306-IR), then serum-containing antibodies and drugs were added. Detection was performed with donkey anti-human HRP (#709-035-149, Jackson Laboratory) and revealed by conventional methods.

Results: Pharmacokinetics profiles of the humanized anti-SIRPα antibody and humanized anti-IL-7Rα antibody in presence of bispecific FcRn molecules are shown in FIGS. 20 and 21.

Humanized anti-IL-7Rα antibody concentration and humanized anti SIRPα antibody concentration were decreased in presence of IL-7Rα-Sirpa-hFcRn/β2m-19, hFcRn-IL-7Rα-Sirpa/β2m-21, IL-7Rα-hFcRn-Sirpa/β2m-23, hFcRn/β2m-IL-7Rα-Sirpa-25, IL-7Rα-hFcRn/β2m-Sirpa-30, β2m-hFcRn-IL-7Rα-Sirpa-31 and β2m-IL-7Rα-hFcRn-Sirpa-33 (FIG. 20) compared to control group.

Humanized anti-IL-7Rα antibody concentration at day 7 was decreased and a partial decrease of anti-SIRPα antibody concentration was shown in presence of hFcRn-SIRPα-IL7Rα/β2m-022 and β2m-hFcRn-SIRPα-IL7Rα-032 (FIG. 21) compared to control group.

A bispecific model was assessed and validated to decrease two specific antibodies instead of one using FcRn bispecific molecules.

### Example 19: Pharmacokinetics of Mouse Anti-vRBD Antibody in Mice in Presence of FcRn Molecules

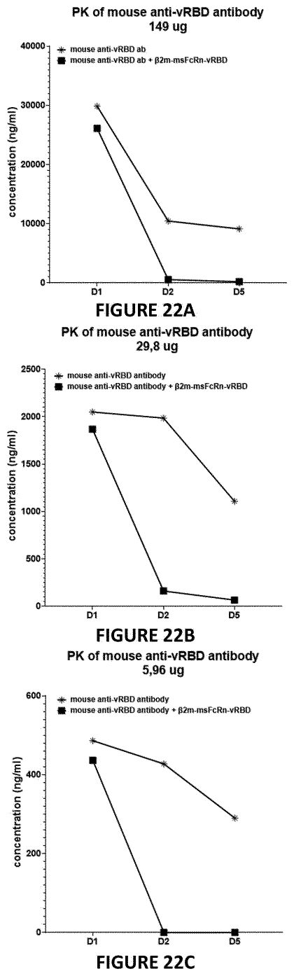

Pharmacokinetics study of the mouse anti-vRBD antibody as described in FIG. 22 was assessed in presence of FcRn molecules.

Immunocompetent 7 weeks old Balb/c mice were intraperitoneally injected with mouse anti-vRBD antibody day 0 (149 μg (A), 29.8 μg (B) and 5.96 μg (C)) and two doses of FcRn molecules at day 1 and 2 (500 μg). Concentration of the mouse anti-vRBD antibody in the sera was assessed by ELISA at multiple time points following injection using vRBD protein immobilized, then serum-containing antibodies and drugs were added. Detection was performed with anti-mouse HRP (#, Jackson Laboratory) and revealed by conventional methods.

Results: Pharmacokinetics profile of the mouse anti-vRBD antibody in presence of FcRn molecules is shown in FIG. 22. Mouse anti-vRBD antibody concentration at day 2 was decreased in presence of β2-msFcRn-vRBD compared to control group.

### Example 20: Pharmacokinetics of Mouse Anti-hDSG3 Antibody in Mice in Presence of FcRn Molecules

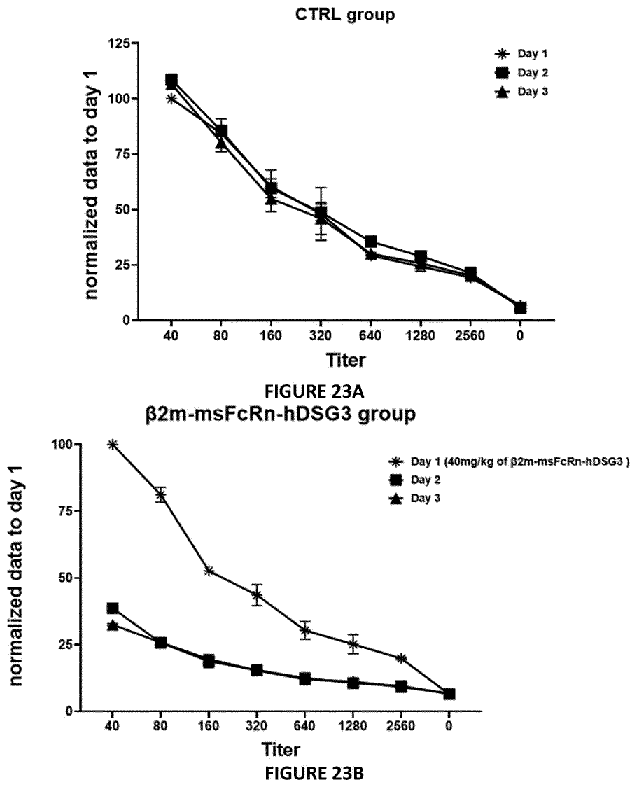

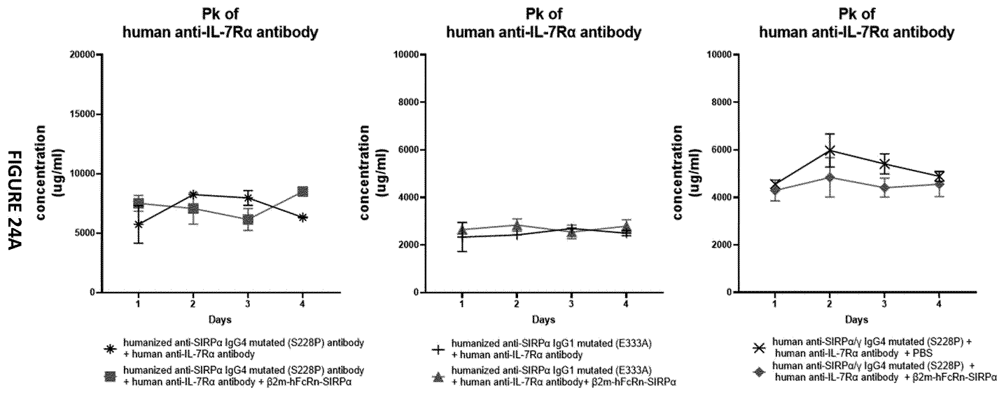

Pharmacokinetics study of the mouse anti hDSG3 antibody as described in FIG. 23 was assessed in presence of FcRn molecules.

Immunocompetent 7 weeks old Balb/c mice were intraperitoneally injected with 200 μl of sera containing mouse anti-hDSG3 antibody at day 0 and one dose of FcRn molecules at day 1 (1000 μg). Concentration of the mouse anti-hDSG3 antibody in the sera was assessed by ELISA at multiple time points following injection using hDSG3 protein immobilized, then serum-containing antibodies and drugs were added. Detection was performed with anti-mouse HRP (#, Jackson Laboratory) and revealed by conventional methods.

Results: Pharmacokinetics profile of the mouse anti hDSG3 antibody in presence of FcRn molecules is shown in FIG. 23. Mouse anti-hDSG3 antibody concentration at day 2 was decreased with injection of β2-msFcRn-hDSG3 compared to control group.

### Example 21: Pharmacokinetics of Anti-SIRPα Antibody and Anti-IL-7Rα Antibody in Mice in Presence of FcRn Molecules

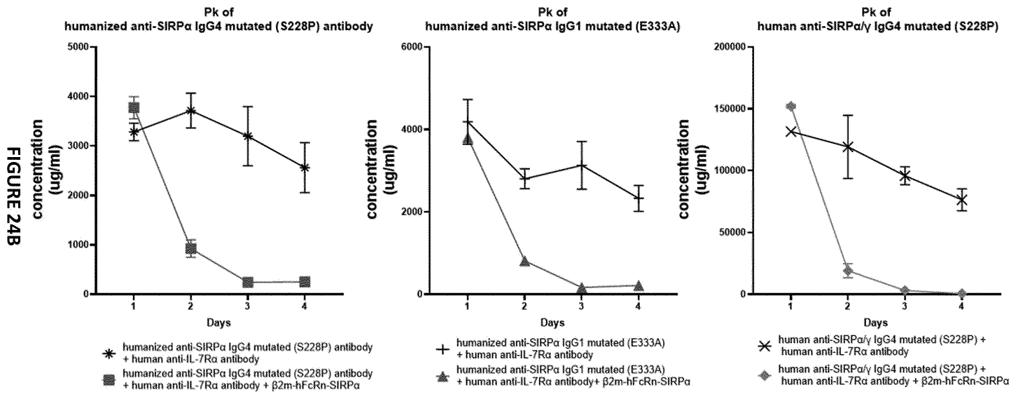

Pharmacokinetics study of the anti-SIRPα antibody as described in FIG. 24 was assessed in presence of FcRn molecules to validate an elimination of different IgG isotypes.

Immunocompetent 7 weeks old Balb/c mice were intraperitoneally co-injected with one dose of anti-SIRPα antibody and anti-IL-7Rα antibody at day 0 (25 μg) and two doses of FcRn molecules at day 1 and 2 (500 μg).

Concentration of the anti-SIRPα antibody in the sera was assessed by ELISA at multiple time points following injection using anti-idiotype SIRPα antibody immobilized or SIRPγ, then serum-containing antibodies and drugs were added. Detection was performed with donkey anti-human HRP (#709-035-149, Jackson Laboratory) and revealed by conventional methods.

Concentration of the anti-IL-7Rα antibody in the sera was assessed by ELISA at multiple time points following injection using CD127-Fc (CD127-fc; 306-IR), then serum-containing antibodies and drugs were added. Detection was performed with donkey anti-human HRP (#709-035-149, Jackson Laboratory) and revealed by conventional methods.

Results: Pharmacokinetics profiles of the anti-IL-7Rα antibody (A) and anti-SIRPα antibody (B) in presence of FcRn molecules are shown in FIG. 24.

Concentration of human anti-IL-7Rα antibody in presence of β2m-hFcRn-SIRPα was not modified in each group compared to control group (A). A total decrease of humanized anti-SIRPα IgG4 mutated (S228P) antibody in presence of β2m-hFcRn-SIRPα was observed compared to control group at day 3 (B). A total decrease of humanized anti-SIRPα IgG1 mutated (E333A) antibody in presence of β2m-hFcRn-SIRPα was also observed compared to control group at day 3 (B). In addition, a total decrease of human anti-SIRPα/γ IgG4mutated (S228P) antibody in presence of β2m-hFcRn-SIRPα was observed compared to control group at day 3 (B).

Accordingly, IgG1 or IgG4 antibody can be eliminated by hFcRn molecule, and a dual antibody SIRPα/γ can be also eliminated by β2m-hFcRn-SIRPα molecule.

### Materials and Methods

### Expression and Purification of Molecules

The FcRn molecules were built by fusing the human FcRn heavy chain (hFcRnH) either N or C-terminally to an antigen (either hSIRPα, hCD127 or a peptide) via a (G4S)₃ flexible linker. In the case of single-chain constructs, hβ2M was in addition fused N-terminally to hFcRnH via a flexible (G4S)₃ linker. A C-terminal His-tag was added C-terminally to the different constructs in order to easily purify the molecules by IMAC.

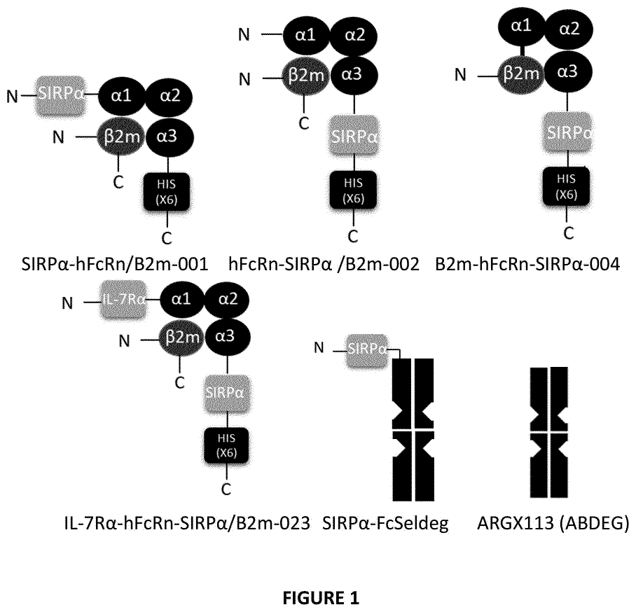

The FcRn molecules are schematically described in FIG. 1. The sequences of the molecules are disclosed in the sequence listing.

| SEQ ID | | |
|---|---|---|
| NO | Name | Sequence |
| 2 | hFcRn | AESHLSLLYHLTAVSSPAPGTPAFWVSGWLGPQQYLSYNSLRGEAEPCGAWVWEN |
| | without | QVSWYWEKETTDLRIKEKLFLEAFKALGGKGPYTLQGLLGCELGPDNTSVPTAKFALN |
| | signal | GEEFMNFDLKQGTWGGDWPEALAISQRWQQQDKAANKELTFLLFSCPHRLREHLE |
| | peptide and | RGRGNLEWKEPPSMRLKARPSSPGFSVLTCSAFSFYPPELQLRFLRNGLAAGTGQGD |
| | including | FGPNSDGSFHASSSLTVKSGDEHHYCCIVQHAGLAQPLRVELESPAKSS |
| | regions | |
| | alpha1, | |
| | alpha2 and | |
| | alpha3 | |
| 4 | B2m | IQRTPKIQVYSRHPAENGKSNFLNCYVSGFHPSDIEVDLLKNGERIEKVEHSDLSFSKD |
| | without | WSFYLLYYTEFTPTEKDEYACRVNHVTLSQPKIVKWDRDM |
| | signal | |
| | peptide | |
| 5 | SIRPα- | EEELQVIQPDKSVLVAAGETATLRCTATSLIPVGPIQWFRGAGPGRELIYNQKEGHFP |
| | hFcRn | RVTTVSDLTKRNNMDFSIRIGNITPADAGTYYCVKFRKGSPDDVEFKSGAGTELSVRA |
| | | KPSAPVVSGPAARATPQHTVSFTCESHGFSPRDITLKWFKNGNELSDFQTNVDPVGE |
| | | SVSYSIHSTAKVVLTREDVHSQVICEVAHVTLQGDPLRGTANLSETIRVPPTLEVTQQP |
| | | VRAENQVNVTCQVRKFYPQRLQLTWLENGNVSRTETASTVTENKDGTYNWMSWL |
| | | LVNVSAHRDDVKLTCQVEHDGQPAVSKSHDLKVSAHPKEQGSNTAAENTGSNERNI |
| | | YGGGGSGGGGSGGGGSAESHLSLLYHLTAVSSPAPGTPAFWVSGWLGPQQYLSYNS |
| | | LRGEAEPCGAWVWENQVSWYWEKETTDLRIKEKLFLEAFKALGGKGPYTLQGLLGC |
| | | ELGPDNTSVPTAKFALNGEEFMNFDLKQGTWGGDWPEALAISQRWQQQDKAANK |
| | | ELTFLLFSCPHRLREHLERGRGNLEWKEPPSMRLKARPSSPGFSVLTCSAFSFYPPELQL |
| | | RFLRNGLAAGTGQGDFGPNSDGSFHASSSLTVKSGDEHHYCCIVQHAGLAQPLRVEL |
| | | ESPAKSSAAAHHHHHH |
| 6 | hFcRn- | AESHLSLLYHLTAVSSPAPGTPAFWVSGWLGPQQYLSYNSLRGEAEPCGAWVWEN |
| | SIRPα | QVSWYWEKETTDLRIKEKLFLEAFKALGGKGPYTLQGLLGCELGPDNTSVPTAKFALN |
| | | GEEFMNFDLKQGTWGGDWPEALAISQRWQQQDKAANKELTFLLFSCPHRLREHLE |
| | | RGRGNLEWKEPPSMRLKARPSSPGFSVLTCSAFSFYPPELQLRFLRNGLAAGTGQGD |
| | | FGPNSDGSFHASSSLTVKSGDEHHYCCIVQHAGLAQPLRVELESPAKSSGGGGSGGG |
| | | GSGGGGSEEELQVIQPDKSVLVAAGETATLRCTATSLIPVGPIQWFRGAGPGRELIYN |
| | | QKEGHFPRVTTVSDLTKRNNMDFSIRIGNITPADAGTYYCVKFRKGSPDDVEFKSGA |
| | | GTELSVRAKPSAPVVSGPAARATPQHTVSFTCESHGFSPRDITLKWFKNGNELSDFQT |
| | | NVDPVGESVSYSIHSTAKVVLTREDVHSQVICEVAHVTLQGDPLRGTANLSETIRVPPT |
| | | LEVTQQPVRAENQVNVTCQVRKFYPQRLQLTWLENGNVSRTETASTVTENKDGTYN |
| | | WMSWLLVNVSAHRDDVKLTCQVEHDGQPAVSKSHDLKVSAHPKEQGSNTAAENT |
| | | GSNERNIYAAAHHHHHH |
| 7 | β2m-hFcRn- | IQRTPKIQVYSRHPAENGKSNFLNCYVSGFHPSDIEVDLLKNGERIEKVEHSDLSFSKD |
| | Sirpα-004 | WSFYLLYYTEFTPTEKDEYACRVNHVTLSQPKIVKWDRDMGGGGSGGGGSGGGGS |
| | | AESHLSLLYHLTAVSSPAPGTPAFWVSGWLGPQQYLSYNSLRGEAEPCGAWVWEN |
| | | QVSWYWEKETTDLRIKEKLFLEAFKALGGKGPYTLQGLLGCELGPDNTSVPTAKFALN |
| | | GEEFMNFDLKQGTWGGDWPEALAISQRWQQQDKAANKELTFLLFSCPHRLREHLE |
| | | RGRGNLEWKEPPSMRLKARPSSPGFSVLTCSAFSFYPPELQLRFLRNGLAAGTGQGD |
| | | FGPNSDGSFHASSSLTVKSGDEHHYCCIVQHAGLAQPLRVELESPAKSSGGGGSGGG |
| | | GSGGGGSEEELQVIQPDKSVLVAAGETATLRCTATSLIPVGPIQWFRGAGPGRELIYN |
| | | QKEGHFPRVTTVSDLTKRNNMDFSIRIGNITPADAGTYYCVKFRKGSPDDVEFKSGA |
| | | GTELSVRAKPSAPVVSGPAARATPQHTVSFTCESHGFSPRDITLKWFKNGNELSDFQT |
| | | NVDPVGESVSYSIHSTAKVVLTREDVHSQVICEVAHVTLQGDPLRGTANLSETIRVPPT |
| | | LEVTQQPVRAENQVNVTCQVRKFYPQRLQLTWLENGNVSRTETASTVTENKDGTYN |
| | | WMSWLLVNVSAHRDDVKLTCQVEHDGQPAVSKSHDLKVSAHPKEQGSNTAAENT |
| | | GSNERNIYAAAHHHHHH |
| 8 | SIRPα- | EEELQVIQPDKSVLVAAGETATLRCTATSLIPVGPIQWFRGAGPGRELIYNQKEGHFP |
| | FcSeldeg | RVTTVSDLTKRNNMDFSIRIGNITPADAGTYYCVKFRKGSPDDVEFKSGAGTELSVRA |
| | Chain 1 | KPSAPVVSGPAARATPQHTVSFTCESHGFSPRDITLKWFKNGNELSDFQTNVDPVGE |
| | | SVSYSIHSTAKVVLTREDVHSQVICEVAHVTLQGDPLRGTANLSETIRVPPTLEVTQQP |
| | | VRAENQVNVTCQVRKFYPQRLQLTWLENGNVSRTETASTVTENKDGTYNWMSWL |
| | | LVNVSAHRDDVKLTCQVEHDGQPAVSKSHDLKVSAHPKEQGSNTAAENTGSNERNI |
| | | YIEGRMDPKSSDKTHTCPPCPAPELLRGPSVFLFPPKPKDTLYITREPEVTCVVVDVSH |
| | | EDPEVKFNWYVDGVEVHNAKTKPREEQYNSTYRVVSVLTVLHQDWLNGKEYKCKVS |
| | | NKARPAPIEKTISKAKGQPREPQVTTLPPSRDELTKNQVSLTCLVKGFYPSDIAVEWES |
| | | NGQPENNYKTFPPVLDSDGSFFLYSKLTVDKSRWQQGNVFSCSVMHEALKFHYTQK |
| | | SLSLSPGK |
| 9 | Fc Seldeg | VEPKSSDKTHTCPPCPAPELLRGPSVFLFPPKPKDTLYITREPEVTCVVVDVSHEDPEVK |
| | Chain 2 | FNWYVDGVEVHNAKTKPREEQYNSTYRVVSVLTVLHQDWLNGKEYKCKVSNKARP |
| | | APIEKTISKAKGQPREPQVYTLPPSRDELTKNQVHLTCLVKGFYPSDIAVEWESNGQP |
| | | ENNYKTTPPVLDSDGSFALYSKLTVDKSRWQQGNVFSCSVMHEALKFHYTQKSLSLS |
| | | PGK |
| 10 | ARGX113 | DKTHTCPPCPAPELLGGPSVFLFPPKPKDTLYITREPEVTCVVVDVSHEDPEVKFNWY |
| | | VDGVEVHNAKTKPREEQYNSTYRVVSVLTVLHQDWLNGKEYKCKVSNKALPAPIEKT |
| | | ISKAKGQPREPQVYTLPPSRDELTKNQVSLTCLVKGFYPSDIAVEWESNGQPENNYKT |
| | | TPPVLDSDGSFFLYSKLTVDKSRWQQGNVFSCSVMHEALKFHYTQKSLSLSPGK |
| 11 | β2m-hFcRn- | IQRTPKIQVYSRHPAENGKSNFLNCYVSGFHPSDIEVDLLKNGERIEKVEHSDLSFSKD |
| | ILRα-004 | WSFYLLYYTEFTPTEKDEYACRVNHVTLSQPKIVKWDRDMGGGGSGGGGSGGGGS |
| | | AESHLSLLYHLTAVSSPAPGTPAFWVSGWLGPQQYLSYNSLRGEAEPCGAWVWEN |
| | | QVSWYWEKETTDLRIKEKLFLEAFKALGGKGPYTLQGLLGCELGPDNTSVPTAKFALN |
| | | GEEFMNFDLKQGTWGGDWPEALAISQRWQQQDKAANKELTFLLFSCPHRLREHLE |
| | | RGRGNLEWKEPPSMRLKARPSSPGFSVLTCSAFSFYPPELQLRFLRNGLAAGTGQGD |
| | | FGPNSDGSFHASSSLTVKSGDEHHYCCIVQHAGLAQPLRVELESPAKSSGGGGSGGG |
| | | GSGGGGSESGYAQNGDLEDAELDDYSFSCYSQLEVNGSQHSLTCAFEDPDVNITNLE |
| | | FEICGALVEVKCLNFRKLQEIYFIETKKFLLIGKSNICVKVGEKSLTCKKIDLTTIVKPEAPF |
| | | DLSVVYREGANDFVVTFNTSHLQKKYVKVLMHDVAYRQEKDENKWTHVNLSSTKLT |
| | | LLQRKLQPAAMYEIKVRSIPDHYFKGFWSEWSPSYYFRTPEINNSSGEMDAAAHHHH |
| | | HH |
| 12 | β2m-hFcRn- | IQRTPKIQVYSRHPAENGKSNFLNCYVSGFHPSDIEVDLLKNGERIEKVEHSDLSFSKD |
| | Sirpα- | WSFYLLYYTEFTPTEKDEYACRVNHVTLSQPKIVKWDRDMGGGGSGGGGSGGGGS |
| | H116A-010 | AESHLSLLYHLTAVSSPAPGTPAFWVSGWLGPQQYLSYNSLRGEAEPCGAWVWEN |
| | | QVSWYWEKETTDLRIKEKLFLEAFKALGGKGPYTLQGLLGCELGPDNTSVPTAKFALN |
| | | GEEFMNFDLKQGTWGGDWPEALAISQRWQQQDKAANKELTFLLFSCPHRLREALE |
| | | RGRGNLEWKEPPSMRLKARPSSPGFSVLTCSAFSFYPPELQLRFLRNGLAAGTGQGD |
| | | FGPNSDGSFHASSSLTVKSGDEHHYCCIVQHAGLAQPLRVELESPAKSSGGGGSGGG |
| | | GSGGGGSEEELQVIQPDKSVLVAAGETATLRCTATSLIPVGPIQWFRGAGPGRELIYN |
| | | QKEGHFPRVTTVSDLTKRNNMDFSIRIGNITPADAGTYYCVKFRKGSPDDVEFKSGA |
| | | GTELSVRAKPSAPVVSGPAARATPQHTVSFTCESHGFSPRDITLKWFKNGNELSDFQT |
| | | NVDPVGESVSYSIHSTAKVVLTREDVHSQVICEVAHVTLQGDPLRGTANLSETIRVPPT |
| | | LEVTQQPVRAENQVNVTCQVRKFYPQRLQLTWLENGNVSRTETASTVTENKDGTYN |
| | | WMSWLLVNVSAHRDDVKLTCQVEHDGQPAVSKSHDLKVSAHPKEQGSNTAAENT |
| | | GSNERNIYAAAHHHHHH |
| 13 | β2m-hFcRn- | IQRTPKIQVYSRHPAENGKSNFLNCYVSGFHPSDIEVDLLKNGERIEKVEHSDLSFSKD |
| | Sirpα- | WSFYLLYYTEFTPTEKDEYACRVNHVTLSQPKIVKWDRDMGGGGSGGGGSGGGGS |
| | W51A-011 | AESHLSLLYHLTAVSSPAPGTPAFWVSGWLGPQQYLSYNSLRGEAEPCGAAVWENQ |
| | | VSWYWEKETTDLRIKEKLFLEAFKALGGKGPYTLQGLLGCELGPDNTSVPTAKFALNG |
| | | EEFMNFDLKQGTWGGDWPEALAISQRWQQQDKAANKELTFLLFSCPHRLREHLER |
| | | GRGNLEWKEPPSMRLKARPSSPGFSVLTCSAFSFYPPELQLRFLRNGLAAGTGQGDF |
| | | GPNSDGSFHASSSLTVKSGDEHHYCCIVQHAGLAQPLRVELESPAKSSGGGGSGGGG |
| | | SGGGGSEEELQVIQPDKSVLVAAGETATLRCTATSLIPVGPIQWFRGAGPGRELIYNQ |
| | | KEGHFPRVTTVSDLTKRNNMDFSIRIGNITPADAGTYYCVKFRKGSPDDVEFKSGAGT |
| | | ELSVRAKPSAPVVSGPAARATPQHTVSFTCESHGFSPRDITLKWFKNGNELSDFQTN |
| | | VDPVGESVSYSIHSTAKVVLTREDVHSQVICEVAHVTLQGDPLRGTANLSETIRVPPTL |
| | | EVTQQPVRAENQVNVTCQVRKFYPQRLQLTWLENGNVSRTETASTVTENKDGTYN |
| | | WMSWLLVNVSAHRDDVKLTCQVEHDGQPAVSKSHDLKVSAHPKEQGSNTAAENT |
| | | GSNERNIYAAAHHHHHH |
| 14 | β2m-hFcRn- | IQRTPKIQVYSRHPAENGKSNFLNCYVSGFHPSDIEVDLLKNGERIEKVEHSDLSFSKD |
| | Sirpα- | WSFYLLYYTEFTPTEKDEYACRVNHVTLSQPKIVKWDRDMGGGGSGGGGSGGGGS |
| | W53A-012 | AESHLSLLYHLTAVSSPAPGTPAFWVSGWLGPQQYLSYNSLRGEAEPCGAWVAENQ |
| | | VSWYWEKETTDLRIKEKLFLEAFKALGGKGPYTLQGLLGCELGPDNTSVPTAKFALNG |
| | | EEFMNFDLKQGTWGGDWPEALAISQRWQQQDKAANKELTFLLFSCPHRLREHLER |
| | | GRGNLEWKEPPSMRLKARPSSPGFSVLTCSAFSFYPPELQLRFLRNGLAAGTGQGDF |
| | | GPNSDGSFHASSSLTVKSGDEHHYCCIVQHAGLAQPLRVELESPAKSSGGGGSGGGG |
| | | SGGGGSEEELQVIQPDKSVLVAAGETATLRCTATSLIPVGPIQWFRGAGPGRELIYNQ |
| | | KEGHFPRVTTVSDLTKRNNMDFSIRIGNITPADAGTYYCVKFRKGSPDDVEFKSGAGT |
| | | ELSVRAKPSAPVVSGPAARATPQHTVSFTCESHGFSPRDITLKWFKNGNELSDFQTN |
| | | VDPVGESVSYSIHSTAKVVLTREDVHSQVICEVAHVTLQGDPLRGTANLSETIRVPPTL |
| | | EVTQQPVRAENQVNVTCQVRKFYPQRLQLTWLENGNVSRTETASTVTENKDGTYN |
| | | WMSWLLVNVSAHRDDVKLTCQVEHDGQPAVSKSHDLKVSAHPKEQGSNTAAENT |
| | | GSNERNIYAAAHHHHHH |
| 15 | β2m-hFcRn- | IQRTPKIQVYSRHPAENGKSNFLNCYVSGFHPSDIEVDLLKNGERIEKVEHSDLSFSKD |
| | Sirpα- | WSFYLLYYTEFTPTEKDEYACRVNHVTLSQPKIVKWDRDMGGGGSGGGGSGGGGS |
| | W59A-013 | AESHLSLLYHLTAVSSPAPGTPAFWVSGWLGPQQYLSYNSLRGEAEPCGAWVWEN |
| | | QVSAYWEKETTDLRIKEKLFLEAFKALGGKGPYTLQGLLGCELGPDNTSVPTAKFALN |
| | | GEEFMNFDLKQGTWGGDWPEALAISQRWQQQDKAANKELTFLLFSCPHRLREHLE |
| | | RGRGNLEWKEPPSMRLKARPSSPGFSVLTCSAFSFYPPELQLRFLRNGLAAGTGQGD |
| | | FGPNSDGSFHASSSLTVKSGDEHHYCCIVQHAGLAQPLRVELESPAKSSGGGGSGGG |
| | | GSGGGGSEEELQVIQPDKSVLVAAGETATLRCTATSLIPVGPIQWFRGAGPGRELIYN |
| | | QKEGHFPRVTTVSDLTKRNNMDFSIRIGNITPADAGTYYCVKFRKGSPDDVEFKSGA |
| | | GTELSVRAKPSAPVVSGPAARATPQHTVSFTCESHGFSPRDITLKWFKNGNELSDFQT |
| | | NVDPVGESVSYSIHSTAKVVLTREDVHSQVICEVAHVTLQGDPLRGTANLSETIRVPPT |
| | | LEVTQQPVRAENQVNVTCQVRKFYPQRLQLTWLENGNVSRTETASTVTENKDGTYN |
| | | WMSWLLVNVSAHRDDVKLTCQVEHDGQPAVSKSHDLKVSAHPKEQGSNTAAENT |
| | | GSNERNIYAAAHHHHHH |
| 16 | β2m-hFcRn- | IQRTPKIQVYSRHPAENGKSNFLNCYVSGFHPSDIEVDLLKNGERIEKVEHSDLSFSKD |
| | Sirpα- | WSFYLLYYTEFTPTEKDEYACRVNHVTLSQPKIVKWDRDMGGGGSGGGGSGGGGS |
| | W61A-014 | AESHLSLLYHLTAVSSPAPGTPAFWVSGWLGPQQYLSYNSLRGEAEPCGAWVWEN |
| | | QVSWYAEKETTDLRIKEKLFLEAFKALGGKGPYTLQGLLGCELGPDNTSVPTAKFALN |
| | | GEEFMNFDLKQGTWGGDWPEALAISQRWQQQDKAANKELTFLLFSCPHRLREHLE |
| | | RGRGNLEWKEPPSMRLKARPSSPGFSVLTCSAFSFYPPELQLRFLRNGLAAGTGQGD |
| | | FGPNSDGSFHASSSLTVKSGDEHHYCCIVQHAGLAQPLRVELESPAKSSGGGGSGGG |
| | | GSGGGGSEEELQVIQPDKSVLVAAGETATLRCTATSLIPVGPIQWFRGAGPGRELIYN |
| | | QKEGHFPRVTTVSDLTKRNNMDFSIRIGNITPADAGTYYCVKFRKGSPDDVEFKSGA |
| | | GTELSVRAKPSAPVVSGPAARATPQHTVSFTCESHGFSPRDITLKWFKNGNELSDFQT |
| | | NVDPVGESVSYSIHSTAKVVLTREDVHSQVICEVAHVTLQGDPLRGTANLSETIRVPPT |
| | | LEVTQQPVRAENQVNVTCQVRKFYPQRLQLTWLENGNVSRTETASTVTENKDGTYN |
| | | WMSWLLVNVSAHRDDVKLTCQVEHDGQPAVSKSHDLKVSAHPKEQGSNTAAENT |
| | | GSNERNIYAAAHHHHHH |
| 17 | B2m- | IQKTPQIQVYSRHPPENGKPNILNCYVTQFHPPHIEIQMLKNGKKIPKVEMSDMSFSK |
| | mFcRn- | DWSFYILAHTEFTPTETDTYACRVKHASMAEPKTVYWDRDMGGGGGGGGSGGG |
| | vRBD | GSSETRPPLMYHLTAVSNPSTGLPSFWATGWLGPQQYLTYNSLRQEADPCGAWM |
| | | WENQVSWYWEKETTDLKSKEQLFLEALKTLEKILNGTYTLQGLLGCELASDNSSVPTA |
| | | VFALNGEEFMKFNPRIGNWTGEWPETEIVANLWMKQPDAARKESEFLLNSCPERLL |
| | | GHLERGRRNLEWKEPPSMRLKARPGNSGSSVLTCAAFSFYPPELKFRFLRNGLASGSG |
| | | NCSTGPNGDGSFHAWSLLEVKRGDEHHYQCQVEHEGLAQPLTVDLDSSARSSGGG |
| | | GSGGGGSGGGGSGSRVQPTESIVRFPNITNLCPFGEVFNATRFASVYAWNRKRISNC |
| | | VADYSVLYNSASFSTFKCYGVSPTKLNDLCFTNVYADSFVIRGDEVRQIAPGQTGKIAD |
| | | YNYKLPDDFTGCVIAWNSNNLDSKVGGNYNYLYRLFRKSNLKPFERDISTEIYQAGST |
| | | PCNGVEGFNCYFPLQSYGFQPTNGVGYQPYRVVVLSFELLHAPATVCGPKKSTNLVK |
| | | NKCVNFAAAHHHHHH |
| 18 | IL-7Rα- | ESGYAQNGDLEDAELDDYSFSCYSQLEVNGSQHSLTCAFEDPDVNITNLEFEICGALV |
| | hFcRn- | EVKCLNFRKLQEIYFIETKKFLLIGKSNICVKVGEKSLTCKKIDLTTIVKPEAPFDLSVVYRE |
| | SIRPα | GANDFVVTFNTSHLQKKYVKVLMHDVAYRQEKDENKWTHVNLSSTKLTLLQRKLQP |
| | | AAMYEIKVRSIPDHYFKGFWSEWSPSYYFRTPEINNSSGEMDGGGGSGGGGSGGG |
| | | GSAESHLSLLYHLTAVSSPAPGTPAFWVSGWLGPQQYLSYNSLRGEAEPCGAWVWE |
| | | NQVSWYWEKETTDLRIKEKLFLEAFKALGGKGPYTLQGLLGCELGPDNTSVPTAKFAL |
| | | NGEEFMNFDLKQGTWGGDWPEALAISQRWQQQDKAANKELTFLLFSCPHRLREHL |
| | | ERGRGNLEWKEPPSMRLKARPSSPGFSVLTCSAFSFYPPELQLRFLRNGLAAGTGQG |
| | | DFGPNSDGSFHASSSLTVKSGDEHHYCCIVQHAGLAQPLRVELESPAKSSGGGGSGG |
| | | GGSGGGGSEEELQVIQPDKSVLVAAGETATLRCTATSLIPVGPIQWFRGAGPGRELIY |
| | | NQKEGHFPRVTTVSDLTKRNNMDFSIRIGNITPADAGTYYCVKFRKGSPDDVEFKSG |
| | | AGTELSVRAKPSAPVVSGPAARATPQHTVSFTCESHGFSPRDITLKWFKNGNELSDF |
| | | QTNVDPVGESVSYSIHSTAKVVLTREDVHSQVICEVAHVTLQGDPLRGTANLSETIRV |
| | | PPTLEVTQQPVRAENQVNVTCQVRKFYPQRLQLTWLENGNVSRTETASTVTENKDG |
| | | TYNWMSWLLVNVSAHRDDVKLTCQVEHDGQPAVSKSHDLKVSAHPKEQGSNTAA |
| | | ENTGSNERNIYAAAHHHHHH |
| 21 | IL-7Rα- | ESGYAQNGDLEDAELDDYSFSCYSQLEVNGSQHSLTCAFEDPDVNITNLEFEICGALV |
| | Sirpα-hFcRn | EVKCLNFRKLQEIYFIETKKFLLIGKSNICVKVGEKSLTCKKIDLTTIVKPEAPFDLSVVYRE |
| | | GANDFVVTFNTSHLQKKYVKVLMHDVAYRQEKDENKWTHVNLSSTKLTLLQRKLQP |
| | | AAMYEIKVRSIPDHYFKGFWSEWSPSYYFRTPEINNSSGEMDGGGGSGGGGSGGG |
| | | GSEEELQVIQPDKSVLVAAGETATLRCTATSLIPVGPIQWFRGAGPGRELIYNQKEGH |
| | | FPRVTTVSDLTKRNNMDFSIRIGNITPADAGTYYCVKFRKGSPDDVEFKSGAGTELSV |
| | | RAKPSAPVVSGPAARATPQHTVSFTCESHGFSPRDITLKWFKNGNELSDFQTNVDPV |
| | | GESVSYSIHSTAKVVLTREDVHSQVICEVAHVTLQGDPLRGTANLSETIRVPPTLEVTQ |
| | | QPVRAENQVNVTCQVRKFYPQRLQLTWLENGNVSRTETASTVTENKDGTYNWMS |
| | | WLLVNVSAHRDDVKLTCQVEHDGQPAVSKSHDLKVSAHPKEQGSNTAAENTGSNE |
| | | RNIYGGGGGGGGSGGGGSAESHLSLLYHLTAVSSPAPGTPAFWVSGWLGPQQYLS |
| | | YNSLRGEAEPCGAWVWENQVSWYWEKETTDLRIKEKLFLEAFKALGGKGPYTLQGL |
| | | LGCELGPDNTSVPTAKFALNGEEFMNFDLKQGTWGGDWPEALAISQRWQQQDKA |
| | | ANKELTFLLFSCPHRLREHLERGRGNLEWKEPPSMRLKARPSSPGFSVLTCSAFSFYPP |
| | | ELQLRFLRNGLAAGTGQGDFGPNSDGSFHASSSLTVKSGDEHHYCCIVQHAGLAQPL |
| | | RVELESPAKSSAAAHHHHHH |
| 22 | hFcRn- | AESHLSLLYHLTAVSSPAPGTPAFWVSGWLGPQQYLSYNSLRGEAEPCGAWVWEN |
| | Sirpα-IL- | QVSWYWEKETTDLRIKEKLFLEAFKALGGKGPYTLQGLLGCELGPDNTSVPTAKFALN |
| | 7Rα | GEEFMNFDLKQGTWGGDWPEALAISQRWQQQDKAANKELTFLLFSCPHRLREHLE |
| | | RGRGNLEWKEPPSMRLKARPSSPGFSVLTCSAFSFYPPELQLRFLRNGLAAGTGQGD |
| | | FGPNSDGSFHASSSLTVKSGDEHHYCCIVQHAGLAQPLRVELESPAKSSGGGGSGGG |
| | | GSGGGGSEEELQVIQPDKSVLVAAGETATLRCTATSLIPVGPIQWFRGAGPGRELIYN |
| | | QKEGHFPRVTTVSDLTKRNNMDFSIRIGNITPADAGTYYCVKFRKGSPDDVEFKSGA |
| | | GTELSVRAKPSAPVVSGPAARATPQHTVSFTCESHGFSPRDITLKWFKNGNELSDFQT |
| | | NVDPVGESVSYSIHSTAKVVLTREDVHSQVICEVAHVTLQGDPLRGTANLSETIRVPPT |
| | | LEVTQQPVRAENQVNVTCQVRKFYPQRLQLTWLENGNVSRTETASTVTENKDGTYN |
| | | WMSWLLVNVSAHRDDVKLTCQVEHDGQPAVSKSHDLKVSAHPKEQGSNTAAENT |
| | | GSNERNIYGGGGSGGGGSGGGGSESGYAQNGDLEDAELDDYSFSCYSQLEVNGSQ |
| | | HSLTCAFEDPDVNITNLEFEICGALVEVKCLNFRKLQEIYFIETKKFLLIGKSNICVKVGEK |
| | | SLTCKKIDLTTIVKPEAPFDLSVVYREGANDFVVTFNTSHLQKKYVKVLMHDVAYRQE |
| | | KDENKWTHVNLSSTKLTLLQRKLQPAAMYEIKVRSIPDHYFKGFWSEWSPSYYFRTPE |
| | | INNSSGEMDAAAHHHHHH |
| 23 | hFcRn-IL- | AESHLSLLYHLTAVSSPAPGTPAFWVSGWLGPQQYLSYNSLRGEAEPCGAWVWEN |
| | 7Rα-Sirpα | QVSWYWEKETTDLRIKEKLFLEAFKALGGKGPYTLQGLLGCELGPDNTSVPTAKFALN |
| | | GEEFMNFDLKQGTWGGDWPEALAISQRWQQQDKAANKELTFLLFSCPHRLREHLE |
| | | RGRGNLEWKEPPSMRLKARPSSPGFSVLTCSAFSFYPPELQLRFLRNGLAAGTGQGD |
| | | FGPNSDGSFHASSSLTVKSGDEHHYCCIVQHAGLAQPLRVELESPAKSSGGGGSGGG |
| | | GSGGGGSESGYAQNGDLEDAELDDYSFSCYSQLEVNGSQHSLTCAFEDPDVNITNLE |
| | | FEICGALVEVKCLNFRKLQEIYFIETKKFLLIGKSNICVKVGEKSLTCKKIDLTTIVKPEAPF |
| | | DLSVVYREGANDFVVTFNTSHLQKKYVKVLMHDVAYRQEKDENKWTHVNLSSTKLT |
| | | LLQRKLQPAAMYEIKVRSIPDHYFKGFWSEWSPSYYFRTPEINNSSGEMDGGGGSGG |
| | | GGSGGGGSEEELQVIQPDKSVLVAAGETATLRCTATSLIPVGPIQWFRGAGPGRELIY |
| | | NQKEGHFPRVTTVSDLTKRNNMDFSIRIGNITPADAGTYYCVKFRKGSPDDVEFKSG |
| | | AGTELSVRAKPSAPVVSGPAARATPQHTVSFTCESHGFSPRDITLKWFKNGNELSDF |
| | | QTNVDPVGESVSYSIHSTAKVVLTREDVHSQVICEVAHVTLQGDPLRGTANLSETIRV |
| | | PPTLEVTQQPVRAENQVNVTCQVRKFYPQRLQLTWLENGNVSRTETASTVTENKDG |
| | | TYNWMSWLLVNVSAHRDDVKLTCQVEHDGQPAVSKSHDLKVSAHPKEQGSNTAA |
| | | ENTGSNERNIYAAAHHHHHH |
| 24 | B2m-IL- | IQRTPKIQVYSRHPAENGKSNFLNCYVSGFHPSDIEVDLLKNGERIEKVEHSDLSFSKD |
| | 7Rα-Sirpα | WSFYLLYYTEFTPTEKDEYACRVNHVTLSQPKIVKWDRDMGGGGSGGGGSGGGGS |
| | | ESGYAQNGDLEDAELDDYSFSCYSQLEVNGSQHSLTCAFEDPDVNITNLEFEICGALV |
| | | EVKCLNFRKLQEIYFIETKKFLLIGKSNICVKVGEKSLTCKKIDLTTIVKPEAPFDLSVVYRE |
| | | GANDFVVTFNTSHLQKKYVKVLMHDVAYRQEKDENKWTHVNLSSTKLTLLQRKLQP |
| | | AAMYEIKVRSIPDHYFKGFWSEWSPSYYFRTPEINNSSGEMDGGGGSGGGGSGGG |
| | | GSEEELQVIQPDKSVLVAAGETATLRCTATSLIPVGPIQWFRGAGPGRELIYNQKEGH |
| | | FPRVTTVSDLTKRNNMDFSIRIGNITPADAGTYYCVKFRKGSPDDVEFKSGAGTELSV |
| | | RAKPSAPVVSGPAARATPQHTVSFTCESHGFSPRDITLKWFKNGNELSDFQTNVDPV |
| | | GESVSYSIHSTAKVVLTREDVHSQVICEVAHVTLQGDPLRGTANLSETIRVPPTLEVTQ |
| | | QPVRAENQVNVTCQVRKFYPQRLQLTWLENGNVSRTETASTVTENKDGTYNWMS |
| | | WLLVNVSAHRDDVKLTCQVEHDGQPAVSKSHDLKVSAHPKEQGSNTAAENTGSNE |
| | | RNIY |
| 25 | B2m-Sirpα | IQRTPKIQVYSRHPAENGKSNFLNCYVSGFHPSDIEVDLLKNGERIEKVEHSDLSFSKD |
| | | WSFYLLYYTEFTPTEKDEYACRVNHVTLSQPKIVKWDRDMGGGGSGGGGSGGGGS |
| | | EEELQVIQPDKSVLVAAGETATLRCTATSLIPVGPIQWFRGAGPGRELIYNQKEGHFP |
| | | RVTTVSDLTKRNNMDFSIRIGNITPADAGTYYCVKFRKGSPDDVEFKSGAGTELSVRA |
| | | KPSAPVVSGPAARATPQHTVSFTCESHGFSPRDITLKWFKNGNELSDFQTNVDPVGE |
| | | SVSYSIHSTAKVVLTREDVHSQVICEVAHVTLQGDPLRGTANLSETIRVPPTLEVTQQP |
| | | VRAENQVNVTCQVRKFYPQRLQLTWLENGNVSRTETASTVTENKDGTYNWMSWL |
| | | LVNVSAHRDDVKLTCQVEHDGQPAVSKSHDLKVSAHPKEQGSNTAAENTGSNERNI |
| | | Y |
| 26 | B2m-hFcRn- | IQRTPKIQVYSRHPAENGKSNFLNCYVSGFHPSDIEVDLLKNGERIEKVEHSDLSFSKD |
| | IL-7Rα- | WSFYLLYYTEFTPTEKDEYACRVNHVTLSQPKIVKWDRDMGGGGSGGGGSGGGGS |
| | Sirpα | AESHLSLLYHLTAVSSPAPGTPAFWVSGWLGPQQYLSYNSLRGEAEPCGAWVWEN |
| | | QVSWYWEKETTDLRIKEKLFLEAFKALGGKGPYTLQGLLGCELGPDNTSVPTAKFALN |
| | | GEEFMNFDLKQGTWGGDWPEALAISQRWQQQDKAANKELTFLLFSCPHRLREHLE |
| | | RGRGNLEWKEPPSMRLKARPSSPGFSVLTCSAFSFYPPELQLRFLRNGLAAGTGQGD |
| | | FGPNSDGSFHASSSLTVKSGDEHHYCCIVQHAGLAQPLRVELESPAKSSGGGGSGGG |
| | | GSGGGGSESGYAQNGDLEDAELDDYSFSCYSQLEVNGSQHSLTCAFEDPDVNITNLE |
| | | FEICGALVEVKCLNFRKLQEIYFIETKKFLLIGKSNICVKVGEKSLTCKKIDLTTIVKPEAPF |
| | | DLSVVYREGANDFVVTFNTSHLQKKYVKVLMHDVAYRQEKDENKWTHVNLSSTKLT |
| | | LLQRKLQPAAMYEIKVRSIPDHYFKGFWSEWSPSYYFRTPEINNSSGEMDGGGGSGG |
| | | GGSGGGGSEEELQVIQPDKSVLVAAGETATLRCTATSLIPVGPIQWFRGAGPGRELIY |
| | | NQKEGHFPRVTTVSDLTKRNNMDFSIRIGNITPADAGTYYCVKFRKGSPDDVEFKSG |
| | | AGTELSVRAKPSAPVVSGPAARATPQHTVSFTCESHGFSPRDITLKWFKNGNELSDF |
| | | QTNVDPVGESVSYSIHSTAKVVLTREDVHSQVICEVAHVTLQGDPLRGTANLSETIRV |
| | | PPTLEVTQQPVRAENQVNVTCQVRKFYPQRLQLTWLENGNVSRTETASTVTENKDG |
| | | TYNWMSWLLVNVSAHRDDVKLTCQVEHDGQPAVSKSHDLKVSAHPKEQGSNTAA |
| | | ENTGSNERNIYAAAHHHHHH |
| 27 | B2m-hFcRn- | IQRTPKIQVYSRHPAENGKSNFLNCYVSGFHPSDIEVDLLKNGERIEKVEHSDLSFSKD |
| | Sirpα-IL- | WSFYLLYYTEFTPTEKDEYACRVNHVTLSQPKIVKWDRDMGGGGSGGGGSGGGGS |
| | 7Rα | AESHLSLLYHLTAVSSPAPGTPAFWVSGWLGPQQYLSYNSLRGEAEPCGAWVWEN |
| | | QVSWYWEKETTDLRIKEKLFLEAFKALGGKGPYTLQGLLGCELGPDNTSVPTAKFALN |
| | | GEEFMNFDLKQGTWGGDWPEALAISQRWQQQDKAANKELTFLLFSCPHRLREHLE |
| | | RGRGNLEWKEPPSMRLKARPSSPGFSVLTCSAFSFYPPELQLRFLRNGLAAGTGQGD |
| | | FGPNSDGSFHASSSLTVKSGDEHHYCCIVQHAGLAQPLRVELESPAKSSGGGGSGGG |
| | | GSGGGGSEEELQVIQPDKSVLVAAGETATLRCTATSLIPVGPIQWFRGAGPGRELIYN |
| | | QKEGHFPRVTTVSDLTKRNNMDFSIRIGNITPADAGTYYCVKFRKGSPDDVEFKSGA |
| | | GTELSVRAKPSAPVVSGPAARATPQHTVSFTCESHGFSPRDITLKWFKNGNELSDFQT |
| | | NVDPVGESVSYSIHSTAKVVLTREDVHSQVICEVAHVTLQGDPLRGTANLSETIRVPPT |
| | | LEVTQQPVRAENQVNVTCQVRKFYPQRLQLTWLENGNVSRTETASTVTENKDGTYN |
| | | WMSWLLVNVSAHRDDVKLTCQVEHDGQPAVSKSHDLKVSAHPKEQGSNTAAENT |
| | | GSNERNIYGGGGSGGGGSGGGGSESGYAQNGDLEDAELDDYSFSCYSQLEVNGSQ |
| | | HSLTCAFEDPDVNITNLEFEICGALVEVKCLNFRKLQEIYFIETKKFLLIGKSNICVKVGEK |
| | | SLTCKKIDLTTIVKPEAPFDLSVVYREGANDFVVTFNTSHLQKKYVKVLMHDVAYRQE |
| | | KDENKWTHVNLSSTKLTLLQRKLQPAAMYEIKVRSIPDHYFKGFWSEWSPSYYFRTPE |
| | | INNSSGEMDAAAHHHHHH |
| 28 | B2m-IL- | IQRTPKIQVYSRHPAENGKSNFLNCYVSGFHPSDIEVDLLKNGERIEKVEHSDLSFSKD |
| | 7Rα-hFcRn- | WSFYLLYYTEFTPTEKDEYACRVNHVTLSQPKIVKWDRDMGGGGSGGGGSGGGGS |
| | Sirpα | ESGYAQNGDLEDAELDDYSFSCYSQLEVNGSQHSLTCAFEDPDVNITNLEFEICGALV |
| | | EVKCLNFRKLQEIYFIETKKFLLIGKSNICVKVGEKSLTCKKIDLTTIVKPEAPFDLSVVYRE |
| | | GANDFVVTFNTSHLQKKYVKVLMHDVAYRQEKDENKWTHVNLSSTKLTLLQRKLQP |
| | | AAMYEIKVRSIPDHYFKGFWSEWSPSYYFRTPEINNSSGEMDGGGGSGGGGSGGG |
| | | GSAESHLSLLYHLTAVSSPAPGTPAFWVSGWLGPQQYLSYNSLRGEAEPCGAWVWE |
| | | NQVSWYWEKETTDLRIKEKLFLEAFKALGGKGPYTLQGLLGCELGPDNTSVPTAKFAL |
| | | NGEEFMNFDLKQGTWGGDWPEALAISQRWQQQDKAANKELTFLLFSCPHRLREHL |
| | | ERGRGNLEWKEPPSMRLKARPSSPGFSVLTCSAFSFYPPELQLRFLRNGLAAGTGQG |
| | | DFGPNSDGSFHASSSLTVKSGDEHHYCCIVQHAGLAQPLRVELESPAKSSGGGGSGG |
| | | GGSGGGGSEEELQVIQPDKSVLVAAGETATLRCTATSLIPVGPIQWFRGAGPGRELIY |
| | | NQKEGHFPRVTTVSDLTKRNNMDFSIRIGNITPADAGTYYCVKFRKGSPDDVEFKSG |
| | | AGTELSVRAKPSAPVVSGPAARATPQHTVSFTCESHGFSPRDITLKWFKNGNELSDF |
| | | QTNVDPVGESVSYSIHSTAKVVLTREDVHSQVICEVAHVTLQGDPLRGTANLSETIRV |
| | | PPTLEVTQQPVRAENQVNVTCQVRKFYPQRLQLTWLENGNVSRTETASTVTENKDG |
| | | TYNWMSWLLVNVSAHRDDVKLTCQVEHDGQPAVSKSHDLKVSAHPKEQGSNTAA |
| | | ENTGSNERNIYAAAHHHHHH |
| 29 | B2m- | IQKTPQIQVYSRHPPENGKPNILNCYVTQFHPPHIEIQMLKNGKKIPKVEMSDMSFSK |
| | msFcRn- | DWSFYILAHTEFTPTETDTYACRVKHASMAEPKTVYWDRDMGGGGSGGGGSGGG |
| | hDSG3 | GSSETRPPLMYHLTAVSNPSTGLPSFWATGWLGPQQYLTYNSLRQEADPCGAWM |
| | | WENQVSWYWEKETTDLKSKEQLFLEALKTLEKILNGTYTLQGLLGCELASDNSSVPTA |
| | | VFALNGEEFMKFNPRIGNWTGEWPETEIVANLWMKQPDAARKESEFLLNSCPERLL |
| | | GHLERGRRNLEWKEPPSMRLKARPGNSGSSVLTCAAFSFYPPELKFRFLRNGLASGSG |
| | | NCSTGPNGDGSFHAWSLLEVKRGDEHHYQCQVEHEGLAQPLTVDLDSSARSSGGG |
| | | GSGGGGSGGGGSGSEWVKFAKPCREGEDNSKRNPIAKITSDYQATQKITYRISGVGI |
| | | DQPPFGIFVVDKNTGDINITAIVDREETPSFLITCRALNAQGLDVEKPLILTVKILDINDN |
| | | PPVFSQQIFMGEIEENSASNSLVMILNATDADEPNHLNSKIAFKIVSQEPAGTPMFLLS |
| | | RNTGEVRTLTNSLDREQASSYRLVVSGADKDGEGLSTQCECNIKVKDVNDNFPMFR |
| | | DSQYSARIEENILSSELLRFQVTDLDEEYTDNWLAVYFFTSGNEGNWFEIQTDPRTNE |
| | | GILKVVKALDYEQLQSVKLSIAVKNKAEFHQSVISRYRVQSTPVTIQVINVREGIAFRPA |
| | | SKTFTVQKGISSKKLVDYILGTYQAIDEDTNKAASNVKYVMGRNDGGYLMIDSKTAEI |
| | | KFVKNMNRDSTFIVNKTITAEVLAIDEYTGKTSTGTVYVRVPDFNDNCPTAVLEKDAV |
| | | CSSSPSVVVSARTLNNRYTGPYTFALEDQPVKLPAVWSITTLNATSALLRAQEQIPPGV |
| | | YHISLVLTDSQNNRCEMPRSLTLEVCQCDNRGICGTSYPTTSPGTRYGRPHSGRAAAH |
| | | HHHHH |

Gene for the different constructs were synthetised and cloned downstream the IL-2 leader sequence in pCDNA3.4 vector. Recombinant constructs were transiently expressed in FreeStyle™ 293-F Cells (Life Technologies) at 37° C., 8% CO₂ with shaking, following transfection with FectoPRO and FectoPRO Booster (Polyplus).

Seven days post-transfection, the His-tag containing molecules were purified from culture supernatant by IMAC using a HisTrap excel 5 mL column (Cytivia). Briefly, the column was equilibrated for five column volumes with PBS, 500 mM NaCl pH7.4, the cell supernatant, that was previously filtered (0.2 um), was loaded onto the column which was then washed with PBS+500 mM NaCl, 10 mM Imidazole pH7.4 for ten column volumes. The His-tag containing molecules were eluted from the column with PBS, 500 mM NaCl, 500 mM Imidazole pH7.4 for ten column volumes.

The Fc-containing molecules were purified using a MabSelect 5 mL colum (Cytivia). Briefly, the column was equilibrated with PBS for five column volumes, the cell supernatant, that was previously filtered (0.2 um), was loaded onto the column which was then washed with PBS for 10 column volumes. The FcRn molecules were eluted from the column with 100 mM citric acid pH3; the proteins were immediately neutralised with 1M Tris pH9.

All the molecules were further purified by size-exclusion chromatography in PBS using a Hi-load 16/600 column Superdex 200 μg column (Cytivia).

### Characterisation of Binding on BLitz

Ni-NTA biosensors (Fortebio #18-5102) were hydrated in 1× Kinectics Buffer for 10 mins prior to the experiment. The biosensors were first dipped for 30s in 1× Kinetics Buffer to establish a baseline then the FcRn molecules (at 20 μg/mL in 1× Kinetics Buffer) were captured for 180s onto the NiNTA biosensor via their HisTag. A second baseline was established before the association by dipping the biosensor for 30s in PBS at the same pH than the association step. Human poly IgGs or mouse anti-hSIRPα antibody (at 20 μg/mL in PBS at the desired pH) were let to associate with the FcRn molecules by dipping the biosensor in the antibody solution for 120s then was let to dissociate by dipping the biosensor in the same buffer solution than the second baseline. A step correction at the beginning of the association and the dissociation were applied for the fitting.

### Pharmacokinetics in Mice (FIG. 2-10)

6/7 weeks old Balb/c mice were injected with FcRn molecules alone, or anti SIRPα antibody one day before and then, FcRn molecules and either anti SIRPα antibody+anti IL7Rα antibody one day before and then, FcRn molecules.

Intraperitoneal injections were realized for each injection. Incision at the tail of the mouse was realized to recover 4 μL per time point and centrifuged at 2500 t/min during 10 min and stocked at −20° C.

### Pharmacokinetics in NHP:

Two Macaca fascicularis (3.6 Kg and 4.1 Kg) were injected with anti-IL-7Rα antibody (Baccinex F19262) and anti-SIRPα (Lcp21-cp13) at 1 mg/kg by intravenous way during 15 min at day −2. At Day 0, β2m-hFcRn-SIRPα-004 were injected at 10 mg/kg by intravenous way during 15 min. Temperature, Saturation of 02, Cardiac frequency and PAM of NHP were monitored. IgG, IgA, IgM, prealbumin, albumin and fibrinogen were assessed by a platform at Nantes University Hospital Center. (FIG. 11/12/13)

Two Macaca fascicularis (3.6 Kg and 4.1 Kg) were injected with anti-IL-7Rα antibody (Baccinex F17221) and anti-SIRPα (22 Jun. 2017) at 1 mg/kg by intravenous way during 15 min at day −2. At Day 0.1 and 2 β2m-hFcRn-SIRPα-004 were injected at 10 mg/kg by intravenous way during 15 min. Temperature, Saturation of 02, Cardiac frequency and PAM of NHP were monitored. IgG, IgA, IgM, prealbumin, albumin and fibrinogen were assessed by a platform at Nantes University Hospital Center. (FIG. 15/16/17)

Immunized Mice Model with Peptide to Induce Humoral B Cell Response (FIG. 14)

6/7 weeks old Balb/c mice were immunized with emulsion containing two peptides designed to induce B cell humoral response (from RBD viral protein) and montanide described to increase immune response at day 0 and 7.

Antigen preparation: The peptide antigens, aKXVAAWTLKAAaNSNNLDSKVGGNYNYLYRLFRKS (SEQ ID NO: 19): pB1 and aKXVAAWTLKAAaNYNYLYRLFRKSNLKPFERDISTEIYQA (SEQ ID NO: 20): pB4 were purchased from Synpeptide Co., Ltd as lyophilized powders, with a indicating a d-alanine and X a cyclohexylalanine. The peptides were reconstituted in DMSO to (Sigma, D8418-250ML) a concentration of 50 mg/ml and intermediate concentration to prepare emulsion in PBS (phosphate buffered saline) at 2.2 mg/ml.

Making the emulsion: The peptide-montanide emulsions were prepared by mixing together the peptide solution with the montanide suspension at a ratio of 0.9:1.1, using two glass syringes, one loaded with the adjuvant, and the other with the antigen solution in PBS, connecting them with a 3 way stop cock. Care was taken to first introduce the peptide solution slowly into the montanide suspension drop by drop before mixing thoroughly. The protein-montanide emulsion was tested for readiness by putting a drop of emulsion onto PBS.

Each mouse was injected subcutaneously in the left footpad the first week and in the right footpad the second week with 50 μl of the montanide emulsion containing 50 μg of each peptide.

Mice were intraperitoneally injected with 100 μl of PBS or FcRn molecules (β2m-mFcRn-vRBD-004) or ARGX113 (Fc mutated molecule). Mice were forced-fed with Mycophenolate mofetil at different time points at 50 mg/kg.

Incision at the tail of the mouse was realized to recover 4 μL per time point and centrifuged at 2500 t/min during 10 min and stocked at −20° C.

### Pharmacokinetics in Mice In Vivo of FcRn Molecules Alone (FIG. 2)

Drug concentration in the plasma was determined by ELISA using an anti-SIRPα antibody (LCP2/Cp14 (12/05/17)) immobilized on plastic at 1 μg/mL in borate buffer (pH 9), purified FcRn molecules were added at 1 μg/mL for the first point and diluted up 3 to 3. After incubation and washing, biotinylated mouse anti-HIS (MBL #D291-6) and peroxidase-labeled streptavidin (Jackson immunoresearch; USA; reference 016-030-084) were added during one hour and revealed by conventional methods.

### Pharmacokinetics in Mice In Vivo of FcRn Molecules in Presence of Anti-IL7Rα and Anti-SIRPα Antibodies (FIG. 10)

Drug concentration in the plasma was determined by ELISA using, anti-β2m (invitrogen #PA5-80367) immobilized on plastic at 1 μg/mL in borate buffer (pH 9), purified FcRn molecules were added at 1 μg/mL for the first point and diluted up 4 to 4. After incubation and washing, biotinylated mouse anti-HIS (MBL #D291-6,006/13052520/OG-HIS) and peroxidase-labeled streptavidin (Jackson immunoresearch; USA; reference 016-030-084) were added during one hour and revealed by conventional methods.

### Pharmacokinetics in Mice In Vivo of Anti-SIRPα Antibody in Presence of FcRn Molecules (FIGS. 3, 5 and 6)

Anti-SIRPα antibody concentration in the plasma was determined by ELISA using mouse anti-human kappa antibody (#1.02 mg/ml, 18 Apr. 2018) immobilized on plastic at 1 μg/mL in borate buffer (pH 9), purified anti-SIRPα antibody (LCP2/Cp14 (12 May 2017))) were added at 1 μg/mL for the first point and diluted up 4 to 4. After incubation and washing, donkey anti-human HRP (#709-035-149, Jackson Laboratory) was added during one hour and revealed by conventional methods.

### Pharmacokinetics in Mice In Vivo of Anti-SIRPα Antibody in Presence of FcRn Molecules and Anti-IL7Rα Antibody (FIGS. 4, 7 and 8)

Anti-SIRPα antibody concentration in the plasma was determined by ELISA using anti-idiotype SIRPα antibody (nb L650.18014.1) immobilized on plastic at 2 μg/mL in borate buffer (pH 9), purified anti-SIRPα antibody (LCP2/Cp14 (12 May 2017)) were added at 1 μg/mL for the first point and diluted up 4 to 4. After incubation and washing, donkey anti-human HRP (#709-035-149, Jackson Laboratory) was added during one hour and revealed by conventional methods.

### Pharmacokinetics in Mice In Vivo of Anti-IL7Rα Antibody in Presence of FcRn Molecules and Anti-SIRPα Antibody (FIGS. 4 and 7)

Anti-IL7Rα antibody concentration in the plasma was determined by ELISA using anti-idiotype IL7Rα antibody (nb L650.18013.1) immobilized on plastic at 1 μg/mL in borate buffer (pH 9), purified anti-IL7Rα antibody (Baccinex lot F17221) were added at 1 μg/mL for the first point and diluted up 4 to 4. After incubation and washing, donkey anti-human HRP (#709-035-149, Jackson Laboratory) was added during one hour and revealed by conventional methods.

### Pharmacokinetics in Mice In Vivo of Anti-IL7Rα Antibody in Presence of FcRn Molecules and Anti-SIRPα Antibody (FIG. 8)

Anti-IL7Rα antibody concentration in the plasma was determined by ELISA using CD127-Fc (CD127-fc; 306-IR) immobilized on plastic at 1 μg/mL in carbonate buffer (pH 9), purified anti-IL7Rα antibody (Baccinex lot F17221) were added at 1 μg/mL for the first point and diluted up 4 to 4. After incubation and washing, mouse anti-human kappa antibody (#1.02 mg:ml, 18/04/18) was added during one hour. After incubation and washing, donkey Anti-MsPO (JI #715-036-151, lot 104986) was added during one hour and revealed by conventional methods.

### Concentration of Albumin in the Sera of Mice (FIG. 9)

Concentration of albumin in the plasma was determined by Mouse Albumin Matched Antibody Pair Kit (ab210890/GR3339694-11/Q6923) using Capture Ab (3500021/Q6924) immobilized on plastic at 2 μg/mL in carbonate buffer (pH 9), Albumin (3200148/Q6926) were added at 8 μg/mL for the first point and diluted up 2 to 2. After incubation and washing, Detection Ab (350022/Q6925) was added during one hour. After incubation and washing, Peroxidase-labeled streptavidin (Jackson Immunoresearch; USA; reference 016-030-084) was added during one hour and revealed by conventional methods.

### Titer of Anti-vRBD Antibodies in the Sera of Immunized Mice Model (FIG. 14)

Titer of anti-vRBD antibodies in the plasma was determined by ELISA using RBD protein labeled His (SinoBio #40592-V08B) immobilized on plastic at 2 μg/mL in carbonate buffer (pH 9). After incubation and washing, detection donkey anti-mouse IgG labelled peroxidase (Jackson Immunoresearch #715-036-151) was added during one hour and revealed by conventional methods.

### Pharmacokinetics in NHP In Vivo of Anti-SIRPα Antibody in Presence of FcRn Molecules and Anti-IL7Rα Antibody (FIGS. 11 and 15)

Anti-SIRPα antibody concentration in the plasma was determined by ELISA using anti-idiotype antibody (nb L650.18014.1) immobilized on plastic at 2 μg/mL in borate buffer (pH 9), purified anti-SIRPα antibody (LCP2/Cp14 (12 May 2017): FIG. 10 or 22/06/17: FIG. 14) were added at 1 μg/mL for the first point and diluted up 4 to 4. After incubation and washing, mouse anti-human kappa antibody (#1.02 mg:ml, 18 Apr. 2018) was added during one hour. Donkey anti-mouse PO (JI #715-036-151, lot 104986) was added during one hour and revealed by conventional methods.

### Pharmacokinetics in NHP In Vivo of Anti-IL7Rα Antibody in Presence of FcRn Molecules and Anti-SIRPα Antibody (FIGS. 11 and 15)

Anti-IL7Rα antibody concentration in the plasma was determined by ELISA using CD127-Fc (CD127-fc; 306-IR) immobilized on plastic at 1 μg/mL in carbonate buffer (pH 9), purified anti-IL7Rα antibody ((Baccinex lot F17221): FIG. 10 or (Baccinex lot F17221): FIG. 14) were added at 1 μg/mL for the first point and diluted up 4 to 4. After incubation and washing, mouse anti-human kappa antibody (#1.02 mg:ml, 18 Apr. 2018) was added during one hour. After incubation and washing, Donkey anti-mouse PO (JI #715-036-151, lot 104986) was added during one hour and revealed by conventional methods.

### Pharmacokinetics in Mice In Vivo of Anti-SIRPα Antibody in Presence of Bispecific FcRn Molecule and Anti-IL7Rα Antibody (FIG. 18)

Anti-SIRPα antibody concentration in the plasma was determined by ELISA using anti-idiotype SIRPα antibody (nb L650.18014.1) immobilized on plastic at 2 μg/mL in borate buffer (pH 9). Purified anti-SIRPα antibody (LCP2/Cp14 (12 May 2017)) was used as standard at 1 μg/mL for the first point and diluted following a 4-fold serial dilution. After incubation and washing, donkey anti-human HRP (#709-035-149, Jackson Laboratory) was added during one hour and revealed by conventional methods.

### Pharmacokinetics in Mice In Vivo of Anti-IL7Rα Antibody in Presence of Bispecific FcRn Molecule and Anti-SIRPα Antibody (FIG. 18)

Anti-IL7Rα antibody concentration in the plasma was determined by ELISA using CD127-Fc (CD127-fc; 306-IR) immobilized on plastic at 1 μg/mL in carbonate buffer (pH 9). Purified anti-IL7Rα antibody (Baccinex lot F17221) was used as standard at 1 μg/mL for the first point and diluted following a 4-fold serial dilution. After incubation and washing, mouse anti-human kappa antibody (#1.02 mg:ml, 18/04/18) was added during one hour. After incubation and washing, donkey Anti-MsPO (JI #715-036-151, lot 104986) was added during one hour and revealed by conventional methods.

### Pharmacokinetics in Mice In Vivo of Anti-IL7Rα Antibody in Presence of FcRn Molecules and Anti-SIRPα Antibody (FIGS. 20, 21 and 24)

Anti-IL7Rα antibody concentration in the plasma was determined by ELISA using CD127-Fc (CD127-fc; 306-IR) immobilized on plastic at 1 μg/mL in carbonate buffer (pH 9), purified humanized anti-IL7Rα antibody were added at 1 μg/mL for the first point and diluted up 4 to 4 or 3 to 3. After incubation and washing, mouse anti-human kappa antibody (#0.78 mg/ml, 23/04/16) was added during one hour. After incubation and washing, donkey Anti-MsPO (JI #715-036-151, lot 160248) was added during one hour and revealed by conventional methods.

### Pharmacokinetics in Mice In Vivo of Anti-SIRPα Antibody in Presence of FcRn Molecules and Anti-IL7Rα Antibody (FIGS. 20, 21 and 24)

Anti-SIRPα antibody concentration in the plasma was determined by ELISA using anti-idiotype SIRPα antibody (nb L650.18014.1) immobilized on plastic at 2 μg/mL in borate buffer (pH 9), humanized anti-SIRPα IgG4 mutated (S228P) antibody or humanized anti-SIRPα IgG1 mutated (E333A) were added at 1 μg/mL for the first point and diluted up 4 to 4. After incubation and washing, donkey anti-human HRP (#709-035-149, lot 153143, Jackson Laboratory) was added during one hour and revealed by conventional methods.

### Pharmacokinetics in Mice In Vivo of Anti-SIRPα/γ Antibody in Presence of FcRn Molecules and Anti-IL7Rα Antibody (FIG. 24)

Anti-SIRPα antibody concentration in the plasma was determined by ELISA using SIRPy (11828-H08H, Sinobiolog) immobilized on plastic at 2 μg/mL in carbonate buffer (pH 9), human anti-SIRPα/γ IgG4 mutated (S228P) were added at 1 μg/mL for the first point and diluted up 4 to 4. After incubation and washing, donkey anti-human HRP (#709-035-149, lot 153143, Jackson Laboratory) was added during one hour and revealed by conventional methods.

Immunized Mice Model with vRBD to Induce Humoral B Cell Response (FIG. 22)

6/7 weeks old Balb/c mice were immunized with emulsion containing vRBD and CFA describe to increase immune response at day 0.

Antigen preparation: The vRBD protein was purchased from Sinobiological. The protein was reconstituted in PBS to a concentration of 250 μg/ml and intermediate concentration to prepare emulsion in PBS (phosphate buffered saline) at 40 μg/ml.

Making the emulsion: The protein-CFA emulsions were prepared by mixing together the protein solution with the CFA suspension at a ratio of 1/1, using two glass syringes, one loaded with the adjuvant, and the other with the antigen solution in PBS, connecting them with a 3 way stop cock. Care was taken to first introduce the peptide solution slowly into the CFA suspension drop by drop before mixing thoroughly. The protein-CFA emulsion was tested for readiness by putting a drop of emulsion onto PBS.

Each mouse was injected subcutaneously in the left footpad the first week with 50 μl of the CFA emulsion containing 1 μg of protein.

Sera was isolated after an intracardiac. Sera was recovered after a centrifugation at 2500 t/min during 10 min and intraperitoneally injected in balb/c WT mice. Mice were injected with 100 μl of sera containing 149 μg of mouse anti vRBD antibodies or mice were injected with 100 μl of sera containing 29.8 μg of mouse anti vRBD antibodies and mice were injected with 100 μl of sera containing 5.96 μg of mouse anti vRBD antibodies.

β2-msFcRn-vRBD molecules was injected at day 1 and 2 at 20 mg/kg.

Incision at the tail of the mouse was realized to recover 4 μL per time point and centrifuged at 2500 t/min during 10 min and stocked at −20° C.

Immunized Mice Model with hDSG3 to Induce Humoral B Cell Response (FIG. 23)

6/7 weeks old Balb/c mice were immunized with emulsion containing hDSG3 and CFA describe to increase immune response at day 0, 7, 14, 21.

Antigen preparation: The hDSG3 protein was purchased from R&D System. The protein was reconstituted in PBS to a concentration of 250 μg/ml and intermediate concentration to prepare emulsion in PBS (phosphate buffered saline) at 10 μg/ml.

Making the emulsion: The protein-CFA emulsions were prepared by mixing together the protein solution with the CFA suspension at a ratio of 1/1, using two glass syringes, one loaded with the adjuvant, and the other with the antigen solution in PBS, connecting them with a 3 way stop cock. Care was taken to first introduce the peptide solution slowly into the CFA suspension drop by drop before mixing thoroughly. The protein-CFA emulsion was tested for readiness by putting a drop of emulsion onto PBS.

Each mouse was injected subcutaneously in the left footpad the first week with 50 μl of the CFA emulsion containing 250 ng of protein.

Sera was isolated after an intracardiac. Sera was recovered after a centrifugation at 2500 t/min during 10 min and intraperitoneally injected in balb/c WT mice. Mice were injected with 200 ul of sera containing mouse anti-hDSG3 antibodies. β2-msFcRn-hDSG3 molecules was injected at day 1 at 40 mg/kg.

Incision at the tail of the mouse was realized to recover 4 μL per time point and centrifuged at 2500 t/min during 10 min and stocked at −20° C.

1-17. (canceled)

18. A molecule for selective clearance of an antibody directed against an antigen, wherein the molecule comprises
an extracellular part of a human neonatal Fc receptor (FcRn) including regions alpha1;
alpha2 and alpha3 and devoid of transmembrane domain; and
a beta-2 microglobulin;
said extracellular part of FcRn and/or said beta-2 microglobulin being covalently linked to the antigen of the antibody to be depleted or a fragment of said antigen which can be bound by the antibody to be depleted.

19. The molecule of claim 18, wherein the molecule comprises a single polypeptide chain comprising the extracellular part of FcRn, the beta-2 microglobulin and the antigen or the fragment thereof.

20. The molecule of claim 19, wherein the molecule comprises, from the N terminus to the C terminus, the beta-2 microglobulin, the region alpha1, the region alpha2, the region alpha3 and the antigen or the fragment thereof.

21. The molecule of claim 18, wherein the molecule comprises two polypeptide chains, a first polypeptide chain comprising the extracellular part of FcRn and a second polypeptide chain comprising the beta-2 microglobulin, and the antigen or the fragment thereof is covalently linked to the first polypeptide chain, the second polypeptide chain or both.

22. The molecule of claim 21, wherein the first polypeptide chain comprises, from the N terminus to the C terminus, the antigen or the fragment thereof, the region alpha1, the region alpha2 and the region alpha3; or the region alpha1, the region alpha2, the region alpha3 and the antigen or the fragment thereof.

23. The molecule of claim 21, wherein the second polypeptide chain comprises, from the N terminus to the C terminus, the antigen or the fragment thereof and the beta-2 microglobulin; or the beta-2 microglobulin and the antigen or the fragment thereof.

24. The molecule of claim 18, wherein the molecule comprises several antigens or fragments thereof, the antigens being identical or different.

25. The molecule of claim 18, wherein the molecule binds human fragment crystallizable region (Fc region) of the antibody at endosomal pH or a pH from 5.5 to 6.5 but not at blood physiological pH.

26. The molecule of claim 18, wherein the antibody binds the antigen or a fragment thereof of the molecule at blood physiological pH and at endosomal pH.

27. The molecule of claim 18, wherein the antigen is selected from the group consisting of 60 kDa SS-A/Ro ribonucleoprotein, antigen La, a double-stranded DNA, histone, snRNP core protein, glycoprotein IIb, glycoprotein IIIa, glycoprotein Ib, glycoprotein IX, neurofascin 155, contactin 1, Topoisomerase I, centromere, histidine-tRNA ligase, sp100 nuclear antigen, nucleoporin 210 kDa, actin, cyclic citrullinated peptide, myeloperoxidase, proteinase 3, cardiolipin, carbamylated protein, phospholipid, collagen, thrombin, nicotinic acetylcholine receptor, muscle-specific kinase, voltage-gated calcium channel (P/Q-type), vinculin, thyroid peroxidase, thyroglobulin, thyrotropin receptor, neuronal nuclear protein, glutamate receptor, amphiphysin, glutamate decarboxylase, voltage-gated potassium channel, collapsin response mediator protein 5, N-methyl-D-aspartate receptor, aquaporin-4, desmoglein 3, desmoglein 1, phospholipase A2 receptor, myelin oligodendrocyte glycoprotein (MOG), myelin basic protein, proteolipid protein, myelin-associated glycoprotein, myelin-associated oligodendrocyte basic protein, transaldolase, low density lipoprotein receptor related protein 4, insulin, islet antigen 2, glutamic acid decarboxylase 65, zinc transporter 8, cartilage gp39, gp130-RAPS, 65 kDa heat shock protein, fibrillarin, small nuclear protein (snoRNP), thyroid stimulating factor receptor, nuclear antigens, glycoprotein gp70, ribosomes, pyruvate dehydrogenase dehydrolioamide acetyltransferase, hair follicle antigens, human tropomyosin isoform 5, a-amino-3-hydroxy-5-methyl-4-isoxazolepropionic acid (AMP A) receptor, GABAA and GABAB receptors, glycine receptor, and dipeptidyl-peptidase-like protein 6 (DPPX).

28. The molecule of claim 18, wherein the extracellular part of FcRn is modified for preventing or reducing the binding to albumin and/or fibrinogen.

29. The molecule of claim 18, wherein the extracellular part of FcRn comprises a mutation of one or several of the amino acids selected from the group consisting of W51, W53, W59, W61 and H166 corresponding to the amino acid positions as shown in SEQ ID NO: 2.

30. The molecule of claim 18, wherein the extracellular part of FcRn comprises a mutation of one or several of the amino acids selected from the group consisting of W51A, W53A, W59A, W61A and H166A corresponding to the amino acid positions as shown in SEQ ID NO: 2.

31. A pharmaceutical composition comprising a molecule according to claim 18 or a nucleic acid or set of nucleic acids encoding said molecule.

32. A method for treating an autoimmune disease, an inflammatory disease or disorder, or a transplant rejection in a subject in need thereof comprising administering a therapeutically effective amount of a molecule of claim 18 or a pharmaceutical composition comprising said molecule or a nucleic acid or set of nucleic acids encoding said molecule to said subject.

33. The method of claim 32, wherein the autoimmune disease, an inflammatory disease or disorder, or transplant rejection is selected from the group consisting of Myasthenia Gravis, Pemphigus vulgaris, systemic lupus erythematosus, Sjögren's syndrome, immune thrombocytopenia, chronic inflammatory demyelinating polyneuropathy, scleroderma, CREST syndrome, inflammatory myopathy, primary biliary cirrhosis, coeliac disease, rheumatoid arthritis, granulomatosis, antiphospholipid syndrome, Goodpasture syndrome, chronic autoimmune hepatitis, polymyositis, small intestinal bacterial overgrowth, Hashimoto's thyroiditis, Graves' disease, paraneoplastic cerebellar degeneration, limbic encephalitis, encephalomyelitis, subacute sensory neuronopathy, choreoathetosis, opsoclonus myoclonus syndrome, Stiff-Person syndrome, diabetes mellitus type 1, Isaac's syndrome, optic neuropathy, anti-N-Methyl-D-Aspartate Receptor Encephalitis, neuromyelitis optica, Bullous pemphigoid, membranous nephropathy, allogenic islet graft rejection, alopecia areata, ankylosing spondylitis, autoimmune Addison's disease, Alzheimer's disease, antineutrophil cytoplasmic autoantibodies (ANCA), autoimmune diseases of the adrenal gland, autoimmune hemolytic anemia, autoimmune myocarditis, autoimmune neutropenia, autoimmune oophoritis and orchitis, autoimmune urticaria, Behcet's disease, cardiomyopathy, Castleman's syndrome, celiac spruce-dermatitis, chronic fatigue immune disfunction syndrome, Churg-Strauss syndrome, cicatrical pemphigoid, CREST syndrome, cold agglutinin disease, Crohn's disease, dermatomyositis, discoid lupus, epidermolysis bullosa acquisita, essential mixed cryoglobulinemia, fibromyalgia-fibromyositis, glomerulonephritis, Guillain-Barre syndrome, graft-versus-host disease (GVHD), hemophilia A, idiopathic membranous neuropathy, idiopathic pulmonary fibrosis, idiopathic thrombocytopenia purpura (ITP), IgA neuropathy, IgM polyneuropathies, juvenile arthritis, Kawasaki's disease, lichen plantus, lichen sclerosus, Meniere's disease, mixed connective tissue disease, mucous membrane pemphigoid, multiple sclerosis, type 1 diabetes mellitus, Multifocal motor neuropathy (MMN), pemphigoid gestationis, pemphigus foliaceus, pernicious anemia, polyarteritis nodosa, polychrondritis, polyglandular syndromes, polymyalgia rheumatica, primary agammaglobulinemia, psoriasis, psoriatic arthritis, relapsing polychondritis, Reynauld's phenomenon, Reiter's syndrome, sarcoidosis, solid organ transplant rejection, Takayasu arteritis, toxic epidermal necrolysis (TEN), Stevens Johnson syndrome (SJS), temporal arteritis/giant cell arteritis, thrombotic thrombocytopenia purpura, ulcerative colitis, uveitis, dermatitis herpetiformis vasculitis, anti-neutrophil cytoplasmic antibody-associated vasculitides, vitiligo, asthma, autoimmune pancreatitis, IgA nephropathy and Wegner's granulomatosis.


SEQUENCE LISTING (29 sequences)

SEQ ID NO: 1 - protein, 365 aa, Homo sapiens
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 2 - protein, 274 aa, synthetic construct
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSS

SEQ ID NO: 3 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 4 - protein, 99 aa, synthetic construct [sequence omitted]

SEQ ID NO: 5 - protein, 641 aa, synthetic construct
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGGGGSGGGGSGGGGSAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSAAAHHHHHH

SEQ ID NO: 6 - protein, 641 aa, synthetic construct
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYAAAHHHHHH

SEQ ID NO: 7 - protein, 755 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYAAAHHHHHH

SEQ ID NO: 8 - protein, 580 aa, synthetic construct
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIEGRMDPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVTTLPPSRDELTKNQVSLTCLVKGFYPSDIAVEWESNGQPENNYKTFPPVLDSDGSFFLYSKLTVDKSRWQQGNVFSCSVMHEALKFHYTQKSLSLSPGK

SEQ ID NO: 9 - protein, 233 aa, synthetic construct
VEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSRDELTKNQVHLTCLVKGFYPSDIAVEWESNGQPENNYKTTPPVLDSDGSFALYSKLTVDKSRWQQGNVFSCSVMHEALKFHYTQKSLSLSPGK

SEQ ID NO: 10 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 11 - protein, 631 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDAAAHHHHHH

SEQ ID NO: 12 - protein, 755 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREALERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYAAAHHHHHH

SEQ ID NO: 13 - protein, 755 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSAESHLSLLYHLTAVSSPAPGTPAFWVSGWLGPQQYLSYNSLRGEAEPCGAAVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYAAAHHHHHH

SEQ ID NO: 14 - protein, 755 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSAESHLSLLYHLTAVSSPAPGTPAFWVSGWLGPQQYLSYNSLRGEAEPCGAWVAENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYAAAHHHHHH

SEQ ID NO: 15 - protein, 755 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSAESHLSLLYHLTAVSSPAPGTPAFWVSGWLGPQQYLSYNSLRGEAEPCGAWVWENQVSAYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYAAAHHHHHH

SEQ ID NO: 16 - protein, 755 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSAESHLSLLYHLTAVSSPAPGTPAFWVSGWLGPQQYLSYNSLRGEAEPCGAWVWENQVSWYAEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYAAAHHHHHH

SEQ ID NO: 17 - protein, 639 aa, synthetic construct
IQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSSETRPPLMYHLTAVSNPSTGLPSFWATGWLGPQQYLTYNSLRQEADPCGAWMWENQVSWYWEKETTDLKSKEQLFLEALKTLEKILNGTYTLQGLLGCELASDNSSVPTAVFALNGEEFMKFNPRIGNWTGEWPETEIVANLWMKQPDAARKESEFLLNSCPERLLGHLERGRRNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSSARSSGGGGSGGGGSGGGGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFAAAHHHHHH

SEQ ID NO: 18 - protein, 875 aa, synthetic construct
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGGGSGGGGSGGGGSAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYAAAHHHHHH

SEQ ID NO: 19 - protein, 36 aa, synthetic construct
  SITE 1: note = MISC_FEATURE - Xaa is d-alanine
  SITE 3: note = MISC_FEATURE - Xaa is a cyclohexylalanine
  SITE 13: note = MISC_FEATURE - Xaa is d-alanine
XKXVAAWTLKAAXNSNNLDSKVGGNYNYLYRLFRKS

SEQ ID NO: 20 - protein, 41 aa, synthetic construct
  SITE 1: note = MISC_FEATURE - Xaa is d-alanine
  SITE 3: note = MISC_FEATURE - Xaa is a cyclohexylalanine
  SITE 13: note = MISC_FEATURE - Xaa is d-alanine
XKXVAAWTLKAAXNYNYLYRLFRKSNLKPFERDISTEIYQA

SEQ ID NO: 21 - protein, 875 aa, synthetic construct
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGGGGSGGGGSGGGGSAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSAAAHHHHHH

SEQ ID NO: 22 - protein, 875 aa, synthetic construct
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDAAAHHHHHH

SEQ ID NO: 23 - protein, 875 aa, synthetic construct
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYAAAHHHHHH

SEQ ID NO: 24 - protein, 691 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 25 - protein, 457 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 26 - protein, 989 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYAAAHHHHHH

SEQ ID NO: 27 - protein, 989 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDAAAHHHHHH

SEQ ID NO: 28 - protein, 989 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGGGSGGGGSGGGGSAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYAAAHHHHHH

SEQ ID NO: 29 - protein, 982 aa, synthetic construct
IQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSSETRPPLMYHLTAVSNPSTGLPSFWATGWLGPQQYLTYNSLRQEADPCGAWMWENQVSWYWEKETTDLKSKEQLFLEALKTLEKILNGTYTLQGLLGCELASDNSSVPTAVFALNGEEFMKFNPRIGNWTGEWPETEIVANLWMKQPDAARKESEFLLNSCPERLLGHLERGRRNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSSARSSGGGGSGGGGSGGGGSGSEWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKDAVCSSSPSVVVSARTLNNRYTGPYTFALEDQPVKLPAVWSITTLNATSALLRAQEQIPPGVYHISLVLTDSQNNRCEMPRSLTLEVCQCDNRGICGTSYPTTSPGTRYGRPHSGRAAAHHHHHH